(19) United States
(12) Patent Application Publication
(10) Pub. No.: US 2023/0295654 A1
(43) Pub. Date: Sep. 21, 2023

(54) METHODS AND COMPOSITIONS FOR TREATMENT OF FRAGILE X SYNDROME

(71) Applicants: CHILDREN'S HOSPITAL MEDICAL CENTER, Cincinnati, OH (US); FORGE BIOLOGICS, INC., Grove City, OH (US)

(72) Inventors: Ernest PEDAPATI, Cincinatti, OH (US); Christina GROSS, Cincinatti, OH (US); Craig ERICKSON, Cincinatti, OH (US); David DISMUKE, Cary, NC (US); Erandi Kanchana DE SILVA, Columbus, OH (US)

(73) Assignees: CHILDREN'S HOSPITAL MEDICAL CENTER, Cincinnati, OH (US); FORGE BIOLOGICS, INC., Grove City, OH (US)

(21) Appl. No.: 18/005,757

(22) PCT Filed: Jul. 16, 2021
(86) PCT No.: PCT/US2021/041975

§ 371 Date: Jan. 17, 2023

Related U.S. Application Data
(60) Provisional application No. 63/053,461, filed on Jul. 17, 2020.

(51) Int. Cl. C12N 15/86; A61K 48/00; A61K 9/00; A61P 43/00

(52) U.S. Cl. CPC C12N 15/86; A61K 9/0019; A61K 48/005; A61P 43/00

(57) ABSTRACT

Methods for alleviating symptoms in a Fragile X Syndrome (FXS) patient using adeno-associated viral (AAV) 9 viral particles encoding a wild-type human fragile X mental retardation 1 (FMR1) protein (human FMRP). Also provided herein are methods to determine suitable doses of AAV9 viral particles for a FXS patient to alleviate at least one symptom associated with FXS, as well as methods for monitoring treatment efficacy.

### CROSS REFERENCE TO RELATED APPLICATIONS

This application claims the benefit of the filing date of U.S. Provisional Application No. 63/053,461, filed Jul. 17, 2020, the entire contents of which are incorporated by reference herein.

### BACKGROUND OF THE INVENTION

Fragile X Syndrome (FXS) is a monogenetic syndrome caused by an expansion of CGG repeats in the fragile X mental retardation protein (FMR1) gene which results in the loss of the gene product, the Fragile X mental retardation protein (FMRP), and the leading cause of inherited intellectual disability. Individuals with FXS have low IQs, are developmentally delayed, have impairments in verbal and nonverbal communication (often meeting ASD criteria), and suffer from neuronal hyperexcitability that becomes manifest in hypersensitivity to sound and light and in epileptic seizures.

Individuals with FXS need lifelong care and cannot live independent lives, reducing life quality for affected individuals and their caregivers. There is a need to develop new therapies for the treatment of FXS.

### SUMMARY OF THE INVENTION

The present disclosure is based, at least in part, on the development of AAV vectors that lead to successful in vivo expression of FMRP and the unexpected discoveries that a low level of FMRP expression mediated by AAV9 viral particles successfully improved primary behavioral symptoms of Fragile X Syndrome (FXS) in a mouse model. It was also discovered that electroencephalogram (EEG), behavioral assessments, cognitive neurorehabilitation assessments, or a combination thereof may be used as diagnostic and/or prognostic biomarkers, for example, for determining suitable doses (personalized doses) of FMR1-carrying AAV9 viral particles in alleviating symptoms in individual FXS patients and/or in assessing treatment efficacy.

Accordingly, one aspect of the present disclosure provides a method treating for treating FXS in a human patient by administering to a human patient having FXS an effective amount of a plurality of adeno-associated viral (AAV) 9 viral particles. The AAV9 viral particles can include a single-stranded AAV DNA vector, which may encompass a nucleotide sequence encoding a wild-type human fragile X mental retardation 1 (FMR1) protein (human FMRP) in operable linkage to a promoter. The AAV DNA vector may be a standard AAV vector. Alternatively, the AAV DNA vector may be a self-complementary AAV (scAAV) vector. The AAV DNA vector may express wild-type human FMRP in the brain of the human patient after infection of the AAV9 viral particles disclosed herein.

In some embodiments, the wild-type human FMRP can be human FMRP isoform 1. In other embodiments, the human FMRP may be a fragment of a wild-type human FMRP (e.g., isoform 1), which may comprise or consists of the N-terminal fragment of 1-297 amino acid residues.

In some embodiments, the promoter can be a hybrid of a chicken b-actin promoter and a CMV promoter. In other embodiments, the promoter may be a human phosphoglycerate kinase (hPGK) promoter.

In some embodiments, the AAV DNA vector may further comprise one or more regulatory elements regulating expression of human FMRP. For example, the one or more regulatory elements comprises a human β-globin intron sequence, one or more polyA signaling sequences, a woodchuck hepatitis virus post-transcriptional regulatory element (WPRE), or a combination thereof. In some examples, the one or more polyA signaling sequences comprise a human β-globin polyA signaling sequence, an SV40 polyA signaling sequence, or a combination thereof. In some examples, the AAV DNA vector does not contain a WPRE.

In specific examples, the AAV DNA vector is a standard AAV vector comprising a hybrid of a chicken β-actin promoter and a CMV promoter in operable linkage to the nucleotide sequence encoding human FMRP, a WPRE and an SV40 polyA signaling sequence downstream to the nucleotide sequence encoding the human FMR1.

In other specific examples, the AAV DNA vector is a standard AAV vector comprising a hybrid of a chicken β-actin promoter and a CMV promoter in operable linkage to the nucleotide sequence encoding human FMRP, and an SV40 polyA signaling sequence downstream to the nucleotide sequence encoding human FMRP. In some instances, the AAV DNA vector does not contain a WPRE.

In yet other specific examples, the AAV DNA vector is a standard AAV vector comprising is a human phosphoglycerate kinase (hPGK) promoter in operable linkage to the nucleotide sequence encoding human FMRP, a human β-globin intron sequence upstream to the nucleotide sequence encoding human FMRP, and SV40 polyA signaling and human β-globin polyA signaling sequences downstream to the nucleotide sequence encoding the human FMRP. In some instances, the AAV DNA vector does not contain a WPRE.

In some embodiments, the AAV DNA vector further includes one or more microRNA-target sites (MTSs) specific to one or more tissue-selective microRNAs to suppress expression of the wild-type FMRP in non-brain tissues. In some examples, one or more MTSs can be a MTS of miR-122, MTS of miR-208a, MTS of miR-208b-3p, MTS of miR-499a-3p, or a combination thereof.

In some embodiments, AAV9 viral particles disclosed herein can be administered to a human patient by intravenous injection, intracerebroventricular injection, intra-cisterna magna injection, intra-parenchymal injection, or a combination thereof. In some examples, AAV9 viral particles can be administered to a human patient via at least two administration routes. In some examples, the at least two administration routes can be intracerebroventricular injection and intravenous injection; intrathecal injection and intravenous injection; intra-cisterna magna injection and intravenous injection; or intra-parenchymal injection and intravenous injection.

In some embodiments, prior to administration of AAV9 viral particles disclosed herein, a human patient may be subject to electroencephalogram (EEG), behavioral and/or cognitive neurorehabilitation assessment, or a combination thereof for determining phenotypic severity of the disease. In some examples, the method can further include, prior to the administering step, subjecting the human patient to electroencephalogram (EEG), behavioral and/or cognitive neurorehabilitation assessment, or a combination thereof. In some examples, the method can further include, determining dosage of the AAV9 viral particles and/or delivery routes based on the EEG analysis, the behavioral and/or cognitive assessment, or the combination thereof.

In some embodiments, methods disclosed herein can be used on a human patient who has been undergoing or is undergoing a treatment comprising a GABA receptor agonist, a PI3K isoform-selective inhibitor, a MMP9 antagonist, or a combination thereof. In some examples, methods disclosed herein can further include administering to the human patient an effective amount of a GABA receptor agonist, a PI3K isoform-selective inhibitor, a MMP9 antagonist, or a combination thereof.

Another aspect of the present disclosure provides adeno-associated viral (AAV) vectors for expressing FMRP in a subject such as a human FXS patient and AAV particles comprising such a vector in single-strand form, as well as pharmaceutical compositions comprising such AAV viral particles.

In some embodiments, the AAV vector disclosed herein may include an AAV backbone, which comprises a 5′ inverted terminal repeats (ITR) and a 3′ ITR; a nucleotide sequence encoding a wild-type human fragile X mental retardation 1 (FMR1) protein (FMRP); a promoter in operable linkage to the nucleotide sequence encoding wild-type human FMRP; and, one or more microRNA-target sites (MTSs) specific to one or more tissue-selective microRNAs to suppress expression of the wild-type FMRP in non-brain tissues. In some examples, the AAV vectors disclosed herein can be a self-complementary AAV vector.

In some embodiments, the present disclosure features a standard adeno-associated viral (AAV) vector, comprising: (i) an AAV backbone, which comprises a 5′ inverted terminal repeats (ITR) and a 3′ ITR; (ii) a nucleotide sequence encoding a wild-type human fragile X mental retardation 1 protein (FMRP); (iii) a promoter in operable linkage to (ii); and (iv) one or more regulatory elements regulating expression of FMRP.

In some embodiments, the promoter is a hybrid of a chicken β-actin promoter and a CMV promoter. In other embodiments, the promoter is a human phosphoglycerate kinase (hPGK) promoter. Alternatively or in addition, the one or more regulatory elements comprises a human β-globin intron sequence, one or more polyA signaling sequences, a woodchuck hepatitis virus post-transcriptional regulatory element (WPRE), or a combination thereof. In some instances, the one or more polyA signaling sequences comprise a human β-globin polyA signaling sequence, an SV40 polyA signaling sequence, or a combination thereof. In some instances, the AAV DNA vector does not contain a WPRE.

In some examples, the AAV vector comprises a hybrid of a chicken β-actin promoter and a CMV promoter in operable linkage to the nucleotide sequence encoding human FMRP, a WPRE and an SV40 polyA signaling sequence downstream to the nucleotide sequence encoding the human FMRP.

In other examples, the AAV vector comprises a hybrid of a chicken β-actin promoter and a CMV promoter in operable linkage to the nucleotide sequence encoding human FMRP, and an SV40 polyA signaling sequence downstream to the nucleotide sequence encoding the human FMRP, and wherein the AAV DNA vector does not contain a WPRE.

In yet other examples, the AAV vector comprises a human phosphoglycerate kinase (hPGK) promoter in operable linkage to the nucleotide sequence encoding human FMRP, a human β-globin intron sequence upstream to the nucleotide sequence encoding human FMRP, and SV40 polyA signaling and human β-globin polyA signaling sequences downstream to the nucleotide sequence encoding the human FMRP, and wherein the AAV DNA vector does not contain a WPRE.

Also within the scope of the present disclosure are AAV9 particles as disclosed herein for use in treating FXS in a human patient and uses of the AAV9 particles for manufacturing a medicament for use in treating FXS.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A and 1B include diagrams depicting self-complementary AAV (scAAV) vectors capable of human FMRP production. FIG. 1A: Diagram depicts the scAAV plasmid structure for scAAV9-CB-FMR1 - a construct based on a scAAV backbone that contained the human FMR1 coding sequence under the control of the hybrid CMV enhancer/beta-actin promoter CB. FIG. 1B: Images depict western blot analysis of protein expression in primary cultured mouse cortical neurons transduced with increasing concentrations of scAAV viral genomes that contained either full length human FMRP, flag-tagged full length human FMRP, or GFP. Top panel depicts FMRP protein expression, middle panel depicts flag protein expression, and bottom panel depicts GFP protein expression.

FIGS. 2A-2C include diagrams depicting AAV (AAV) vectors capable of human FMRP production. FIG. 2A: Diagram depicts the AAV plasmid structure for AAV-CAG-FMR1 - a construct based on an AAV backbone that contained the human FMR1 coding sequence under the control of a CAG promoter. FIG. 2B: Images depict western blot analysis of protein expression in primary cultured mouse hippocampal neurons transduced with increasing concentrations of AAV viral genomes that contained either full length human FMRP or GFP. Top panel depicts FMRP protein expression, middle panel depicts GFP protein expression, and bottom panel depicts beta-actin protein expression (loading control). FIG. 2C: Graphs depict RT-PCR analysis of mRNA expression in primary cultured mouse hippocampal neurons transduced with increasing concentrations of AAV viral genomes that contained either full length human FMRP or GFP. Left panel depicts FMRP mRNA expression and right panel depicts GFP mRNA expression.

FIGS. 3A-3C include diagrams depicting virally expressed FMRP or GFP in cortical and hippocampal mouse neurons. FIG. 3A: Image depicts GFP expression in a mouse brain two weeks after intracerebroventricularly (ICV) injection of scAAV9-CB-GFP viral genomes. FIGS. 3B and 3C: Images depict FMRP expression in a mouse brain two weeks after ICV injection of AAV-CAG-FMRP viral genomes at 50 µm (FIG. 3B) and 100 µm (FIG. 3C). NeuN was used as an immunohistochemical marker of neuronal cells.

FIGS. 4A and 4B includes images of a western blot analysis for total protein expression of AAV-CAG-FMR1 and AAV-CAG-GFP in brain slices harvested from wild-type and Fmr1 knockout (KO) mice 10 weeks after mice were subjected to ICV injection of AAV-CAG-FMRP or AAV-CAG-GFP viral genomes. FIG. 4A: GFP. FIG. 4B: hFMRP.

FIG. 5 includes an image depicting a 10 week timeline for the study of behavior and functional assessments in Fmr1 KO and wild-type mice following AAV-CAG-FMRP or AAV-CAG-GFP administration.

FIGS. 6A-6C include diagrams depicting nesting assays performed in Fmr1 KO and wild-type mice following AAV-CAG-FMRP or AAV-CAG-GFP administration by ICV injection. FIG. 6A: Images show a shredded nestlet two hours after a fresh nestlet was provided to a wild-type, AAV-CAG-GFP-injected mouse (left panel) and a Fmr1 KO, AAV-CAG-GFP-injected mouse (right panel). FIG. 6B: Graph shows the percentage of nestlet shredded by Fmr1 KO and wild-type mice following AAV-CAG-FMRP or AAV-CAG-GFP administration where the nesting assay was performed once every four weeks after AAV injection. FIG. 6C: Graph shows the percentage of improvement in nesting behavior at two and four weeks after AAV injection in Fmr1 KO and wild-type mice injected with AAV-CAG-FMRP compared to Fmr1 KO and wild-type mice injected with AAV-CAG-GFP.

FIGS. 7A-7C include diagrams depicting marble burying assays performed in Fmr1 KO and wild-type mice following AAV-CAG-FMRP or AAV-CAG-GFP administration by ICV injection. FIG. 7A: Images show an example of marble burying behavior in wild-type, AAV-CAG-GFP-injected mouse and a Fmr1 KO, AAV-CAG-GF- injected mouse. FIG. 7B: Graph shows the latency to start burying marbles in Fmr1 KO and wild-type mice following AAV-CAG-FMRP or AAV-CAG-GFP administration. FIG. 7C: Graph shows the amount of marbles buried after 15 minutes by Fmr1 KO and wild-type mice following AAV-CAG-FMRP or AAV-CAG-GFP administration.

FIGS. 8A-8C include diagrams depicting Morris Water Maze assays performed on Fmr1 KO and wild-type mice six-eight weeks after AAV-CAG-FMRP or AAV-CAG-GFP administration by ICV injection. FIG. 8A: Images show a diagram of the Morris Water Maze assays that were performed as disclosed herein. FIG. 8B: Graph shows the number of entries into a quadrant that formally contained the hidden platform. FIG. 8C: Graph shows the latency to enter the former platform location in Fmr1 KO and wild-type mice following AAV-CAG-FMRP or AAV-CAG-GFP administration.

FIG. 9 includes a graph depicting the total amount of time AAV-CAG-FMR1- or AAV-CAG-GFP- injected Fmrl KO and wild type mice were in the open center during open field activity assays that measured hyperactivity and/or anxiety.

FIG. 10 includes a graph depicting the differences in preference of a novel object among AAV-CAG-FMR1- or AAV-CAG-GFP-injected Fmr1KO and wild type mice where the preference was calculated by the time spent interacting with the novel object divided by the amount of time exploring both the novel and familiar objects.

FIGS. 11A and 11B include diagrams depicting electrophysiological measurements of long-term potentiation in hippocampal slices prepared from the brains of AAV-CAG-FMR1-or AAV-CAG-GFP-injected Fmr1 KO and wild type mice 10 weeks after AAV administration by ICV injection. FIG. 11A: Graph shows long-term potentiation induced by theta-burst stimulation measured over 60 minutes. FIG. 11B: Graph shows long-term potentiation induced by theta-burst stimulation measured over 70 minutes.

FIGS. 12A and 12B include diagrams depicting protein synthesis rates in cortical slices prepared from brains harvested from AAV-CAG-FMR1-or AAV-CAG-GFP-injected Fmr1 KO and wild type mice 10 weeks after AAV administration by ICV injection. FIG. 12A: Image shows western blot analysis probing for puromycin-incorporation into nascent peptide chains following the treatment of cortical slices with vehicle (control) or puromycin. FIG. 12B: Graph depicts the beta-tubulin-normalized densitometry of puromycin abundance assessed by western blot analysis.

FIG. 13 includes a graph depicting increased gamma power in Fmrl KO compared to wild type (WT) mice where gamma power measured by continuous EEG was calculated for 5-minute periods over 6 days (n=3, RM 2-way ANOVA, *p<0.05).

FIGS. 14A-14D include diagrams depicting assessments of human data of gamma (y) power related abnormalities in Fragile X Syndrome (FXS). FIG. 14A: Excessive y power in FXS. Topographical plot of relative y power, including significant group differences (p < 0.05 corrected). FIG. 14B: Auditory cortex y power is highly correlated with behavior. Higher y is associated with lower performance on auditory attention task in FXS. FIG. 14C: y relationships with Theta and Alpha power highly discriminate between FXS (grey) and HC (black). FIG. 14D: EEG power analysis output from custom analysis software for murine EEG analysis.

FIG. 15 is a diagram depicting the plasmid map of the CAGWPRE vector.

FIG. 16 is a diagram depicting the plasmid map of the CAGdelWPRE vector.

FIG. 17 is a diagram depicting the plasmid map of the hPGK vector.

FIGS. 18A and 18B include photos showing expression of FMRP by vectors CAGWPRE (FIG. 18A) and CAGdelWPRE (FIG. 18B).

FIG. 19 is a photo showing expressing of FMRP by the CAGWPRE vector, the CAGdelWPRE vector, and the hPGK vector.

FIGS. 20A-20G include diagrams showing expression of FMRP and eGFP normalized to GAPDH in various tissues after administration of AAV particles carrying the AAV-CAG-FMR1 vector. The results were obtained by an RT-PCT assay. FIG. 20A: Cortex. FIG. 20B: Hippocampus. FIG. 20C: Midbrain. FIG. 20D: Cerebellum. FIG. 20E: Heart. FIG. 20F: Liver. FIG. 20G: Kidney.

### DETAILED DESCRIPTION OF THE INVENTION

Fragile X Syndrome (FXS) also known as Martin-Bell syndrome or Escalante’s syndrome, is a genetic disorder resulting from an expansion of the CGG trinucleotide repeat in the FMR1 gene on the X chromosome. The expanded CGG trinucleotide repeat responsible for FXS is located in the 5′ untranslated region (UTR) of the FMR1 gene which encodes the fragile X mental retardation protein (FMRP), which is required for normal neural development. A trinucleotide repeat (CGG) in the 5′ UTR is normally found at 6-53 copies; however, individuals affected with FXS generally have 55-230 repeats of the CGG codon, which results in methylation of the FMR1 promoter, silencing of the gene, and a failure to produce FMRP.

FMRP associates with hundreds of mRNAs regulating their translation and stability and can also directly affect neuronal excitability by binding ion channels at synapses. Consequently, loss of FMRP leads to a plethora of molecular, cellular and structural defects that are difficult, if not impossible, to correct with single-drug strategies in humans. The resulting defects occurring in the absence of FMRP can result in cognitive disability, communication deficits, social skill deficits, sensory sensitivity, inattention, adaptive behavior deficits, anxiety, autonomic system dysregulation, and seizure.

The present disclosure aims at developing treatment of FXS with AAV9 viral particles containing a nucleic acid for expressing a functional (e.g., wild-type) human fragile X mental retardation 1 (FMR1) protein (FMRP) to improve behavioral and functional symptoms associated with FXS.

The present disclosure reports development of various AAV vectors, which led to successful expression of FMRP in a mouse model. Surprisingly, a low level of FMRP expression via delivery of AAV9 viral particles encoding FMRP into the CNS of an animal model of FXS successfully alleviated symptoms associated with FXS as observed in the FXS mouse model. Further, the present disclosure reports that electroencephalogram (EEG), behavioral, cognitive neurorehabilitation assessment, or a combination thereof can be used as diagnostic and/or prognostic biomarkers, for example, for assessing proper dosage of AAV9 viral particles encoding FMRP for an individual FXS patient. In addition, such biomarkers can be used for assessing treatment efficacy.

The present disclosure established evidence of heightened cortical excitability in a well-powered sample of FXS with age- and gender-matched controls. By source localizing dense-array EEG data, three major findings of interest were identified: (i) focal increases gamma oscillations within functional resting state networks and cortical regions, (ii) marked alterations in low-frequency power and coupling relationships, and (iii) independent of case-control contrasts, source-estimated gamma power from the default mode network is highly predictive of disease-specific intellectual disability. These findings support an effective method of parsing heterogeneity within FXS as a “disease of networks” and cortical hyperexcitability and provides a feasible method of measuring these changes and clinical relevance to intellectual disability in FXS, which may be used as biomarkers for identify suitable patients for treatment and/or monitoring treatment efficacy.

Accordingly, provided herein are AAV9 viral vectors and particles for expressing FMRP and uses thereof in alleviating FXS symptoms in FXS patients. Also provided herein are methods for making the disclosed AAV9 viral particles and determining suitable doses (personalized doses) of AAV9 viral particles for an individual FXS patient using one or more of the behavior features disclosed herein as a biomarker.

### I. AAV Viral Particles for Expressing a FMR1 Protein

In one aspect, the present disclosure provides AAV viral particles (e.g., AAV9 viral particles) for use as a vehicle for delivering FMRP to a subject in need of the treatment of FXS.

Adeno-associated virus (AAV), a member of the Parvovirus family, is a small, non-enveloped virus. AAV particles here may include an AAV capsid composed of capsid protein subunits, VP1, VP2 and VP3, which enclose a single-stranded DNA genome. The properties of non-pathogenicity, broad host range of infectivity, including non-dividing cells, and lack of integration make AAV an attractive gene delivery vehicle.

As used herein, an AAV viral particle contains an AAV DNA vector encapsulated by viral capsid proteins. An AAV viral particle is capable of infecting certain tissues and cells depending upon its serotype. See descriptions below. The AAV DNA vector (or AAV vector) refers to the DNA molecule carried in a viral particle that includes a nucleotide sequence encoding a wild-type human fragile X mental retardation 1 (FMR1) protein (FMRP), and optionally regulatory elements for controlling expression of FMRP. The regulatory elements can be selected for modulating the expression level of FMRP and/or for improving safety. For example, the FMR1 coding sequence can be in operable linkage to a suitable promoter that drives expression of FMRP. In some instances, the AAV DNA vector may comprise one or more regulatory elements that regulate expression of FMRP, for example, one or more miRNA binding sites, enhancers, transcriptional factor binding sites, polyA signaling elements, or a combination thereof.

### (A) FMRP Protein

The AAV viral particles disclosed herein such as AAV9 viral particles carry an AAV vector for expressing a functional FMRP. FMR1 is an mRNA-binding protein that is highly expressed in brain where it transport certain mRNAs from the nucleus to neuronal synapses. In the absence of FMRP, synapses do not form appropriately, leading to decreased cognitive capacity and developmental impairment associated with FXS.

In some embodiments, the FMRP disclosed herein may be a naturally-occurring FMRP. A naturally-occurring FMRP or subunit may be from a suitable species, e.g., from a mammal such as mouse, rat, rabbit, pig, a non-human primate, or human. In some examples, the FMRP is a wild-type human protein. Naturally-occurring FMRP from various species are well known in the art and their sequences can be retrieved from a public gene database such as GenBank.

The structure of a naturally-occurring human FMRP contains multiple conserved functional domains. For example, the functional domains of FMRP consist of two tudor domains, a nuclear localization signal (NLS), three K homology domains (KH0, KH1, KH2), a nuclear export signal (NES) and an arginine-glycine-glycine domain (RGG) from N- to C-terminus. The tudor, KH and RGG domains are mainly involved in RNA binding, though they also have protein interaction partners.

The FMR1 gene is a highly conserved gene that consists of 17 exons spanning approximately 38 kb of genomic DNA. The FMR1 gene undergoes extensive alternative splicing yielding different FMR1 transcriptional isoforms, resulting in several FMRP isoforms. FMR1 transcriptional isoforms can be categorized into groups by their exon structures as shown in Table 1 below.

TABLE 1

Splice pattern grouping of FMR1 transcriptional isoforms

| | |
|---|---|
| Group | Exons |
| A | 9,10,11,12,13,14,15,16,17 |
| B | 9,10,11,12,13,15,16,17 |
| C | 9,10,11,13,14,15,16,17 |
| D | 9,10,11,13,15,16,17 |
| E | 9,10,15,16,17 |
| F | Different combination of exons |

The human FMR1 gene can produce a total of 11 FMRP isoforms as a result of alternative splicing. These FMRP isoforms share a highly conserved N-terminal fragment of ~400 residues and variable C-terminal sequences with varying mRNA-binding affinities. Any of the splice isoforms of FMR1 can be used in the present disclosure. In some examples, the human FRMP used herein is FRMP isoform 1. The amino acid sequence of human FMRP isoform 1 is provided below (SEQ ID NO: 1)

MEELVVEVRGSNGAFYKAFVKDVHEDSITVAFENNWQPDRQIPFHDVRFP
PPVGYNKDINESDEVEVYSRANEKEPCCWWLAKVRMIKGEFYVIEYAACD
ATYNEIVTIERLRSVNPNKPATKDTFHKIKLDVPEDLRQMCAKEAAHKDF
KKAVGAFSVTYDPENYQLVILSINEVTSKRAHMLIDMHFRSLRTKLSLIM
RNEEASKQLESSRQLASRFHEQFIVREDLMGLAIGTHGANIQQARKVPGV
TAIDLDEDTCTFHIYGEDQDAVKKARSFLEFAEDVIQVPRNLVGKVIGKN
GKLIQEIVDKSGVVRVRIEAENEKNVPQEEEIMPPNSLPSNNSRVGPNAP
EEKKHLDIKENSTHFSQPNSTKVQRVLVASSVVAGESQKPELKAWQGMVP
FVFVGTKDSIANATVLLDYHLNYLKEVDQLRLERLQIDEQLRQIGASSRP
PPNRTDKEKSYVTDDGQGMGRGSRPYRNRGHGRRGPGYTSGTNSEASNAS
ETESDHRDELSDWSLAPTEEERESFLRRGDGRRRGGGGRGQGGRGRGGGF
KGNDDHSRTDNRPRNPREAKGRTTDGSLQIRVDCNNERSVHTKTLQNTSS
EGSRLRTGKDRNQKKEKPDSVDGQQPLVNGVP

Exemplary coding sequence for the FMRP can be found under GenBank accession no. NM_002024.

In some embodiments, the FMRP to be produced by the AAV particles disclosed herein may a functional fragment of a naturally-occurring human FMRP. Such a functional fragment may include one or more of the FMRP functional domains disclosed herein. In some instances, the functional fragment comprises the ~400 amino acid-long N-terminal conserved domain of a wild-type FMRP. In some examples, the fragment of an FMRP may comprise (e.g., consisting of) the N-terminal 1-297 amino acid residues. Alternatively or in addition, the functional fragment may comprise at least one tudor domain, a least one NLS, at least one KH, at least one NES, at least one RGG, or a combination thereof. In some examples, the functional fragment may have a truncation at the N-terminus as relative to the wild-type counterpart. In other examples, the functional fragment may have a truncation at the C-terminus as relative to the wild-type counterpart. In some instances, the functional fragment may have truncations at both the N-terminus and the C-terminus relative to the wild-type counterpart.

In some embodiments, the FMRP to be produced by the AAV particles disclosed herein may be a functional variant of a naturally-occurring FMR1 (e.g., a functional variant of human FMR1 isoform 1). Such a functional variant shares a high sequence homology (e.g., at least 85%, at least 90%, at least 95%, or above) with the naturally-occurring FMR1 counterpart (e.g., SEQ ID NO:1) and has substantially similar bioactivity as the naturally-occurring FMR1 counterpart (e.g., at least 80% of a bioactivity as compared with the wild-type counterpart).

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Any functional variant disclosed herein may comprise one or more of the functional domains of a wild-type FMRP such as those described herein, e.g., the N-terminus conserved domain, the tudor domains, the KH domains, and/or the RGG domains and comprise one or more variations in one or more non-functional domains. Alternatively, the functional variant may contain conservative amino acid residue substitutions relative to the wild-type counterpart, for example, in one or more functional domains, and/or in one or more non-functional domains.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some examples, the FMRP encoded by the transgene in any of the AAV vectors disclosed herein may comprise a signal peptide at the N-terminus, which will secretion of the FMRP from the host cells. Examples of such signal peptides include the signal peptide from an albumin, a β-glucuronidase, an alkaline protease or a fibronectin.

In other examples, the FMRP disclosed herein may be a fusion protein comprising vectors disclosed herein may include a nucleic acid transgene is fused with a protein motif that improves secretion of the FMRP, for example, a protein transduction domain (PTD), such as the PTD from Tat or VP22.

### (B) AAV Vectors

An AAV vector comprises necessary genetic elements derived from a wild type genome of the virus (viral backbone elements) such that the vector can be packaged into viral particles and express the transgene(s) carried therein in host cells. Further, the AAV vectors disclosed herein comprises a coding sequence for a FMRP disclosed herein, and a suitable promoter in operable linkage to the coding sequence. In some examples, the AAV vector disclosed herein can further comprise one or more regulatory sequences regulating expression and/or secretion of the encoded FMRP. Examples include, but are not limited to, enhancers, intron sequences, polyadenylation signal sites, internal ribosome entry sites (IRES), microRNA-target sites, posttranscriptional regulatory elements (PREs; e.g., woodchuck hepatitis virus posttranscriptional regulatory element (WPRE)), or a combination thereof. Elements that may raise safety concerns may be excluded.

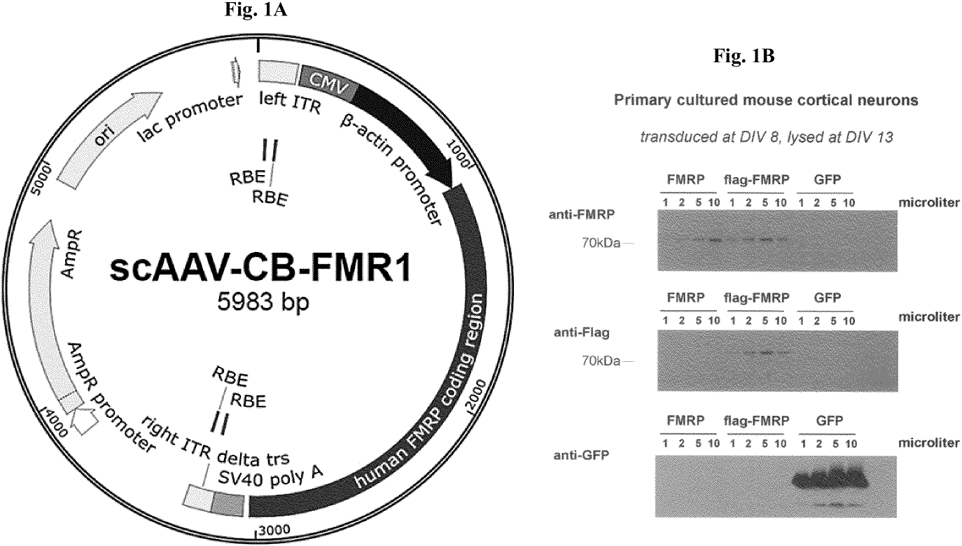

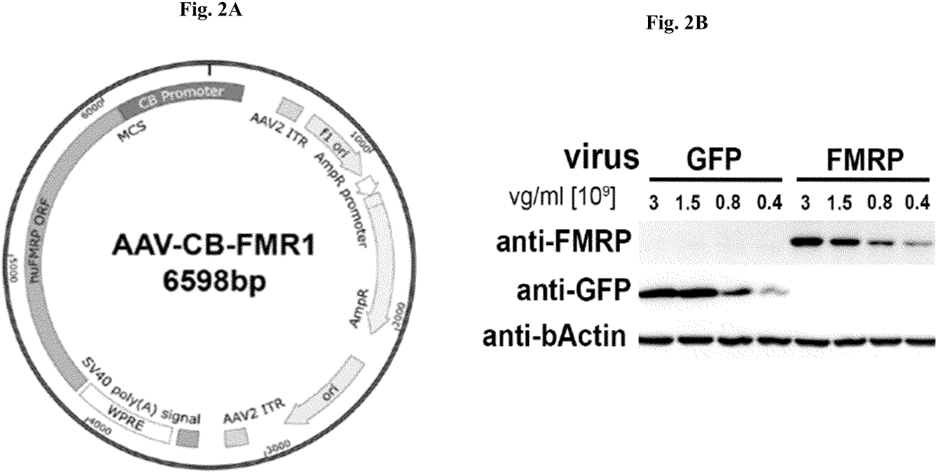

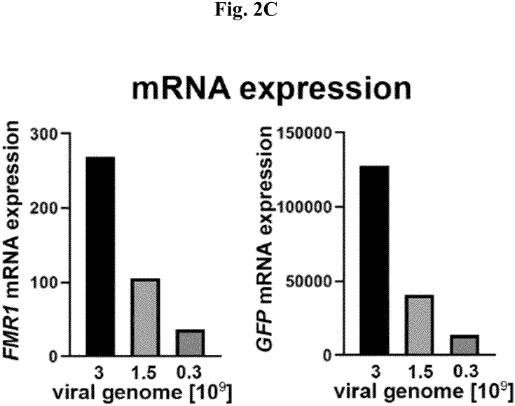

In some examples, the AAV vector may be a regular (standard) AAV vector comprising a single stranded nucleic acid. See, e.g., FIG. 2A and FIGS. 15-17 as examples. In other examples, the AAV vector disclosed herein may be a self-complementary AAV vector capable of comprising double stranded portions therein. See, e.g., FIG. 1A as an example.

### Viral-Backbone Elements

The AAV vectors disclosed herein comprises one or more AAV-genome derived backbone elements, which refer to the minimum AAV genome elements required for the bioactivity of the AAV vectors. For example, the AAV-genome derived backbone elements may comprise the packaging site for the AAV vector to be assembled into an AAV viral particle, elements needed for vector replication and/or expression of the transgene comprised therein in host cells. In some examples, commercially available AAV vectors (e.g., from Addgene) may be used here. For example, an AAV vector provided by Addgene (e.g., Addgene plasmid #28014) may be used and the GFP gene contained therein may be replaced with the coding sequence for FMR1.

Virus-derived elements for use in an AAV vector are well known in the art. Typically, an AAV vector would comprise one or both inverted terminal repeat (ITR) sequences derived from a wild type AAV genome. In some examples, the ITR sequences in an AAV vector disclosed herein may be wild-type. In other examples, the ITR sequences used in an AAV vector may be a modified version of a wild-type ITR (e.g., a truncated version). ITRs for use in constructing AAV vectors, including wild-type or modified versions, are also well known in the art. See, e.g., Daya et al., Clinical Microbiology Reviews, 21(4):583-593 (2008), the relevant disclosures of which are incorporated by reference for the subject matter and purpose referenced herein. In some examples, AAV2 ITRs may be used.

In some examples, the viral backbone elements disclosed herein may include at least one inverted terminal repeat (ITR) sequence, for example, two ITR sequences. In some examples, one ITR sequence is 5′ of the coding sequence for FMRP. In other examples, one ITR sequence is 3′ of the coding sequence. In some examples, a polynucleotide sequence coding for FMRP is flanked by two ITR sequence in the AAV vector disclosed herein. In some examples, a polynucleotide sequence coding for FMRP can be flanked by two stuffer sequences in an AAV vector disclosed herein.

### Self-Complementary AAV Viral Vectors

In some embodiments, the AAV vector disclosed herein is a self-complementary AAV (scAAV) vector. Self-complementary AAV (scAAV) vectors contains complementary sequences that are capable of spontaneously annealing (folding back on itself to form a doublestranded genome) when entering into infected cells, thus circumventing the need for converting a single-stranded DNA vector using the cell’s DNA replication machinery. Self-complementing AAV vectors are known in the art. See, e.g., U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, the relevant disclosures of each of which are incorporated herein by reference for the purpose and subject matter referenced herein. An AAV comprising a self-complementing genome can quickly form a double stranded DNA molecule by virtue of its partially complementing sequences (e.g., complementing coding and non-coding strands of a transgene), thereby rapidly producing the encoded protein.

In some embodiments, the scAAV viral vector disclosed herein may comprise a first heterologous polynucleotide sequence (e.g., an FMR1 coding strand) and a second heterologous polynucleotide sequence (e.g., an FMR1 noncoding or antisense strand), which form intrastrand base pairs. In some examples, the first heterologous polynucleotide sequence and the second heterologous polynucleotide sequence are linked by a sequence that facilitates intrastrand base pairing; e.g., to form a hairpin DNA structure.

In some examples, the dimeric structure of a scAAV vector upon entering a cell can be stabilized by a mutation or a deletion of one of the two terminal resolution sites (trs). As trs are Rep-binding sites contained within each ITR, a mutation or a deletion of such trs may prevent cleavage of a dimeric structure of a scAAV vector by AAV Rep proteins to form monomers.

In some examples, a scAAV viral vector disclosed herein may include a truncated 5′ inverted terminal repeats (ITR), a truncated 3′ ITR, or both. In some examples, the scAAV vector disclosed herein may comprise a truncated 3′ ITR, in which the D region or a portion thereof (e.g., the terminal resolution sequence therein) may be deleted. Such a truncated 3′ ITR may be located between the first heterologous polynucleotide sequence and a second heterologous polynucleotide sequence noted above.

### Promoters

In some embodiments, the AAV vectors disclosed herein can include one or more suitable promoters in operable linkage to the FMR1 coding sequence for controlling expression of the encoded FMRP in suitable host cells such as human brain cells. Such a promoter may be ubiquitous, tissue-specific, strong, weak, regulated, chimeric, etc., to allow efficient and suitable production of the protein in the host cells. The promoter may be homologous to the encoded protein, or heterologous, including cellular, viral, fungal, plant or synthetic promoters. In some examples, the promoter used in any of the AAV vectors disclosed herein is functional in human cells, for example, functional in brain cells. Non-limiting examples of ubiquitous promoters include viral promoters, particularly the CMV promoter, the RSV promoter, the SV40 promoter, etc. and cellular promoters such as the PGK (phosphoglycerate kinase) promoter (e.g., human PGK promoter).

In some examples, the AAV vector disclosed herein may comprise a brains specific promoter for controlling expression of the FMR1 transgene therein. Such a brain specific promoter may drive expression of the transgene in brain tissues at least 2-fold, 5-fold, 10-fold, 20-fold, 50-fold or 100-fold higher than in a non-brain cell. In other examples, the promoter can be an endothelial cell-specific promoter such as the VE-cadherin promoter. In yet other examples, the promoter may be a steroid promoter or a metallothionein promoter. Preferably, this promoter is a human promoter.

In some examples, the AAV vector disclosed herein may comprise the cytomegalovirus (CMV) promoter in operable linkage to the coding sequence of the FMRP. In some instances, the CMV promoter is a wild-type CMV promoter. In other examples, the AAV vector may comprise the chicken beta-actin gene promoter. In specific examples, the AAV vector may comprise a hybrid CMV/chicken beta-actin promoter. For example, the AAV vector may comprise the synthetic CAG promoter, which contains the CMV early enhancer element, the promoter, the firs exon and first intron of the chicken beta-actin gene, and the splice acceptor of the rabbit beta-globin gene. A nucleotide sequence of the CAG promoter is provided below: Modified CAG sequence (SEQ ID NO: 2):

attgacgtcaataatgacgtatgttcccatagtaacgccaatagggactt
tccattgacgtcaatgggtggagtatttacggtaaactgcccacttggca
gtacatcaagtgtatcatatgccaagtacgccccctattgacgtcaatga
cggtaaatggcccgcctggcattatgcccagtacatgaccttatgggact
ttcctacttggcagtacatctacgtattagtcatcgctattaccatggtc
gaggtgagccccacgttctgcttcactctccccatctcccccccctcccc
acccccaattttgtatttatttattttttaattattttgtgcagcgatgg
gggcgggggggggggggggggggcggggcgaggcggagaggtgcggcggc
agccaatcagagcggcgcgctccgaaagtttccttttatggcgaggcggc
ggcggcggcggccctataaaaagcgaagcgcgcggcgggcgggagtcgct
gcgcgctgccttcgccccgtgccccgctccgccgccgcctcgcgccgccc
gccccggctctgactgaccgcgttactcccacaggtgagcgggcgggacg
gcccttctcctccgggctgtaattagcgcttggtttaatgacggcttgtt
tcttttctgtggctgcgtgaaagccttgaggggctccgggagggcccttt
gtgcggggggagcggctcggggctgtccgcggggggacggctgccttcgg
gggggacggggcagggcggggttcggcttctggcgtgtgaccggcggctc
tagagcctctgctaaccatgttcatgccttcttctttttcctacagctcc
tgggcaacgtgctggttattgtgctgtctcatcattttggcaaagaatt

In other examples, the AAV vector disclosed herein may comprise a PGK promoter, such as a human PGK promoter. One example is provided below: hPGK Promoter Sequence (SEQ ID NO: 3)

GGGGTTGGGGTTGCGCCTTTTCCAAGGCAGCCCTGGGTTTGCGCAGGGAC
GCGGCTGCTCTGGGCGTGGTTCCGGGAAACGCAGCGGCGCCGACCCTGGG
TCTCGCACATTCTTCACGTCCGTTCGCAGCGTCACCCGGATCTTCGCCGC
TACCCTTGTGGGCCCCCCGGCGACGCTTCCTGCTCCGCCCCTAAGTCGGG
AAGGTTCCTTGCGGTTCGCGGCGTGCCGGACGTGACAAACGGAAGCCGCA
CGTCTCACTAGTACCCTCGCAGACGGACAGCGCCAGGGAGCAATGGCAGC
GCGCCGACCGCGATGGGCTGTGGCCAATAGCGGCTGCTCAGCAGGGCGCG
CCGAGAGCAGCGGCCGGGAAGGGGCGGTGCGGGAGGCGGGGTGTGGGGCG
GTAGTGTGGGCCCTGTTCCTGCCCGCGCGGTGTTCCGCATTCTGCAAGCC
TCCGGAGCGCACGTCGGCAGTCGGCTCCCTCGTTGACCGAATCACCGACC
TCTCTCCCCAG

### MicroRNA-Target Sites

In some embodiments, AAV vectors disclosed herein may include at least one miRNA target site (MTS). As used herein, “miRNA target site” or “miRNA target sequence” refers to a nucleic acid sequence, to which a miRNA specifically binds. Translation of an mRNA transcribed from an AAV vector comprising one or more miRNA binding site would usually be blocked (silenced) when the corresponding miRNA binds the miRNA target site, which may lead to destabilization of the mRNA. A miRNA target site may comprise a nucleotide sequence complementary (completely or partially) to a corresponding miRNA such that the miRNA can form base pairs at the miRNA target site. In some examples, the one or more miRNA target sites are located 3′ downstream of the FMR1 coding sequence. In that case, the resultant mRNA would comprise the miRNA target sequences at the 3′ untranslated region (3′ UTR).

In some examples, an AAV vector disclosed herein may include one or more microRNA-target sites (MTSs) specific to one or more tissue-selective microRNAs to suppress expression of FMRP in non-brain tissues. In some examples, at least one MTS can suppress FMRP in non-brain tissue by at least 2-fold, 5-fold, 10-fold, 20-fold, 50-fold or 100-fold compared to a vector lacking the MTS. In some examples, the AAV vector may comprise at least one MTS that can be bound by miRNAs specific to non-brain organs such as liver, lung, pancreas, kidney, heart, etc. so at to block expression of FMR1 in such organs.

In some examples, an AAV vector disclosed herein may comprise a MTS specific to miR122. miR122 is enriched in the liver, and also expressed in thryroid, spleen, and lung. Low levels of expression of miR122 were observed in pancreas, kidney, and artery. In other examples, an AAV vector disclosed herein may comprise a MTS specific to miR-208a or miR-208b-3p, which are enriched in myocardium, muscle, also expressed in thyroid at lower level. In yet other examples, an AAV vector disclosed herein may comprise a MTS specific to miR-499a-3p, which is enriched in myocardium, muscle, also in thyroid, prostate, and bone. Additional suitable MTSs for use in the AAV vectors disclosed herein are known in the art, for example, provided in Luwig et al., Nucleic Acid Res. 44(8):3865-3877 (2016), the relevant disclosures of which are incorporated by reference for the subject matter and purpose referenced herein. In specific examples, an AAV vector disclosed herein may comprise a combination of tissue-specific miRNA target sites such as those disclosed herein.

### Other Regulatory Elements for Gene Expression

In some embodiments, the AAV vectors disclosed herein may further include one or more regulatory elements, which can be operably linked to the transgene (coding for FMRP) for regulating expression of FMRP in brain cells. Exemplary regulatory elements include, but are not limited to, transcription initiation sites and/or termination sites, enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including native, constitutive, inducible and/or tissue-specific, are known in the art and may be utilized in the present disclosure.

For example, the AAV vector may comprise a polyadenylation sequence, such as the SV40 polyadenylation sequences or polyadenylation sequences of bovine growth hormone. In some instances, the AAV vector may comprise one or more intron sequences, one or more polyA signaling sequences, and/or one or more posttranscriptional regulatory elements. Elements that may raise safety concerns, for example, the woodchuck hepatitis virus posttranscriptional regulatory elements (WPRE), may be excluded, in some instances.

### Exemplary Examples of AAV Vectors

In some examples, the AAV vector disclosed herein may comprise (a) an AAV viral backbone, which may contain a 5′ inverted terminal repeat (ITR) and a 3′ ITR; (a) a nucleotide sequence encoding a functional human fragile X mental retardation 1 (FMR1) (e.g., human FMR1 isoform 1) protein (FMRP); (c) a promoter in operable linkage to the FMRP-coding sequence, and (d) one or more microRNA-target sites (MTSs). In some instances, the promoter may be a hybrid of a chicken β-actin promoter and a CMV promoter (e.g., the CAG promoter). Alternatively or in addition, the one or more tissue-selective miRNA target sites may be specific to one or more miRNAs that present in non-brain tissues but not in brain cells (or only at a very low level such that expression of FMRP would not be affected significantly). Exemplary MTSs include those specific to miR-122, miR-208a, miR-208b-3p, miR-499a-3p, or a combination thereof. Such an AAV vector may further comprise one or more of the regulatory elements disclosed herein.

In other examples, the AAV vector provided herein is a self-complementary AAV (scAAV) vector, comprising (a) a 5′ inverted terminal repeat (ITR) and a 3′ ITR, either one of which or both of which are truncated; (b) a nucleotide sequence encoding a wild-type human FMR1 isoform 1 protein; (c) a promoter in operable linkage to the FMRP-encoding nucleotide sequence. In some instances, the promoter is a hybrid of a chicken β-actin promoter and a CMV promoter (e.g., the CAG promoter). In some instances, the scAAV may further comprise one or more microRNA-target sites (MTSs), which may be specific to one or more miRNAs that present in non-brain tissues but not in brain cells (or only at a very low level such that expression of the FMRP would not be affected significantly). Exemplary MTSs include those specific to miR-122, miR-208a, miR-208b-3p, miR-499a-3p, or a combination thereof. Such a scAAV vector may further comprise one or more of the regulatory elements disclosed herein.

scAAV vectors are generally known as having a limited insertion capacity. As such, this type of AAV vectors is commonly viewed as not suitable for large transgenes. Here, a scAAV vector was used to successfully clone the coding sequence of the full-length human FMR1 isoform 1 and express the encoded FMR1 isoform 1 protein (FMRP isoform 1). This data suggests that scAAV vectors would be suitable for use in delivering the large full-length FMR1 isoform 1 protein (FMRP isoform 1) for gene therapy purposes.

In some examples, the AAV vector provided herein may be a standard (regular) AAV vector comprising: an AAV backbone, which comprises a 5′ inverted terminal repeats (ITR) and a 3′ ITR; (ii) a nucleotide sequence encoding a wild-type human fragile X mental retardation 1 (FMR1) protein; (iii) a promoter in operable linkage to (ii); and (iv) one or more regulatory elements regulating expression of FMRP. The promoter may be a CAG promoter as disclosed herein. Alternatively, the promoter may be a PGK promoter as also disclosed herein. In some instances, the AAV vector comprises one or more regulatory elements, which may be one or more intron sequences (e.g., a human β-globin intron sequence), one or more polyA signaling sequences (e.g., SV40 polyA signaling sequence, human β-globin polyA signaling sequence, or a combination thereof), one or more posttranscriptional regulatory elements (e.g., WRPE), or a combination thereof. In other instances, the AAV vector provided herein may not contain WRPE or the like to improve safety.

Specific examples of the AAV vectors disclosed herein are provided in Example 1 below.

### (C) Serotype of AAV Viral Particles

The AAV viral particles may be of a suitable serotype that is capable of infecting brain cells. There are eleven serotypes of AAV virus identified to date. These serotypes differ in the types of cells they infect. In some embodiments, the AAV viral particles disclosed herein can be AAV1, AAV2, AAV4, AAV5, AAV8, or AAV9, all of which are capable of infecting brain cells. In some examples, the AAV viral particle is AAV9.

In some examples, the AAV viral particle may be a hybrid AAV comprising genomic elements from one serotype and capsid from at least another serotype. For example, the AAV vector may comprise genomic elements from AAV2 (e.g., AAV2 ITRs, wild-type or modified versions) and capsid from one of the serotypes capable of infecting brain cells (e.g., AAV9).

In some embodiments, an AAV viral particle disclosed herein may include a modified capsid, for example, by a non-viral protein or a peptide or by structural modification, to alter the tropism of the AAV viral particle such that it would be capable of infecting brain cells. For example, the capsid may include a ligand of a brain cell receptor (e.g., a brain cell specific receptor) such that the AAV viral particle comprising such could target and infect brain cells.

### (D) Methods of Making AAV Particles

The AAV DNA vector constructs disclosed herein may be prepared using known techniques, for example, recombinant technology. See, e.g., Current Protocols in Molecular Biology, Ausubel., F. et al., eds, Wiley and Sons, New York 1995). In some instances, size of the transgene and regulatory elements can be designed so as to meet the packaging capacity of the AAV particle. If necessary, a “stuffer” DNA sequence can be added to the construct to maintain standard AAV genome size for comparative purposes. Such a fragment may be derived from such non-viral sources known and available to those skilled in the art.

An AAV DNA vector may be packaged into virus particles, which can be used to deliver the transgene to host cells for expression. For example, an AAV vector as disclosed herein can be transfected into a producer cell lines (packaging cells) capable of producing viral proteins such as capsid proteins necessary for AAV virion package.

A packaging cell line may be generated by establishing a cell line that are stably transfected with all of the necessary components for AAV particle production, for example, AAV rep and cap genes, and optionally a selectable marker, such as a neomycin resistance gene. See, e.g., Samulski et al., 1982, Proc. Natl. Acad. S6. USA, 79:2077-2081. In some instances, the packaging cell line can be infected with a helper virus, such as adenovirus, in producing AAV viral particles. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells. General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658.776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Patent. No. 5,786,211; U.S. Pat. No. 5,871,982; and U.S. Pat. No. 6,258,595.

### II. Pharmaceutical Compositions

Any of the AAV viral particles (e.g., AAV9 viral particles) disclosed herein may be formulated to form a pharmaceutical composition, which may further comprise a pharmaceutically acceptable carrier, diluent or excipient. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some embodiments, the pharmaceutical compositions or formulations are for parenteral administration, such as intravenous, intracerebroventricular injection, intra-cisterna magna injection, intra-parenchymal injection, or a combination thereof. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating AAV particles in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

### III. Treatment of FXS With AAV Particles Producing: FMRP

Any of the AAV particles carrying a viral vector coding for FMRP as disclosed herein can be used to deliver the FMRP-encoding transgene to brain cells for FMRP expression to alleviate one or more symptoms associated FXS. Thus, in some aspects, the present disclosure provides methods for alleviating one or more symptoms and/or for treating FXS in a subject in need of the treatment a plurality of AAV particles such as AAV9 particles disclosed herein, as well as a pharmaceutical composition comprising such. To perform the method disclosed herein, an effective amount of the AAV particles or a pharmaceutical composition comprising such may be administered to a subject who needs treatment via a suitable route (e.g., intravenous, intracerebroventricular injection, intra-cisterna magna injection, or intra-parenchymal injection) at a suitable amount as disclosed herein.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

A subject to be treated by any of the methods disclosed herein may be a human patient having FXS, who can be identified by routine medical examination, e.g., laboratory tests, organ functional tests, behavioral tests, CT scans, electroencephalogram, and/or magnetic resonance imaging (MRI). FXS patients typically have one or more genetic mutations in the FMR1 gene, which usually makes a protein called fragile X mental retardation protein (FMRP), also referred to as FMRP. Nearly all cases of fragile X syndrome are caused by a mutation, in which a DNA segment, known as the CGG triplet repeat, is expanded within the FMR1 gene. Normally, this DNA segment is repeated from 5 to about 40 times. In patients with FXS, the CGG segment is repeated more than 200 times. The abnormally expanded CGG segment turns off (silences) the FMR1 gene, which prevents the gene from producing FMRP. Males and females with 55 to 200 repeats of the CGG segment are said to have an FMR1 gene premutation. Most people with this premutation are intellectually normal. In some cases, however, individuals with a premutation have lower than normal amounts of FMRP. As a result, they may have mild versions of the physical features seen in FXS. FXS is inherited in an X-linked dominant pattern. The inheritance is dominant if one copy of the altered gene in each cell is sufficient to cause the condition. X-linked dominant means that in females (who have two X chromosomes), a mutation in one of the two copies of a gene in each cell is sufficient to cause the disorder. In males (who have only one X chromosome), a mutation in the only copy of a gene in each cell causes the disorder. In most cases, males experience more severe symptoms of the disorder than females.

In some embodiments, the subject may be a human child FXS patient. In some embodiments, the subject may be a male human child FXS patient. Such a child patient may be younger than 16 years. In some examples, a child patient may have an age younger than 12, for example, younger than 10, 8, 6, 4 or 2. In some examples, the child patient is an infant, e.g., younger than 12 months, for example equal to or younger than 6 months. Alternatively, the subject may be a human adolescent patient (e.g., 16-20 years old) or a human adult patient having FXS.

Alternatively or in addition, the FXS patient to be treated in the methods disclosed herein may carry an expanded CGG segment within the FMR1 gene. In some examples, a FXS patient may carry an expanded CGG segment repeated more than 200 times within the FMR1 gene. In some examples, a FXS patient may be a male patient having an X-linked mutation in the FMR1 gene. In some embodiments, patients suspected of having or at risk of having FXS with at least one FMR1 gene permutation may be treated with the methods disclosed herein. Genetic testing can be performed to a candidate subject using routine generation sequencing methods, including, but not limited to, next-generation sequencing, pyrosequencing, Sanger sequencing, whole exome sequencing, whole genome sequencing, and the like.

Alternatively or in addition, one or more of the biomarkers disclosed herein (e.g., EEG) may be used for identifying suitable FXS patients for the treatment disclosed herein.

In any of the methods disclosed herein, an effective amount of the AAV viral particles can be given to a FXS patient to alleviate one or more symptoms associated with FXS. In some instances, symptoms associated with FXS may be behavioral, cognitive neurorehabilitation, or a combination thereof. In some examples, symptoms of FXS can be anxiety-related and perseverative behaviors, social behaviors, learning, memory, or a combination thereof.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Effective amounts can also vary, depending on phenotypic variability among subjects having FXS, and/or the genetic mutations involved. Titers of the AAV viral particles herein may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). Dosages may also vary based on the timing of the administration to a human with FXS. These dosages of AAV vectors may range from about 1×10¹¹ vg/kg, about 1×10¹², about 1×10¹³, about 1×10¹⁴, about 1×10¹⁵, about 1×10¹⁶ or more viral genomes per kilogram body weight in an adult. For a neonate, the dosages of AAV vectors may range from about 1×10¹¹, about 1×10¹², about 3×10¹², about 1×10¹³, about 3×10¹³, about 1×10¹⁴, about 3×10¹⁴, about 1×10¹⁵, about 3×10¹⁵, about 1×10¹⁶, about 3×10¹⁶ or more viral genomes per kilogram body weight. Such an amounts can be determined by those skilled in the art following routine practice, for example, examining blood levels of virus at multiple time points after administration to determine whether the dose is proper.

In some instances, the AAV viral particles may be given to a subject by multiple doses. In some examples, the multiple doses can be administered to the subject consequentially via the same route or via different routes. In other examples, the multiple doses can be administered to the subject simultaneously via different routes, e.g., those disclosed herein.

Conventional methods, known to those of ordinary skill in the art of medicine, can be used to administer the AAV9 particle-containing pharmaceutical composition to the FXS subject. For example, this pharmaceutical composition can also be administered parenterally, e.g., by intravenous injection, intracerebroventricular injection, intra-cisterna magna injection, intra-parenchymal injection, or a combination thereof. In some embodiments, AAV particle-containing pharmaceutical composition can administered to the human patient via at least two administration routes. In some examples, the combination of administration routes may be intracerebroventricular injection and intravenous injection. In some examples, the combination of administration routes may be intrathecal injection and intravenous injection. In some examples, the combination of administration routes may be intra-cisterna magna injection and intravenous injection. In some examples, the combination of administration routes may be intra-parenchymal injection and intravenous injection.

In some embodiments, the subject to be treated by the method described herein may be a human patient who has undergone or is subjecting to another anti-FXS therapy. The prior anti-SFXS therapy may be complete. Alternatively, the anti-FXS therapy may be still ongoing. In other embodiments, the FXS patient may be subject to a combined therapy involving the AAV9 particle therapy disclosed herein and a second anti-FXS therapy. Anti-FXS treatments include, but are not limited to, treatment of behavioral abnormalities, seizures, speech therapy, physical therapy, and so forth. Exemplary anti-FXS treatments include, but are not limited to, treatment comprising a GABA receptor agonist, a PI3K isoform-selective inhibitor, a MMP9 antagonist, or a combination thereof. Additional useful agents and therapies can be found in Physician’s Desk Reference, 59.sup.th edition, (2005), Thomson P D R, Montvale N.J.; Gennaro et al., Eds. Remington’s The Science and Practice of Pharmacy 20.sup.th edition, (2000), Lippincott Williams and Wilkins, Baltimore Md.; Braunwald et al., Eds. Harrison’s Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

In some embodiments, the dosage of the AAV particles such as AAV9 particles or a pharmacological composition thereof may be adjusted based on the FXS patient’s response to the treatment. For example, if the FXS patient shows worsening of one or more behavior features (e.g., behavioral and/or cognitive activities), the dose of the AAV particles can be reduced. Alternatively, if the FXS patient does not show clear improvement of FXSsymptoms, the dose of the AAV particles may be increased. See descriptions below for using behavior features as biomarkers for assessing suitable doses and/or treatment efficacy of AAV9 particles in individual FXS patients.

### IV. Use of EEG and Behavior Feature Biomarkers for Determination of Personalized Doses of AAV9 particles for Individual FXS Patients

In any of the treatment methods disclosed herein, one or more biomarkers disclosed herein may be used for identifying suitable patients, for determining personalized AAV particle dosage, and/or for assessing treatment efficacy. The term “biomarker” as used herein refers to an indicator (one factor or a combination of factors) that provides information about clinical features of a FXS patient, for example, phenotypic severity of the disease, patient responsiveness to the treatment, etc. Exemplary biomarkers include EEG (e.g., long-term potentiation or LTP), one or more behavior features (e.g., agitation, or memory deficit), or a combination thereof. FMRP is a synaptic protein, and its level and/or distribution correlates with levels of neural activity in the brain. Loss of FMRP causes in an increase in the threshold for LTP, which results in aberrant neural activity that can be measured and recorded using EEG. Accordingly, EEG can be used to monitor levels and/or distribution of FMRP, thereby benefiting FXS patient diagnosis and assessment of treatment efficacy.

In some embodiments, long-term potentiation (LTP) patterns assessed by electroencephalogram (EEG) can be used as a biomarker for assessing and determining suitable doses of AAV particles such as AAV9 particles disclosed herein for use in the method of treating FXS. In some examples, after administration of an initial dose of the AAV particles, the LTP pattern of the FXS patient may be monitored using EEG. If the initial dose of the AAV9 particles does not show impact on the LTP pattern of the FXS patient, the dose of the AAV9 particles may be maintained or increased.

In other embodiments, agitation can be used as a biomarker assessing and determining suitable doses of AAV9 particles for use in the method disclosed herein, or for assessing treatment efficacy. Agitation refers to a state of anxiety or nervous excitement displayed as anxiety-related and perseverative behaviors. After administration of an initial dose of the AAV particles development and/or progression of agitation in the FXS patient may be monitored following routine practice or the methods provided herein. If the FXS patient develops agitation, has a progression of agitation, or has an enhanced sensation of anxiety, the dose of the AAV particles can be reduced. Alternatively, if the initial dose of the AAV particles does not lead to development of agitation or alleviates/reduces agitation in the FXS patient, this indicates that the AAV9 particles at the initial dose is effective. The dose of the AAV particles may be maintained or increased.

In other embodiments, memory deficit can be used as a biomarker assessing and determining suitable doses of AAV9 particles for use in the method disclosed herein, or for assessing treatment efficacy. Memory deficit refers to the inability of a FXS patient to learn as displayed by short term memory. After administration of an initial dose of the AAV particles development and/or progression of memory deficit in the FXS patient may be monitored following routine practice or the methods provided herein. If the FXS patient develops memory deficit or has a progression of memory deficit, the dose of the AAV particles can be reduced. Alternatively, if the initial dose of the AAV particles does not lead to development of memory deficit or does not improve memory deficit in the FXS patient, this indicates that the AAV9 particles at the initial dose is effective. The dose of the AAV particles may be maintained or increased.

Using one or more of the EEG and/or behavior feature biomarkers disclosed herein, a suitable dose of the AAV particles may be determined for an individual FXS patient.

The one or more EEG and/or behavior feature biomarkers disclosed herein can also be used to assess therapeutic efficacy of the AAV particles-involving treatment disclosed herein. Such an assessment may help determine further treatment strategy, e.g., continuing the AAV-mediated FMR1 gene therapy, modifying the AAV-mediated FMR1 gene therapy (change dose, dosing interval, etc.), combining the AAV-mediated FMR1 gene therapy with another anti-FXS therapy, or terminate the AAV-mediated FMR1 gene therapy.

### V. Kits for Use in FXS Treatment

The present disclosure also provides kits for use in treating FXS as described herein. A kit for therapeutic use as described herein may include one or more containers comprising the AAV particles such as AAV9 particles as described herein, formulated in a pharmaceutical composition.

In some embodiments, the kit can additionally comprise instructions for use of the AAV particles in any of the methods described herein. The included instructions may comprise a description of administration of the AAV particles or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the rapamycin compound or the pharmaceutical composition comprising such to a subject who has or is suspected of having FXS.

The instructions relating to the use of the AAV particles as described herein generally include information as to dosage, dosing schedule, and route of administration for the intended treatment. In some embodiments, the instructions comprise a description of optimizing the dose of rapamycin in a subject having FXS using one or more of the behavior features as a biomarker, e.g., those described herein. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

In some embodiments, the kit include one or more AAV vectors disclosed herein. In some examples, the kit can additionally comprise one or more helper vectors to be used in combination with the AAV vectors disclosed herein. In some examples, a kit may include a host cell suitable for use with the AAV vectors disclosed herein. A kit can further instructions for use of AAV vectors according to methods as described herein.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B.D. Hames & S.J. Higgins eds.(1985»; Transcription and Translation (B.D. Hames & S.J. Higgins, eds. (1984»; Animal Cell Culture (R.I. Freshney, ed. (1986»; Immobilized Cells and Enzymes (1RL Press, (1986»; and B. Perbal, A practical Guide To Molecular Cloning (1984); F.M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Development of AAV Vectors Expressing FMRP

Fragile X Syndrome (FXS) is a monogenetic syndrome caused by an expansion of CGG repeats in the fragile X mental retardation protein (FMR1) gene which results in the loss of the gene product, the Fragile X mental retardation protein (FMRP), and the leading cause of inherited intellectual disability. As monogenetic disorders are particularly attractive targets for gene therapy in which theoretically correction of a single gene may rescue the entire organism, development of adeno-associated virus (AAV) to restore FMRP expression in patients with FXS can be a useful treatment strategy.

CNS-targeted AAV vectors capable of producing human FMRP (isoform 1) were designed and cloned. Specifically, two different viral vectors expressing FMRP or GFP (green fluorescent protein as a control) were developed: (1) the self-complementary AAV vector (scAAV; circumventing the need for DNA synthesis) (FIG. 1A), as well as (2) regular AAV vector (FIG. 2A). The scAAV vector, scAAV9-CB-FMR1, was based on a scAAV backbone and contained the human FMR1 coding sequence under the control of the hybrid CMV enhancer/beta-actin promoter CB (FIG. 1A). The regular AAV vector, AAV- CAGFMR1, comprises the human FMR1 coding sequence under the control of the CB promoter (a.k.a. CAG promoter) (FIG. 2A). The viruses were generated to confer AAV9 tropism for optimal transduction of forebrain neurons and the FMR1 insert fragment size was about 3 kilobases (kb).

Both vectors were tested in primary hippocampal and/or cortical mouse neurons and were shown to express the full length FMRP protein in a dose-dependent manner. Specifically, primary cultured mouse cortical neurons were transduced at the eighth cell division with 1, 2, 5, or 10 µ1 of scAAV9-CB-FMR1, scAAV9-CB-GFP, or scAAV9-CBflag-FMR1 viral particles. After the 13th cell division, the cells were harvested and subjected to western blot analysis. FIG. 1B shows a dose-dependent expression of both flag-tagged FMR1 and un-tagged FMRPs. Additionally, primary cultured mouse hippocampal neurons were transduced with 3, 1.5, 0.8, or 0.4 viral genomes per ml (vg/ml) of AAV-CAGFMR1 or AAV-CAG-GFP followed by western blot analysis. FIG. 2B shows a dose dependent expression of both FMRP and the control GFP protein in the AAV-CAGFMR1 and AAV-CAG-GFP transduced cells, respectively.

The mRNA expression of FMR1 and GFP was also measured in primary cultured mouse hippocampal neurons that were transduced with 3, 1.5, or 0.3 viral genomes per ml (vg/ml) of AAV-CAG-FMR1 or AAV-CAG-GFP. FIG. 2C shows a dose-dependent expression of both FMR1 mRNA and the control GFP mRNA in the AAV-CB-FMR1 and AAV-CB-GFP transduced cells, respectively.

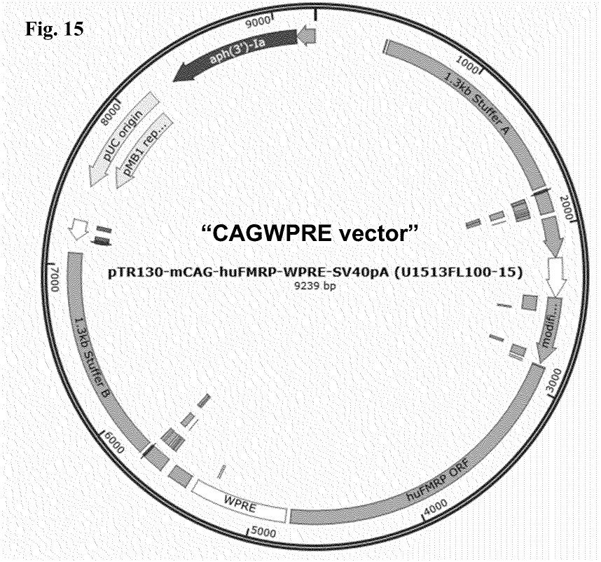

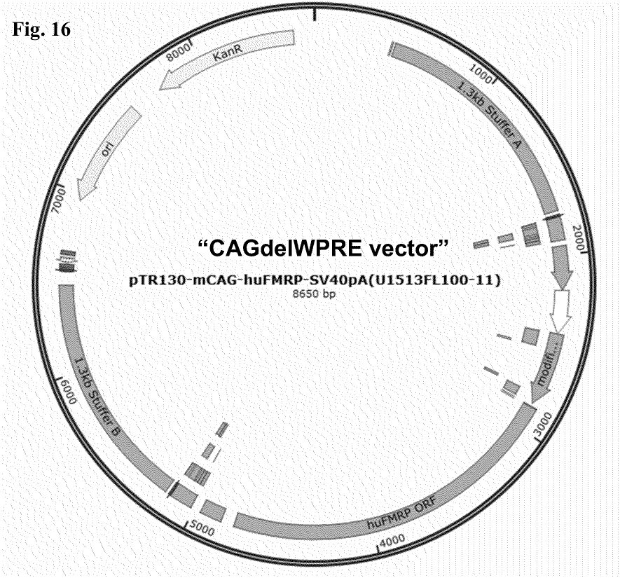

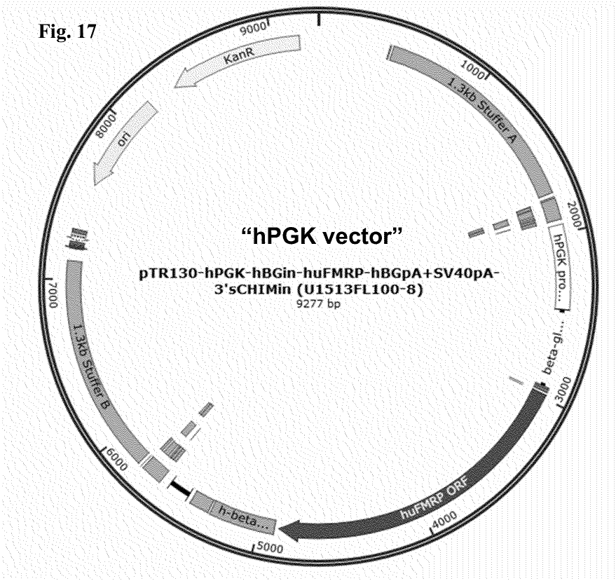

Three additional vectors were developed in order to optimize expression of FMR1 and safety. To construct these additional vectors the FMR1 transgene was cloned into a vector backbone carrying a kanamycin resistance gene. Additionally, the transgene is flanked by stuffer sequences, which reduce the packaging of plasmid backbone with bacterial sequences that otherwise may become packaged. The constructs generated using this vector are as follows: (1) pTR130- mCAG-huFMRP-WPRE-SV40pA (hereinafter “CAGWPRE” vector) (See FIG. 15) (SEQ ID No: 4), which comprises the same transgene as in AAV-CAGFMR1 in the vector backbone described above; (2) pTR130-mCAG-huFMRP-SV40pA (hereinafter “CAGdelWPRE vector”) (See FIG. 16) (SEQ ID No: 5), which lacks the WPRE relative to the CAGWPRE construct, in the vector backbone describe above; and (3) pTR130-hPGK-hBGin-huFMRP-hBGpA+SV40pA-3′sCHIMin (hereinafter “hPGK vector”) (See FIG. 17) (SEQ ID No: 6), which contains an hPGK promoter to drive expression of FMRP, as well as a 3′ hβ-globin poly(A) signal, which acts as an mRNA transcript stabilizer element (See FIG. 17), and a small chimeric intron sequence, in the vector backbone described above. These modifications were chosen to facilitate optimal expression of the transgene in vivo, and also to improve the safety of the constructs.

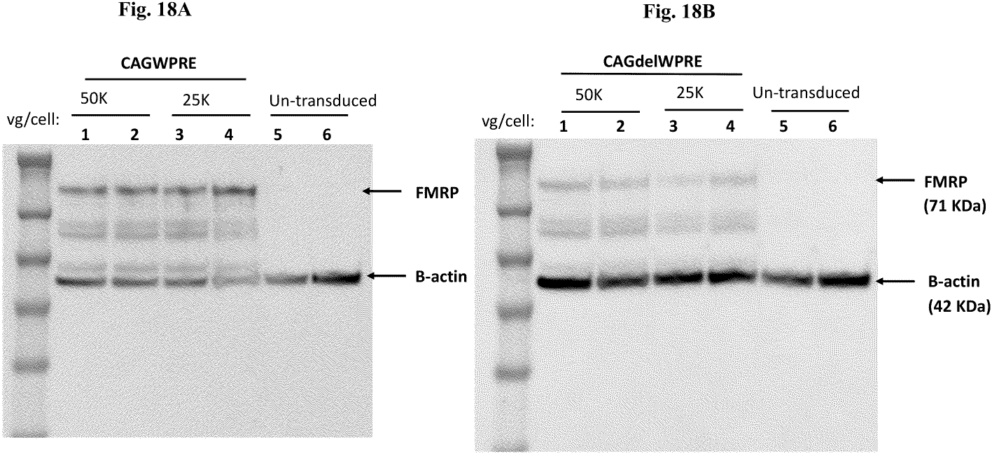

In order to compare the FMRP expression efficiencies of the CAGWPRE and CAGdelWPRE vectors, CHO-Lec2 cells were transduced with the vectors, and expression was evaluated by Western Blot. Cells transduced with the CAGdelWPRE vector expressed FMRP, but the observed expresion was less than that observed in cells transduced with the CAGWPRE vector. FIGS. 18A and 18B.

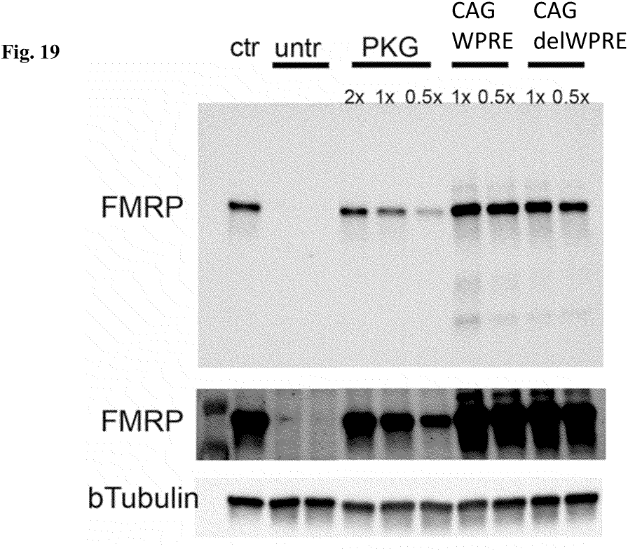

In order to compare the expression efficiency of the hPGK vector to that of the CAGWPRE and CAGdelWPRE vectors in neuronal cells, E17 cultured mouse cortical neurons were transduced with the vectors at DIV14, and were allowed to express the vectors for 5 days before harvesting at DIV19. Harvested neurons were subsequently subjected to Western Blot analysis. Use of the hPGK promoter in the hPGK vector resulted in reduced expression of FRMP in neurons relative to that observed in neurons transduced with CAG-driven vectors. FIG. 19.

TABLE 2

Sequences of Exemplary plasmids

| | | |
|---|---|---|
| SEQ ID: | Description | Sequence |
| 4 | CAGWPRE plasmid | TCGCGCGTTTCGGTGATGACGGTGAAAACCTCTGACACATGCAGC TCCCGGAGACGGTCACAGCTTGTCTGTAAGCGGATGCCGGGAGC AGACAAGCCCGTCAGGGCGCGTCAGCGGGTGTTGGCGGGTGTCG GGGCTGGCTTAACTATGCGGCATCAGAGCAGATTGTACTGAGAG TGCACCATATGCGGTGTGAAATACCGCACAGATGCGTAAGGAGA AAATACCGCATCAGGCGCCATTCGCCATTCAGGCTGCGCAACTGT TGGGAAGGGCGATCGGTGCGGGCCTCTTCGCTATTACGCCAGCT GGCGAAAGGGGGATGTGCTGCAAGGCGATTAAGTTGGGTAACG CCAGGGTTTTCCCAGTCACGACGTTGTAAAACGACGGCCAGAGA ATTCGAGCTCGGTACCTCGCGAATACATCTAGATGTCGACACCGG TGGCCGGCCCAGCCCTGACTGCTATGGACTTTTAGTGCTTGTGCA CTGCTACCAGCCATGCCCTGAGTGCACAGGGTTACCCTGGCTGGG GCCTGGAAGCATATAAAAGAAGCTCAGAGTCACATCCAGGTACA CCTTTCACCTGGGCCAGCTTCATTCAGAAGGCCAGGAGGGGGGG ATTTTGCCATGTCTGAAGTTCCAGTTATAGGCACACAGTTGCATCT TACTAGGTCTGGTCTAGGGATCACCAGTGACTTCCAGCCTCTAATT CCACACTGTTCAGCAGGCACCTGTTGTGGCAGGCTGGTCTTAGTT AGCTAGGGCAGGGTTTCTTCACTGGGAGTCCTGGCATTCAGAATG ATTAAGGGCTCCCTCTGCCTACCTATCCCCAGTCAACCAGCCCCAG GTGTGTAGCTCTAGTCTGAGACTCATATATCCAGGGTCACCTCCTG |
| | | GGATCAAGTGCATGAGCCCAAGCTGGGATCTTTCCCCAGTTACAT ATTTCTGGACAGTAGGGCTCAGAGTCTCCAGTGCCAGTCCTGTTT CAGAGTATGGAAAAAGTAACCATTGTTACAGCCACAGTCCTTGGG TTAGCCCTAACTCAGCTAATCCAGCAGGCCCAAGGACACCTGAAT ATCCAAGGTATGAGTGTGAGGCCCTGTAAGGTAATTCTAGCCCCT TATGCCTAACTTGATTATCAAGACCAAGCTATATCAGGGATGAAC CAGAGGCTCCATGACACCCCAGCCACCTAGCTAAACTTGGGGGTT GGGTACAAGTTAGCCCAGAACATACCTTATAGAATTGCCTCTCTA GGGTGAGCAAGGGCCAACCTGCCTATTTGCCCTCTCACCCCCATT GCAATAGCTTTGGCTCCCAGTACCTCTTCCCTGGCTTCATTAGCAG ATGGCACCCAGCAGATAAAGGTCTATACCCCTGACAAGGGAAAC ATGGAAAGTATCAGGACCAACATGGTCCACAGCAGAAGTGTCTG GAGTCCATCCTGCATGGCCTTGAGTCCAGGCACAGGAGTCTCCAG TGAGGGTAAACCCCAATCATTGTCCATCCAGGTTTTGCCATAAGA CTTGGGCCAGGGTAGCTAAAGCAGATTTACCCCTGCAAGGAGAC ACCTCATTGGAAACTGAAAGAGACTCCCCACCAGCTTGAAAGGCC AGTCATGCTTTTGCCTGACTCCTGCTCTCTATGCAGTGGCAATCTA AGTGGGAGGTCTGTTCTTCCCAAGAGAGGACCAAGTTTCTGTCCC AAGGCAATAATCCTGTTATCATTGGCTCCTAGCTGCCATTGTTCTG ATTGAGGGTTTAAACTCCGGAATTTAAATCCTGCAGGCAGCTGCG CGCTCGCTCGCTCACTGAGGCCGCCCGGGCAAAGCCCGGGCGTC GGGCGACCTTTGGTCGCCCGGCCTCAGTGAGCGAGCGAGCGCGC AGAGAGGGAGTGGCCAACTCCATCACTAGGGGTTCCTTGCAGGC CTCCTAGGCTTGCATGCAGTACTATTGACGTCAATAATGACGTAT GTTCCCATAGTAACGCCAATAGGGACTTTCCATTGACGTCAATGG GTGGAGTATTTACGGTAAACTGCCCACTTGGCAGTACATCAAGTG TATCATATGCCAAGTACGCCCCCTATTGACGTCAATGACGGTAAA TGGCCCGCCTGGCATTATGCCCAGTACATGACCTTATGGGACTTTC CTACTTGGCAGTACATCTACGTATTAGTCATCGCTATTACCATGGT CGAGGTGAGCCCCACGTTCTGCTTCACTCTCCCCATCTCCCCCCCC TCCCCACCCCCAATTTTGTATTTATTTATTTTTTAATTATTTTGTGCA GCGATGGGGGCGGGGGGGGGGGGGGGGGGGCGGGGCGAGGC GGAGAGGTGCGGCGGCAGCCAATCAGAGCGGCGCGCTCCGAAA GTTTCCTTTTATGGCGAGGCGGCGGCGGCGGCGGCCCTATAAAA AGCGAAGCGCGCGGCGGGCGGGAGTCGCTGCGCGCTGCCTTCGC CCCGTGCCCCGCTCCGCCGCCGCCTCGCGCCGCCCGCCCCGGCTC TGACTGACCGCGTTACTCCCACAGGTGAGCGGGCGGGACGGCCC TTCTCCTCCGGGCTGTAATTAGCGCTTGGTTTAATGACGGCTTGTT TCTTTTCTGTGGCTGCGTGAAAGCCTTGAGGGGCTCCGGGAGGG CCCTTTGTGCGGGGGGAGCGGCTCGGGGCTGTCCGCGGGGGGA CGGCTGCCTTCGGGGGGGACGGGGCAGGGCGGGGTTCGGCTTC TGGCGTGTGACCGGCGGCTCTAGAGCCTCTGCTAACCATGTTCAT GCCTTCTTCTTTTTCCTACAGCTCCTGGGCAACGTGCTGGTTATTG TGCTGTCTCATCATTTTGGCAAAGAATTGGATCCCCGGGTACCGG TGCCACCATGGAGGAGCTGGTGGTGGAAGTGCGGGGCTCCAATG GCGCTTTCTACAAGGCATTTGTAAAGGATGTTCATGAAGATTCAA TAACAGTTGCATTTGAAAACAACTGGCAGCCTGATAGGCAGATTC |
| | | CATTTCATGATGTCAGATTCCCACCTCCTGTAGGTTATAATAAAGA TATAAATGAAAGTGATGAAGTTGAGGTGTATTCCAGAGCAAATG AAAAAGAGCCTTGCTGTTGGTGGTTAGCTAAAGTGAGGATGATA AAGGGTGAGTTTTATGTGATAGAATATGCAGCATGTGATGCAACT TACAATGAAATTGTCACAATTGAACGTCTAAGATCTGTTAATCCCA ACAAACCTGCCACAAAAGATACTTTCCATAAGATCAAGCTGGATG TGCCAGAAGACTTACGGCAAATGTGTGCCAAAGAGGCGGCACAT AAGGATTTTAAAAAGGCAGTTGGTGCCTTTTCTGTAACTTATGATC CAGAAAATTATCAGCTTGTCATTTTGTCCATCAATGAAGTCACCTC AAAGCGAGCACATATGCTGATTGACATGCACTTTCGGAGTCTGCG CACTAAGTTGTCTCTGATAATGAGAAATGAAGAAGCTAGTAAGCA GCTGGAGAGTTCAAGGCAGCTTGCCTCGAGATTTCATGAACAGTT TATCGTAAGAGAAGATCTGATGGGTCTAGCTATTGGTACTCATGG TGCTAATATTCAGCAAGCTAGAAAAGTACCTGGGGTCACTGCTAT TGATCTAGATGAAGATACCTGCACATTTCATATTTATGGAGAGGA TCAGGATGCAGTGAAAAAAGCTAGAAGCTTTCTCGAATTTGCTGA AGATGTAATACAAGTTCCAAGGAACTTAGTAGGCAAAGTAATAG GAAAAAATGGAAAGCTGATTCAGGAGATTGTGGACAAGTCAGGA GTTGTGAGGGTGAGGATTGAGGCTGAAAATGAGAAAAATGTTCC ACAAGAAGAGGAAATTATGCCACCAAATTCCCTTCCTTCCAATAAT TCAAGGGTTGGACCTAATGCCCCAGAAGAAAAAAAACATTTAGAT ATAAAGGAAAACAGCACCCATTTTTCTCAACCTAACAGTACAAAA GTCCAGAGGGTGTTAGTGGCTTCATCAGTTGTAGCAGGGGAATC CCAGAAACCTGAACTCAAGGCTTGGCAGGGTATGGTACCATTTGT TTTTGTGGGAACAAAGGACAGCATCGCTAATGCCACTGTTCTTTT GGATTATCACCTGAACTATTTAAAGGAAGTAGACCAGTTGCGTTT GGAGAGATTACAAATTGATGAGCAGTTGCGACAGATTGGAGCTA GTTCTAGACCACCACCAAATCGTACAGATAAGGAAAAAAGCTATG TGACTGATGATGGTCAAGGAATGGGTCGAGGTAGTAGACCTTAC AGAAATAGGGGGCACGGCAGACGCGGTCCTGGATATACTTCAGG AACTAATTCTGAAGCATCAAATGCTTCTGAAACAGAATCTGACCA CAGAGACGAACTCAGTGATTGGTCATTAGCTCCAACAGAGGAAG AGAGGGAGAGCTTCCTGCGCAGAGGAGACGGACGGCGGCGTGG AGGGGGAGGAAGAGGACAAGGAGGAAGAGGACGTGGAGGAGG CTTCAAAGGAAACGACGATCACTCCCGAACAGATAATCGTCCACG TAATCCAAGAGAGGCTAAAGGAAGAACAACAGATGGATCCCTTC AGATCAGAGTTGACTGCAATAATGAAAGGAGTGTCCACACTAAA ACATTACAGAATACCTCCAGTGAAGGTAGTCGGCTGCGCACGGG TAAAGATCGTAACCAGAAGAAAGAGAAGCCAGACAGCGTGGATG GTCAGCAACCACTCGTGAATGGAGTACCCTGATAAGAATTCGATA TCAAGCTTATCGATAATCAACCTCTGGATTACAAAATTTGTGAAAG ATTGACTGGTATTCTTAACTATGTTGCTCCTTTTACGCTATGTGGA TACGCTGCTTTAATGCCTTTGTATCATGCTATTGCTTCCCGTATGG CTTTCATTTTCTCCTCCTTGTATAAATCCTGGTTGCTGTCTCTTTATG AGGAGTTGTGGCCCGTTGTCAGGCAACGTGGCGTGGTGTGCACT GTGTTTGCTGACGCAACCCCCACTGGTTGGGGCATTGCCACCACC TGTCAGCTCCTTTCCGGGACTTTCGCTTTCCCCCTCCCTATTGCCAC |
| | | GGCGGAACTCATCGCCGCCTGCCTTGCCCGCTGCTGGACAGGGG CTCGGCTGTTGGGCACTGACAATTCCGTGGTGTTGTCGGGGAAAT CATCGTCCTTTCCTTGGCTGCTCGCCTGTGTTGCCACCTGGATTCT GCGCGGGACGTCCTTCTGCTACGTCCCTTCGGCCCTCAATCCAGC GGACCTTCCTTCCCGCGGCCTGCTGCCGGCTCTGCGGCCTCTTCCG CGTCTTCGCCTTCGCCCTCAGACGAGTCGGATCTCCCTTTGGGCCG CCTCCCCGCATCGATACCGTCGACCCGGGCGGCCGCTTCGAGCAG ACATGATAAGATACATTGATGAGTTTGGACAAACCACAACTAGAA TGCAGTGAAAAAAATGCTTTATTTGTGAAATTTGTGATGCTATTGC TTTATTTGTAACCATTATAAGCTGCAATAAACAAGTTAGATCTGTG TGTTGGTTTTTTCTTAAGGTGTGATTAATGAGCTACCAGGTCTCGA GGAGGAACCCCTAGTGATGGAGTTGGCCACTCCCTCTCTGCGCGC TCGCTCGCTCACTGAGGCCGGGCGACCAAAGGTCGCCCGACGCC CGGGCTTTGCCCGGGCGGCCTCAGTGAGCGAGCGAGCGCGCAGC TGCCTGCAGGTTATAACCTGCAGGTTAATTAAGCCTTGTAGTCTA GCCAGGGTGTATAACCCCTCCAGCCCATGTTCAAAGAGCTGTCTT GCCTAGCCAGTTGCACATACAAAGTGATAAATGGGAGCTGGCATT GTGCCCTTGGAGGCACTCTGAAGGATCACCAGTGAACCCAGCAG CAAAGATACATAGGCTCTTAATTCATGCCAGGATCTCAGAGAGGC AATGGCTTGCACAATCAGGGGTTATTCTGACAGCATGAACTCTGG TGCCAGACAATTTTATGTATCAGGCAATGTGCATTACTTGAGGTG GATTACAGACCCAGTGAGTAACCCAGGACCAGGAGTAAACAGGC CCTAATCCCAGCTTGATTGACACCAGGCTTGAAGATCCTTACCATT ATGAAAACAGCACATGGTCTGTTGACAATTACTTGTAGCATAGGT AGCCCAGGCAGAGTGGCAGACAGGGTACTGATAGTAGTTAGAGA ACCTCCCAGATAAGCTACCTGACCTTCTCTAATCTTGAGTTCTGTG GGCAACCAGCCCAGTGAGCATCTTGGGTTCTTGGAAATCCAGACC CTACTCACCTGAGTATAAATGGGGCAGCCACCCACCCAGACTGAT CCATCCTACCTTTGAGGCTACCCATGAGGTAATTAGGCCAGAATG TGAAAGGGAGGAGCCCAGAGCATTGTTCCTTGTATTACCATTGGG AACCTTGTTAGATGGGGAGGAATGCACTTCAGCCACCCTAGAGG AGTTGAGTCATTAGAAGAAGAAGGCTGCTTCCCCATCAGGAGAA GATCCAGCCAGTAAGTATATAGGTTACAACTGCCAGGTACTATGG GCTTCTCCAGACCCTTCCTACCCAGGAACTAGAAGGTTGGAGCCT AAAGTCCCCTACCCATGTGCTGACTGATCCAGAGTTACACTCCCTC AGACTCATCCTCCAGACAGGGTTCCCAGTTATTAGGATTGCAATA ACCATCCAGTTCCCAAACTATTCCAGCTTCCTATCCAGTAATAAGC CCTTATTCTTTAACCTCTGAAGAAACCCTGAGTGAACAGGCTGTGC AGGGCTCCTGTCACTTCTGTCAGCCCAGATAGGTATGAAATCTCTT CAAATGTATTGCCAATGACATTGCCAGGCACAGATTCTCCCAGTT ACCCAACCAGGAACACCAGCTAGTGTCAAACAGTCAATGCCCTTC TACCAGCCATTTGAGACACTACCAGGCAGGCAAAGCCAAGTGGC TTCACTCCTTATTTATATTAGCTCAGAGGAGCCATCTAACAGCTCT TACTCAACTAGACTAGCTGCATGAACAGTGTACAGCTAGCTGCGC AATCGGATCCCGGGCCCGTCGACTGCAGAGGCCTGCATGCAAGC TTGGTGTAATCATGGTCATAGCTGTTTCCTGTGTGAAATTGTTATC CGCTCACAATTCCACACAACATACGAGCCGGAAGCATAAAGTGTA |
| | | AAGCCTGGGGTGCCTAATGAGTGAGCTAACTCACATTAATTGCGT TGCGCTCACTGCCCGCTTTCCAGTCGGGAAACCTGTCGTGCCAGC TGCATTAATGAATCGGCCAACGCGCGGGGAGAGGCGGTTTGCGT ATTGGGCGCTCTTCCGCTTCCTCGCTCACTGACTCGCTGCGCTCGG TCGTTCGGCTGCGGCGAGCGGTATCAGCTCACTCAAAGGCGGTA ATACGGTTATCCACAGAATCAGGGGATAACGCAGGAAAGAACAT GTGAGCAAAAGGCCAGCAAAAGGCCAGGAACCGTAAAAAGGCC GCGTTGCTGGCGTTTTTCCATAGGCTCCGCCCCCCTGACGAGCATC ACAAAAATCGACGCTCAAGTCAGAGGTGGCGAAACCCGACAGGA CTATAAAGATACCAGGCGTTTCCCCCTGGAAGCTCCCTCGTGCGC TCTCCTGTTCCGACCCTGCCGCTTACCGGATACCTGTCCGCCTTTCT CCCTTCGGGAAGCGTGGCGCTTTCTCATAGCTCACGCTGTAGGTA TCTCAGTTCGGTGTAGGTCGTTCGCTCCAAGCTGGGCTGTGTGCA CGAACCCCCCGTTCAGCCCGACCGCTGCGCCTTATCCGGTAACTAT CGTCTTGAGTCCAACCCGGTAAGACACGACTTATCGCCACTGGCA GCAGCCACTGGTAACAGGATTAGCAGAGCGAGGTATGTAGGCGG TGCTACAGAGTTCTTGAAGTGGTGGCCTAACTACGGCTACACTAG AAGAACAGTATTTGGTATCTGCGCTCTGCTGAAGCCAGTTACCTTC GGAAAAAGAGTTGGTAGCTCTTGATCCGGCAAACAAACCACCGC TGGTAGCGGTGGTTTTTTTGTTTGCAAGCAGCAGATTACGCGCAG AAAAAAAGGATCTCAAGAAGATCCTTTGATCTTTTCTACGGGGTC TGACGCTCAGTGGAACGAAAACTCACGTTAAGGGATTTTGGTCAT GAGATTATCAAAAAGGATCTTCACCTAGATCCTTTTAAATTAAAAA TGAAGTTTTAAATCAAGCCCAATCTGAATAATGTTACAACCAATTA ACCAATTCTGATTAGAAAAACTCATCGAGCATCAAATGAAACTGC AATTTATTCATATCAGGATTATCAATACCATATTTTTGAAAAAGCC GTTTCTGTAATGAAGGAGAAAACTCACCGAGGCAGTTCCATAGG ATGGCAAGATCCTGGTATCGGTCTGCGATTCCGACTCGTCCAACA TCAATACAACCTATTAATTTCCCCTCGTCAAAAATAAGGTTATCAA GTGAGAAATCACCATGAGTGACGACTGAATCCGGTGAGAATGGC AAAAGTTTATGCATTTCTTTCCAGACTTGTTCAACAGGCCAGCCAT TACGCTCGTCATCAAAATCACTCGCATCAACCAAACCGTTATTCAT TCGTGATTGCGCCTGAGCGAGACGAAATACGCGATCGCTGTTAAA AGGACAATTACAAACAGGAATCGAATGCAACCGGCGCAGGAACA CTGCCAGCGCATCAACAATATTTTCACCTGAATCAGGATATTCTTC TAATACCTGGAATGCTGTTTTTCCGGGGATCGCAGTGGTGAGTAA CCATGCATCATCAGGAGTACGGATAAAATGCTTGATGGTCGGAA GAGGCATAAATTCCGTCAGCCAGTTTAGTCTGACCATCTCATCTGT AACATCATTGGCAACGCTACCTTTGCCATGTTTCAGAAACAACTCT GGCGCATCGGGCTTCCCATACAAGCGATAGATTGTCGCACCTGAT TGCCCGACATTATCGCGAGCCCATTTATACCCATATAAATCAGCAT CCATGTTGGAATTTAATCGCGGCCTCGACGTTTCCCGTTGAATATG GCTCATAACACCCCTTGTATTACTGTTTATGTAAGCAGACAGTTTT ATTGTTCATGATGATATATTTTTATCTTGTGCAATGTAACATCAGA GATTTTGAGACACGGGCCAGAGCTGCA |
| 5 | CAGdelWPRE plasmid | TCGCGCGTTTCGGTGATGACGGTGAAAACCTCTGACACATGCAGC TCCCGGAGACGGTCACAGCTTGTCTGTAAGCGGATGCCGGGAGC |
| | | AGACAAGCCCGTCAGGGCGCGTCAGCGGGTGTTGGCGGGTGTCG GGGCTGGCTTAACTATGCGGCATCAGAGCAGATTGTACTGAGAG TGCACCATATGCGGTGTGAAATACCGCACAGATGCGTAAGGAGA AAATACCGCATCAGGCGCCATTCGCCATTCAGGCTGCGCAACTGT TGGGAAGGGCGATCGGTGCGGGCCTCTTCGCTATTACGCCAGCT GGCGAAAGGGGGATGTGCTGCAAGGCGATTAAGTTGGGTAACG CCAGGGTTTTCCCAGTCACGACGTTGTAAAACGACGGCCAGAGA ATTCGAGCTCGGTACCTCGCGAATACATCTAGATGTCGACACCGG TGGCCGGCCCAGCCCTGACTGCTATGGACTTTTAGTGCTTGTGCA CTGCTACCAGCCATGCCCTGAGTGCACAGGGTTACCCTGGCTGGG GCCTGGAAGCATATAAAAGAAGCTCAGAGTCACATCCAGGTACA CCTTTCACCTGGGCCAGCTTCATTCAGAAGGCCAGGAGGGGGGG ATTTTGCCATGTCTGAAGTTCCAGTTATAGGCACACAGTTGCATCT TACTAGGTCTGGTCTAGGGATCACCAGTGACTTCCAGCCTCTAATT CCACACTGTTCAGCAGGCACCTGTTGTGGCAGGCTGGTCTTAGTT AGCTAGGGCAGGGTTTCTTCACTGGGAGTCCTGGCATTCAGAATG ATTAAGGGCTCCCTCTGCCTACCTATCCCCAGTCAACCAGCCCCAG GTGTGTAGCTCTAGTCTGAGACTCATATATCCAGGGTCACCTCCTG GGATCAAGTGCATGAGCCCAAGCTGGGATCTTTCCCCAGTTACAT ATTTCTGGACAGTAGGGCTCAGAGTCTCCAGTGCCAGTCCTGTTT CAGAGTATGGAAAAAGTAACCATTGTTACAGCCACAGTCCTTGGG TTAGCCCTAACTCAGCTAATCCAGCAGGCCCAAGGACACCTGAAT ATCCAAGGTATGAGTGTGAGGCCCTGTAAGGTAATTCTAGCCCCT TATGCCTAACTTGATTATCAAGACCAAGCTATATCAGGGATGAAC CAGAGGCTCCATGACACCCCAGCCACCTAGCTAAACTTGGGGGTT GGGTACAAGTTAGCCCAGAACATACCTTATAGAATTGCCTCTCTA GGGTGAGCAAGGGCCAACCTGCCTATTTGCCCTCTCACCCCCATT GCAATAGCTTTGGCTCCCAGTACCTCTTCCCTGGCTTCATTAGCAG ATGGCACCCAGCAGATAAAGGTCTATACCCCTGACAAGGGAAAC ATGGAAAGTATCAGGACCAACATGGTCCACAGCAGAAGTGTCTG GAGTCCATCCTGCATGGCCTTGAGTCCAGGCACAGGAGTCTCCAG TGAGGGTAAACCCCAATCATTGTCCATCCAGGTTTTGCCATAAGA CTTGGGCCAGGGTAGCTAAAGCAGATTTACCCCTGCAAGGAGAC ACCTCATTGGAAACTGAAAGAGACTCCCCACCAGCTTGAAAGGCC AGTCATGCTTTTGCCTGACTCCTGCTCTCTATGCAGTGGCAATCTA AGTGGGAGGTCTGTTCTTCCCAAGAGAGGACCAAGTTTCTGTCCC AAGGCAATAATCCTGTTATCATTGGCTCCTAGCTGCCATTGTTCTG ATTGAGGGTTTAAACTCCGGAATTTAAATCCTGCAGGCAGCTGCG CGCTCGCTCGCTCACTGAGGCCGCCCGGGCAAAGCCCGGGCGTC GGGCGACCTTTGGTCGCCCGGCCTCAGTGAGCGAGCGAGCGCGC AGAGAGGGAGTGGCCAACTCCATCACTAGGGGTTCCTTGCAGGC CTCCTAGGCTTGCATGCAGTACTATTGACGTCAATAATGACGTAT GTTCCCATAGTAACGCCAATAGGGACTTTCCATTGACGTCAATGG GTGGAGTATTTACGGTAAACTGCCCACTTGGCAGTACATCAAGTG TATCATATGCCAAGTACGCCCCCTATTGACGTCAATGACGGTAAA TGGCCCGCCTGGCATTATGCCCAGTACATGACCTTATGGGACTTTC CTACTTGGCAGTACATCTACGTATTAGTCATCGCTATTACCATGGT |
| | | CGAGGTGAGCCCCACGTTCTGCTTCACTCTCCCCATCTCCCCCCCC TCCCCACCCCCAATTTTGTATTTATTTATTTTTTAATTATTTTGTGCA GCGATGGGGGCGGGGGGGGGGGGGGGGGGGCGGGGCGAGGC GGAGAGGTGCGGCGGCAGCCAATCAGAGCGGCGCGCTCCGAAA GTTTCCTTTTATGGCGAGGCGGCGGCGGCGGCGGCCCTATAAAA AGCGAAGCGCGCGGCGGGCGGGAGTCGCTGCGCGCTGCCTTCGC CCCGTGCCCCGCTCCGCCGCCGCCTCGCGCCGCCCGCCCCGGCTC TGACTGACCGCGTTACTCCCACAGGTGAGCGGGCGGGACGGCCC TTCTCCTCCGGGCTGTAATTAGCGCTTGGTTTAATGACGGCTTGTT TCTTTTCTGTGGCTGCGTGAAAGCCTTGAGGGGCTCCGGGAGGG CCCTTTGTGCGGGGGGAGCGGCTCGGGGCTGTCCGCGGGGGGA CGGCTGCCTTCGGGGGGGACGGGGCAGGGCGGGGTTCGGCTTC TGGCGTGTGACCGGCGGCTCTAGAGCCTCTGCTAACCATGTTCAT GCCTTCTTCTTTTTCCTACAGCTCCTGGGCAACGTGCTGGTTATTG TGCTGTCTCATCATTTTGGCAAAGAATTGGATCCCCGGGTACCGG TGCCACCATGGAGGAGCTGGTGGTGGAAGTGCGGGGCTCCAATG GCGCTTTCTACAAGGCATTTGTAAAGGATGTTCATGAAGATTCAA TAACAGTTGCATTTGAAAACAACTGGCAGCCTGATAGGCAGATTC CATTTCATGATGTCAGATTCCCACCTCCTGTAGGTTATAATAAAGA TATAAATGAAAGTGATGAAGTTGAGGTGTATTCCAGAGCAAATG AAAAAGAGCCTTGCTGTTGGTGGTTAGCTAAAGTGAGGATGATA AAGGGTGAGTTTTATGTGATAGAATATGCAGCATGTGATGCAACT TACAATGAAATTGTCACAATTGAACGTCTAAGATCTGTTAATCCCA ACAAACCTGCCACAAAAGATACTTTCCATAAGATCAAGCTGGATG TGCCAGAAGACTTACGGCAAATGTGTGCCAAAGAGGCGGCACAT AAGGATTTTAAAAAGGCAGTTGGTGCCTTTTCTGTAACTTATGATC CAGAAAATTATCAGCTTGTCATTTTGTCCATCAATGAAGTCACCTC AAAGCGAGCACATATGCTGATTGACATGCACTTTCGGAGTCTGCG CACTAAGTTGTCTCTGATAATGAGAAATGAAGAAGCTAGTAAGCA GCTGGAGAGTTCAAGGCAGCTTGCCTCGAGATTTCATGAACAGTT TATCGTAAGAGAAGATCTGATGGGTCTAGCTATTGGTACTCATGG TGCTAATATTCAGCAAGCTAGAAAAGTACCTGGGGTCACTGCTAT TGATCTAGATGAAGATACCTGCACATTTCATATTTATGGAGAGGA TCAGGATGCAGTGAAAAAAGCTAGAAGCTTTCTCGAATTTGCTGA AGATGTAATACAAGTTCCAAGGAACTTAGTAGGCAAAGTAATAG GAAAAAATGGAAAGCTGATTCAGGAGATTGTGGACAAGTCAGGA GTTGTGAGGGTGAGGATTGAGGCTGAAAATGAGAAAAATGTTCC ACAAGAAGAGGAAATTATGCCACCAAATTCCCTTCCTTCCAATAAT TCAAGGGTTGGACCTAATGCCCCAGAAGAAAAAAAACATTTAGAT ATAAAGGAAAACAGCACCCATTTTTCTCAACCTAACAGTACAAAA GTCCAGAGGGTGTTAGTGGCTTCATCAGTTGTAGCAGGGGAATC CCAGAAACCTGAACTCAAGGCTTGGCAGGGTATGGTACCATTTGT TTTTGTGGGAACAAAGGACAGCATCGCTAATGCCACTGTTCTTTT GGATTATCACCTGAACTATTTAAAGGAAGTAGACCAGTTGCGTTT GGAGAGATTACAAATTGATGAGCAGTTGCGACAGATTGGAGCTA GTTCTAGACCACCACCAAATCGTACAGATAAGGAAAAAAGCTATG TGACTGATGATGGTCAAGGAATGGGTCGAGGTAGTAGACCTTAC |
| | | AGAAATAGGGGGCACGGCAGACGCGGTCCTGGATATACTTCAGG AACTAATTCTGAAGCATCAAATGCTTCTGAAACAGAATCTGACCA CAGAGACGAACTCAGTGATTGGTCATTAGCTCCAACAGAGGAAG AGAGGGAGAGCTTCCTGCGCAGAGGAGACGGACGGCGGCGTGG AGGGGGAGGAAGAGGACAAGGAGGAAGAGGACGTGGAGGAGG CTTCAAAGGAAACGACGATCACTCCCGAACAGATAATCGTCCACG TAATCCAAGAGAGGCTAAAGGAAGAACAACAGATGGATCCCTTC AGATCAGAGTTGACTGCAATAATGAAAGGAGTGTCCACACTAAA ACATTACAGAATACCTCCAGTGAAGGTAGTCGGCTGCGCACGGG TAAAGATCGTAACCAGAAGAAAGAGAAGCCAGACAGCGTGGATG GTCAGCAACCACTCGTGAATGGAGTACCCTGATAAGAATTCGATA TCAAGCTTATCGATATCGATACCGTCGACCCGGGCGGCCGCTTCG AGCAGACATGATAAGATACATTGATGAGTTTGGACAAACCACAAC TAGAATGCAGTGAAAAAAATGCTTTATTTGTGAAATTTGTGATGC TATTGCTTTATTTGTAACCATTATAAGCTGCAATAAACAAGTTAGA TCTGTGTGTTGGTTTTTTCTTAAGGTGTGATTAATGAGCTACCAGG TCTCGAGGAGGAACCCCTAGTGATGGAGTTGGCCACTCCCTCTCT GCGCGCTCGCTCGCTCACTGAGGCCGGGCGACCAAAGGTCGCCC GACGCCCGGGCTTTGCCCGGGCGGCCTCAGTGAGCGAGCGAGCG CGCAGCTGCCTGCAGGTTATAACCTGCAGGTTAATTAAGCCTTGT AGTCTAGCCAGGGTGTATAACCCCTCCAGCCCATGTTCAAAGAGC TGTCTTGCCTAGCCAGTTGCACATACAAAGTGATAAATGGGAGCT GGCATTGTGCCCTTGGAGGCACTCTGAAGGATCACCAGTGAACCC AGCAGCAAAGATACATAGGCTCTTAATTCATGCCAGGATCTCAGA GAGGCAATGGCTTGCACAATCAGGGGTTATTCTGACAGCATGAA CTCTGGTGCCAGACAATTTTATGTATCAGGCAATGTGCATTACTTG AGGTGGATTACAGACCCAGTGAGTAACCCAGGACCAGGAGTAAA CAGGCCCTAATCCCAGCTTGATTGACACCAGGCTTGAAGATCCTT ACCATTATGAAAACAGCACATGGTCTGTTGACAATTACTTGTAGC ATAGGTAGCCCAGGCAGAGTGGCAGACAGGGTACTGATAGTAGT TAGAGAACCTCCCAGATAAGCTACCTGACCTTCTCTAATCTTGAGT TCTGTGGGCAACCAGCCCAGTGAGCATCTTGGGTTCTTGGAAATC CAGACCCTACTCACCTGAGTATAAATGGGGCAGCCACCCACCCAG ACTGATCCATCCTACCTTTGAGGCTACCCATGAGGTAATTAGGCC AGAATGTGAAAGGGAGGAGCCCAGAGCATTGTTCCTTGTATTACC ATTGGGAACCTTGTTAGATGGGGAGGAATGCACTTCAGCCACCCT AGAGGAGTTGAGTCATTAGAAGAAGAAGGCTGCTTCCCCATCAG GAGAAGATCCAGCCAGTAAGTATATAGGTTACAACTGCCAGGTA CTATGGGCTTCTCCAGACCCTTCCTACCCAGGAACTAGAAGGTTG GAGCCTAAAGTCCCCTACCCATGTGCTGACTGATCCAGAGTTACA CTCCCTCAGACTCATCCTCCAGACAGGGTTCCCAGTTATTAGGATT GCAATAACCATCCAGTTCCCAAACTATTCCAGCTTCCTATCCAGTA ATAAGCCCTTATTCTTTAACCTCTGAAGAAACCCTGAGTGAACAG GCTGTGCAGGGCTCCTGTCACTTCTGTCAGCCCAGATAGGTATGA AATCTCTTCAAATGTATTGCCAATGACATTGCCAGGCACAGATTCT CCCAGTTACCCAACCAGGAACACCAGCTAGTGTCAAACAGTCAAT GCCCTTCTACCAGCCATTTGAGACACTACCAGGCAGGCAAAGCCA |
| | | AGTGGCTTCACTCCTTATTTATATTAGCTCAGAGGAGCCATCTAAC AGCTCTTACTCAACTAGACTAGCTGCATGAACAGTGTACAGCTAG CTGCGCAATCGGATCCCGGGCCCGTCGACTGCAGAGGCCTGCAT GCAAGCTTGGTGTAATCATGGTCATAGCTGTTTCCTGTGTGAAATT GTTATCCGCTCACAATTCCACACAACATACGAGCCGGAAGCATAA AGTGTAAAGCCTGGGGTGCCTAATGAGTGAGCTAACTCACATTAA TTGCGTTGCGCTCACTGCCCGCTTTCCAGTCGGGAAACCTGTCGT GCCAGCTGCATTAATGAATCGGCCAACGCGCGGGGAGAGGCGGT TTGCGTATTGGGCGCTCTTCCGCTTCCTCGCTCACTGACTCGCTGC GCTCGGTCGTTCGGCTGCGGCGAGCGGTATCAGCTCACTCAAAG GCGGTAATACGGTTATCCACAGAATCAGGGGATAACGCAGGAAA GAACATGTGAGCAAAAGGCCAGCAAAAGGCCAGGAACCGTAAA AAGGCCGCGTTGCTGGCGTTTTTCCATAGGCTCCGCCCCCCTGAC GAGCATCACAAAAATCGACGCTCAAGTCAGAGGTGGCGAAACCC GACAGGACTATAAAGATACCAGGCGTTTCCCCCTGGAAGCTCCCT CGTGCGCTCTCCTGTTCCGACCCTGCCGCTTACCGGATACCTGTCC GCCTTTCTCCCTTCGGGAAGCGTGGCGCTTTCTCATAGCTCACGCT GTAGGTATCTCAGTTCGGTGTAGGTCGTTCGCTCCAAGCTGGGCT GTGTGCACGAACCCCCCGTTCAGCCCGACCGCTGCGCCTTATCCG GTAACTATCGTCTTGAGTCCAACCCGGTAAGACACGACTTATCGC CACTGGCAGCAGCCACTGGTAACAGGATTAGCAGAGCGAGGTAT GTAGGCGGTGCTACAGAGTTCTTGAAGTGGTGGCCTAACTACGG CTACACTAGAAGAACAGTATTTGGTATCTGCGCTCTGCTGAAGCC AGTTACCTTCGGAAAAAGAGTTGGTAGCTCTTGATCCGGCAAACA AACCACCGCTGGTAGCGGTGGTTTTTTTGTTTGCAAGCAGCAGAT TACGCGCAGAAAAAAAGGATCTCAAGAAGATCCTTTGATCTTTTC TACGGGGTCTGACGCTCAGTGGAACGAAAACTCACGTTAAGGGA TTTTGGTCATGAGATTATCAAAAAGGATCTTCACCTAGATCCTTTT AAATTAAAAATGAAGTTTTAAATCAAGCCCAATCTGAATAATGTTA CAACCAATTAACCAATTCTGATTAGAAAAACTCATCGAGCATCAA ATGAAACTGCAATTTATTCATATCAGGATTATCAATACCATATTTTT GAAAAAGCCGTTTCTGTAATGAAGGAGAAAACTCACCGAGGCAG TTCCATAGGATGGCAAGATCCTGGTATCGGTCTGCGATTCCGACT CGTCCAACATCAATACAACCTATTAATTTCCCCTCGTCAAAAATAA GGTTATCAAGTGAGAAATCACCATGAGTGACGACTGAATCCGGT GAGAATGGCAAAAGTTTATGCATTTCTTTCCAGACTTGTTCAACAG GCCAGCCATTACGCTCGTCATCAAAATCACTCGCATCAACCAAACC GTTATTCATTCGTGATTGCGCCTGAGCGAGACGAAATACGCGATC GCTGTTAAAAGGACAATTACAAACAGGAATCGAATGCAACCGGC GCAGGAACACTGCCAGCGCATCAACAATATTTTCACCTGAATCAG GATATTCTTCTAATACCTGGAATGCTGTTTTTCCGGGGATCGCAGT GGTGAGTAACCATGCATCATCAGGAGTACGGATAAAATGCTTGAT GGTCGGAAGAGGCATAAATTCCGTCAGCCAGTTTAGTCTGACCAT CTCATCTGTAACATCATTGGCAACGCTACCTTTGCCATGTTTCAGA AACAACTCTGGCGCATCGGGCTTCCCATACAAGCGATAGATTGTC GCACCTGATTGCCCGACATTATCGCGAGCCCATTTATACCCATATA AATCAGCATCCATGTTGGAATTTAATCGCGGCCTCGACGTTTCCCG |
| | | TTGAATATGGCTCATAACACCCCTTGTATTACTGTTTATGTAAGCA GACAGTTTTATTGTTCATGATGATATATTTTTATCTTGTGCAATGTA ACATCAGAGATTTTGAGACACGGGCCAGAGCTGCA |
| 6 | hPGK plasmid | TCGCGCGTTTCGGTGATGACGGTGAAAACCTCTGACACATGCAG CTCCCGGAGACGGTCACAGCTTGTCTGTAAGCGGATGCCGGGAG CAGACAAGCCCGTCAGGGCGCGTCAGCGGGTGTTGGCGGGTGT CGGGGCTGGCTTAACTATGCGGCATCAGAGCAGATTGTACTGAG AGTGCACCATATGCGGTGTGAAATACCGCACAGATGCGTAAGGA GAAAATACCGCATCAGGCGCCATTCGCCATTCAGGCTGCGCAACT GTTGGGAAGGGCGATCGGTGCGGGCCTCTTCGCTATTACGCCAG CTGGCGAAAGGGGGATGTGCTGCAAGGCGATTAAGTTGGGTAA CGCCAGGGTTTTCCCAGTCACGACGTTGTAAAACGACGGCCAGA GAATTCGAGCTCGGTACCTCGCGAATACATCTAGATGTCGACACC GGTGGCCGGCCCAGCCCTGACTGCTATGGACTTTTAGTGCTTGTG CACTGCTACCAGCCATGCCCTGAGTGCACAGGGTTACCCTGGCTG GGGCCTGGAAGCATATAAAAGAAGCTCAGAGTCACATCCAGGTA CACCTTTCACCTGGGCCAGCTTCATTCAGAAGGCCAGGAGGGGG GGATTTTGCCATGTCTGAAGTTCCAGTTATAGGCACACAGTTGCA TCTTACTAGGTCTGGTCTAGGGATCACCAGTGACTTCCAGCCTCT AATTCCACACTGTTCAGCAGGCACCTGTTGTGGCAGGCTGGTCTT AGTTAGCTAGGGCAGGGTTTCTTCACTGGGAGTCCTGGCATTCA GAATGATTAAGGGCTCCCTCTGCCTACCTATCCCCAGTCAACCAG CCCCAGGTGTGTAGCTCTAGTCTGAGACTCATATATCCAGGGTCA CCTCCTGGGATCAAGTGCATGAGCCCAAGCTGGGATCTTTCCCCA GTTACATATTTCTGGACAGTAGGGCTCAGAGTCTCCAGTGCCAGT CCTGTTTCAGAGTATGGAAAAAGTAACCATTGTTACAGCCACAGT CCTTGGGTTAGCCCTAACTCAGCTAATCCAGCAGGCCCAAGGACA CCTGAATATCCAAGGTATGAGTGTGAGGCCCTGTAAGGTAATTCT AGCCCCTTATGCCTAACTTGATTATCAAGACCAAGCTATATCAGG GATGAACCAGAGGCTCCATGACACCCCAGCCACCTAGCTAAACTT GGGGGTTGGGTACAAGTTAGCCCAGAACATACCTTATAGAATTG CCTCTCTAGGGTGAGCAAGGGCCAACCTGCCTATTTGCCCTCTCA CCCCCATTGCAATAGCTTTGGCTCCCAGTACCTCTTCCCTGGCTTC ATTAGCAGATGGCACCCAGCAGATAAAGGTCTATACCCCTGACA AGGGAAACATGGAAAGTATCAGGACCAACATGGTCCACAGCAG AAGTGTCTGGAGTCCATCCTGCATGGCCTTGAGTCCAGGCACAG GAGTCTCCAGTGAGGGTAAACCCCAATCATTGTCCATCCAGGTTT TGCCATAAGACTTGGGCCAGGGTAGCTAAAGCAGATTTACCCCT GCAAGGAGACACCTCATTGGAAACTGAAAGAGACTCCCCACCAG CTTGAAAGGCCAGTCATGCTTTTGCCTGACTCCTGCTCTCTATGCA GTGGCAATCTAAGTGGGAGGTCTGTTCTTCCCAAGAGAGGACCA AGTTTCTGTCCCAAGGCAATAATCCTGTTATCATTGGCTCCTAGCT GCCATTGTTCTGATTGAGGGTTTAAACTCCGGAATTTAAATCCTG CAGGCAGCTGCGCGCTCGCTCGCTCACTGAGGCCGCCCGGGCAA AGCCCGGGCGTCGGGCGACCTTTGGTCGCCCGGCCTCAGTGAGC GAGCGAGCGCGCAGAGAGGGAGTGGCCAACTCCATCACTAGGG GTTCCTTGCAGGCCTCCTAGGCTTGCATGCAGTACTGGGGTTGG |
| | | GGTTGCGCCTTTTCCAAGGCAGCCCTGGGTTTGCGCAGGGACGC GGCTGCTCTGGGCGTGGTTCCGGGAAACGCAGCGGCGCCGACCC TGGGTCTCGCACATTCTTCACGTCCGTTCGCAGCGTCACCCGGAT CTTCGCCGCTACCCTTGTGGGCCCCCCGGCGACGCTTCCTGCTCC GCCCCTAAGTCGGGAAGGTTCCTTGCGGTTCGCGGCGTGCCGGA CGTGACAAACGGAAGCCGCACGTCTCACTAGTACCCTCGCAGAC GGACAGCGCCAGGGAGCAATGGCAGCGCGCCGACCGCGATGGG CTGTGGCCAATAGCGGCTGCTCAGCAGGGCGCGCCGAGAGCAG CGGCCGGGAAGGGGCGGTGCGGGAGGCGGGGTGTGGGGCGGT AGTGTGGGCCCTGTTCCTGCCCGCGCGGTGTTCCGCATTCTGCAA GCCTCCGGAGCGCACGTCGGCAGTCGGCTCCCTCGTTGACCGAA TCACCGACCTCTCTCCCCAGGTGAGTCTATGGGACCCTTGATGTT TTCTTTCCCCTTCTTTTCTATGGTTAAGTTCATGTCATAGGAAGGG GAGAAGTAACAGGGTACACATATTGACCAAATCAGGGTAATTTT GCATTTGTAATTTTAAAAAATGCTTTCTTCTTTTAATATACTTTTTT GTTTATCTTATTTCTAATACTTTCCCTAATCTCTTTCTTTCAGGGCA ATAATGATACAATGTATCATGCCTCTTTGCACCATTCTAAAGAATA ACAGTGATAATTTCTGGGTTAAGGCAATAGCAATATTTCTGCATA TAAATATTTCTGCATATAAATTGTAACTGATGTAAGAGGTTTCATA TTGCTAATAGCAGCTACAATCCAGCTACCATTCTGCTTTTATTTTA TGGTTGGGATAAGGCTGGATTATTCTGAGTCCAAGCTAGGCCCTT TTGCTAATCATGTTCATACCTCTTATCTTCCTCCCACAGGAATTCA GGTACCTGAGCTCTGAGGATCCTTATCAGCCACCATGGAGGAGC TGGTGGTGGAAGTGCGGGGCTCCAATGGCGCTTTCTACAAGGCA TTTGTAAAGGATGTTCATGAAGATTCAATAACAGTTGCATTTGAA AACAACTGGCAGCCTGATAGGCAGATTCCATTTCATGATGTCAGA TTCCCACCTCCTGTAGGTTATAATAAAGATATAAATGAAAGTGAT GAAGTTGAGGTGTATTCCAGAGCAAATGAAAAAGAGCCTTGCTG TTGGTGGTTAGCTAAAGTGAGGATGATAAAGGGTGAGTTTTATG TGATAGAATATGCAGCATGTGATGCAACTTACAATGAAATTGTCA CAATTGAACGTCTAAGATCTGTTAATCCCAACAAACCTGCCACAA AAGATACTTTCCATAAGATCAAGCTGGATGTGCCAGAAGACTTAC GGCAAATGTGTGCCAAAGAGGCGGCACATAAGGATTTTAAAAAG GCAGTTGGTGCCTTTTCTGTAACTTATGATCCAGAAAATTATCAG CTTGTCATTTTGTCCATCAATGAAGTCACCTCAAAGCGAGCACAT ATGCTGATTGACATGCACTTTCGGAGTCTGCGCACTAAGTTGTCT CTGATAATGAGAAATGAAGAAGCTAGTAAGCAGCTGGAGAGTTC AAGGCAGCTTGCCTCGAGATTTCATGAACAGTTTATCGTAAGAGA AGATCTGATGGGTCTAGCTATTGGTACTCATGGTGCTAATATTCA GCAAGCTAGAAAAGTACCTGGGGTCACTGCTATTGATCTAGATG AAGATACCTGCACATTTCATATTTATGGAGAGGATCAGGATGCA GTGAAAAAAGCTAGAAGCTTTCTCGAATTTGCTGAAGATGTAATA CAAGTTCCAAGGAACTTAGTAGGCAAAGTAATAGGAAAAAATGG AAAGCTGATTCAGGAGATTGTGGACAAGTCAGGAGTTGTGAGG GTGAGGATTGAGGCTGAAAATGAGAAAAATGTTCCACAAGAAG AGGAAATTATGCCACCAAATTCCCTTCCTTCCAATAATTCAAGGG TTGGACCTAATGCCCCAGAAGAAAAAAAACATTTAGATATAAAG |
| | | GAAAACAGCACCCATTTTTCTCAACCTAACAGTACAAAAGTCCAG AGGGTGTTAGTGGCTTCATCAGTTGTAGCAGGGGAATCCCAGAA ACCTGAACTCAAGGCTTGGCAGGGTATGGTACCATTTGTTTTTGT GGGAACAAAGGACAGCATCGCTAATGCCACTGTTCTTTTGGATTA TCACCTGAACTATTTAAAGGAAGTAGACCAGTTGCGTTTGGAGA GATTACAAATTGATGAGCAGTTGCGACAGATTGGAGCTAGTTCT AGACCACCACCAAATCGTACAGATAAGGAAAAAAGCTATGTGAC TGATGATGGTCAAGGAATGGGTCGAGGTAGTAGACCTTACAGAA ATAGGGGGCACGGCAGACGCGGTCCTGGATATACTTCAGGAACT AATTCTGAAGCATCAAATGCTTCTGAAACAGAATCTGACCACAGA GACGAACTCAGTGATTGGTCATTAGCTCCAACAGAGGAAGAGAG GGAGAGCTTCCTGCGCAGAGGAGACGGACGGCGGCGTGGAGG GGGAGGAAGAGGACAAGGAGGAAGAGGACGTGGAGGAGGCTT CAAAGGAAACGACGATCACTCCCGAACAGATAATCGTCCACGTA ATCCAAGAGAGGCTAAAGGAAGAACAACAGATGGATCCCTTCAG ATCAGAGTTGACTGCAATAATGAAAGGAGTGTCCACACTAAAAC ATTACAGAATACCTCCAGTGAAGGTAGTCGGCTGCGCACGGGTA AAGATCGTAACCAGAAGAAAGAGAAGCCAGACAGCGTGGATGG TCAGCAACCACTCGTGAATGGAGTACCCTAATGACACATTGTGTG ATATCTCTAGGATGCTCGCTTTCTTGCTGTCCAATTTCTATTAAAG GTTCCTTTGTTCCCTAAGTCCAACTACTAAACTGGGGGATATTAT GAAGGGCCTTGAGCATCTGGATTCTGCCTAATAAAAAACATTTAT TTTCATTGCAATGATGTATTTAAATTATTTCTGAATATTTTACTAAA AAGGGAATGTGGGAGGTCAGTGCATTTAAAACATAAAGAAATG AAGAGCTAGTTCAAACCTTGGGAAAATACACTATATCTTAAACTC CATGAAAGAAGGTGAGGCTGCAAACAGCTAATGCACATTGGCAA CAGCCCCTGATGCCTATGCCTTATTCATCCCTCAGAAAAGGATTC AAGTAGAGGCTTGATTTGGAGGTTAAAGTTTTGCTATGCTGTATT TTAAGATCTGTGTTAAGATACATTGATGAGTTTGGACAAACCACA ACTAGAATGCAGTGAAAAAAATGCTTTATTTGTGAAATTTGTGAT GCTATTGCTTTATTTGTAACCATTATAAGCTGCAATAAACAAGTTA TGCATGGACATGTTTGATCATGGTTGTAAGTATCAAGGTTACAAG ACAGGTTTAAGGAGACCAATAGAAACTGGGCTTGTCGAGACAGA GAAGACTCTTGCGTTTCTGATAGGCACCTATTGGTCTTACTGACA TCCACTTTGCCTTTCTCTCCACAGAGATCTGTGTGTTGGTTTTTTCT TAAGGTGTGATTAATGAGCTACCAGGTCTCGAGGAGGAACCCCT AGTGATGGAGTTGGCCACTCCCTCTCTGCGCGCTCGCTCGCTCAC TGAGGCCGGGCGACCAAAGGTCGCCCGACGCCCGGGCTTTGCCC GGGCGGCCTCAGTGAGCGAGCGAGCGCGCAGCTGCCTGCAGGT TATAACCTGCAGGTTAATTAAGCCTTGTAGTCTAGCCAGGGTGTA TAACCCCTCCAGCCCATGTTCAAAGAGCTGTCTTGCCTAGCCAGT TGCACATACAAAGTGATAAATGGGAGCTGGCATTGTGCCCTTGG AGGCACTCTGAAGGATCACCAGTGAACCCAGCAGCAAAGATACA TAGGCTCTTAATTCATGCCAGGATCTCAGAGAGGCAATGGCTTGC ACAATCAGGGGTTATTCTGACAGCATGAACTCTGGTGCCAGACA ATTTTATGTATCAGGCAATGTGCATTACTTGAGGTGGATTACAGA CCCAGTGAGTAACCCAGGACCAGGAGTAAACAGGCCCTAATCCC |
| | | AGCTTGATTGACACCAGGCTTGAAGATCCTTACCATTATGAAAAC AGCACATGGTCTGTTGACAATTACTTGTAGCATAGGTAGCCCAGG CAGAGTGGCAGACAGGGTACTGATAGTAGTTAGAGAACCTCCCA GATAAGCTACCTGACCTTCTCTAATCTTGAGTTCTGTGGGCAACC AGCCCAGTGAGCATCTTGGGTTCTTGGAAATCCAGACCCTACTCA CCTGAGTATAAATGGGGCAGCCACCCACCCAGACTGATCCATCCT ACCTTTGAGGCTACCCATGAGGTAATTAGGCCAGAATGTGAAAG GGAGGAGCCCAGAGCATTGTTCCTTGTATTACCATTGGGAACCTT GTTAGATGGGGAGGAATGCACTTCAGCCACCCTAGAGGAGTTGA GTCATTAGAAGAAGAAGGCTGCTTCCCCATCAGGAGAAGATCCA GCCAGTAAGTATATAGGTTACAACTGCCAGGTACTATGGGCTTCT CCAGACCCTTCCTACCCAGGAACTAGAAGGTTGGAGCCTAAAGT CCCCTACCCATGTGCTGACTGATCCAGAGTTACACTCCCTCAGACT CATCCTCCAGACAGGGTTCCCAGTTATTAGGATTGCAATAACCAT CCAGTTCCCAAACTATTCCAGCTTCCTATCCAGTAATAAGCCCTTA TTCTTTAACCTCTGAAGAAACCCTGAGTGAACAGGCTGTGCAGG GCTCCTGTCACTTCTGTCAGCCCAGATAGGTATGAAATCTCTTCAA ATGTATTGCCAATGACATTGCCAGGCACAGATTCTCCCAGTTACC CAACCAGGAACACCAGCTAGTGTCAAACAGTCAATGCCCTTCTAC CAGCCATTTGAGACACTACCAGGCAGGCAAAGCCAAGTGGCTTC ACTCCTTATTTATATTAGCTCAGAGGAGCCATCTAACAGCTCTTAC TCAACTAGACTAGCTGCATGAACAGTGTACAGCTAGCTGCGCAAT CGGATCCCGGGCCCGTCGACTGCAGAGGCCTGCATGCAAGCTTG GTGTAATCATGGTCATAGCTGTTTCCTGTGTGAAATTGTTATCCG CTCACAATTCCACACAACATACGAGCCGGAAGCATAAAGTGTAA AGCCTGGGGTGCCTAATGAGTGAGCTAACTCACATTAATTGCGTT GCGCTCACTGCCCGCTTTCCAGTCGGGAAACCTGTCGTGCCAGCT GCATTAATGAATCGGCCAACGCGCGGGGAGAGGCGGTTTGCGT ATTGGGCGCTCTTCCGCTTCCTCGCTCACTGACTCGCTGCGCTCG GTCGTTCGGCTGCGGCGAGCGGTATCAGCTCACTCAAAGGCGGT AATACGGTTATCCACAGAATCAGGGGATAACGCAGGAAAGAACA TGTGAGCAAAAGGCCAGCAAAAGGCCAGGAACCGTAAAAAGGC CGCGTTGCTGGCGTTTTTCCATAGGCTCCGCCCCCCTGACGAGCA TCACAAAAATCGACGCTCAAGTCAGAGGTGGCGAAACCCGACAG GACTATAAAGATACCAGGCGTTTCCCCCTGGAAGCTCCCTCGTGC GCTCTCCTGTTCCGACCCTGCCGCTTACCGGATACCTGTCCGCCTT TCTCCCTTCGGGAAGCGTGGCGCTTTCTCATAGCTCACGCTGTAG GTATCTCAGTTCGGTGTAGGTCGTTCGCTCCAAGCTGGGCTGTGT GCACGAACCCCCCGTTCAGCCCGACCGCTGCGCCTTATCCGGTAA CTATCGTCTTGAGTCCAACCCGGTAAGACACGACTTATCGCCACT GGCAGCAGCCACTGGTAACAGGATTAGCAGAGCGAGGTATGTA GGCGGTGCTACAGAGTTCTTGAAGTGGTGGCCTAACTACGGCTA CACTAGAAGAACAGTATTTGGTATCTGCGCTCTGCTGAAGCCAGT TACCTTCGGAAAAAGAGTTGGTAGCTCTTGATCCGGCAAACAAA CCACCGCTGGTAGCGGTGGTTTTTTGTTTGCAAGCAGCAGATTA CGCGCAGAAAAAAAGGATCTCAAGAAGATCCTTTGATCTTTTCTA CGGGGTCTGACGCTCAGTGGAACGAAAACTCACGTTAAGGGATT |
| | | TTGGTCATGAGATTATCAAAAAGGATCTTCACCTAGATCCTTTTAA ATTAAAAATGAAGTTTTAAATCAAGCCCAATCTGAATAATGTTAC AACCAATTAACCAATTCTGATTAGAAAAACTCATCGAGCATCAAA TGAAACTGCAATTTATTCATATCAGGATTATCAATACCATATTTTT GAAAAAGCCGTTTCTGTAATGAAGGAGAAAACTCACCGAGGCAG TTCCATAGGATGGCAAGATCCTGGTATCGGTCTGCGATTCCGACT CGTCCAACATCAATACAACCTATTAATTTCCCCTCGTCAAAAATAA GGTTATCAAGTGAGAAATCACCATGAGTGACGACTGAATCCGGT GAGAATGGCAAAAGTTTATGCATTTCTTTCCAGACTTGTTCAACA GGCCAGCCATTACGCTCGTCATCAAAATCACTCGCATCAACCAAA CCGTTATTCATTCGTGATTGCGCCTGAGCGAGACGAAATACGCGA TCGCTGTTAAAAGGACAATTACAAACAGGAATCGAATGCAACCG GCGCAGGAACACTGCCAGCGCATCAACAATATTTTCACCTGAATC AGGATATTCTTCTAATACCTGGAATGCTGTTTTTCCGGGGATCGC AGTGGTGAGTAACCATGCATCATCAGGAGTACGGATAAAATGCT TGATGGTCGGAAGAGGCATAAATTCCGTCAGCCAGTTTAGTCTG ACCATCTCATCTGTAACATCATTGGCAACGCTACCTTTGCCATGTT TCAGAAACAACTCTGGCGCATCGGGCTTCCCATACAAGCGATAG ATTGTCGCACCTGATTGCCCGACATTATCGCGAGCCCATTTATACC CATATAAATCAGCATCCATGTTGGAATTTAATCGCGGCCTCGACG TTTCCCGTTGAATATGGCTCATAACACCCCTTGTATTACTGTTTAT GTAAGCAGACAGTTTTATTGTTCATGATGATATATTTTTATCTTGT GCAATGTAACATCAGAGATTTTGAGACACGGGCCAGAGCTGCA |

### Example 2. Optimization of Dosing, Timing and Delivery Route for FMRP-Expressing AAV Vectors

To determine the best delivery routes for FMRP-expressing AAV vectors, and well as dosing and timing optimized for CNS-specific expression of FMRP, in vivo studies using the scAAV9-CB-FMR1 and AAV-CAG-FMR1 were conducted in a mouse model.

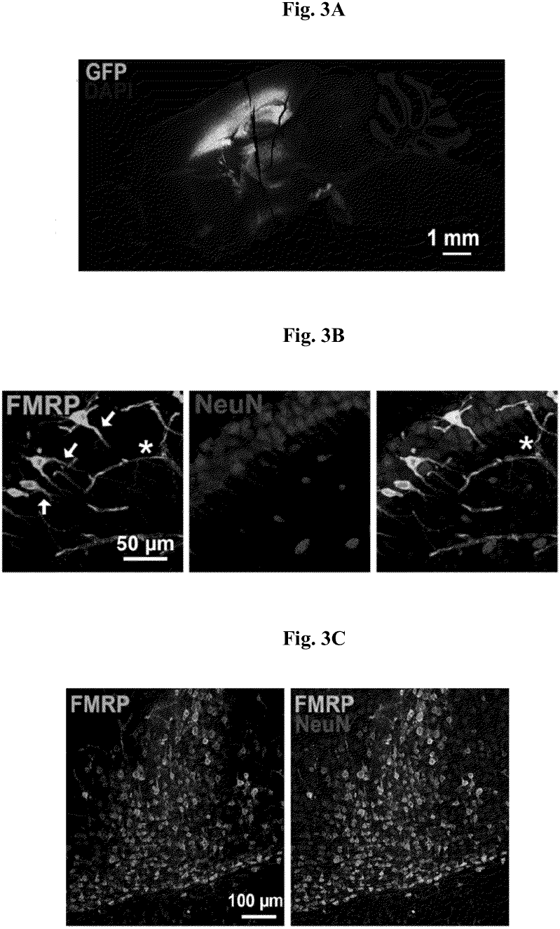

To assess timing of transduction and recombinant gene expression using scAAV, ~20 million vg scAAV-GFP were intracerebroventricularly (ICV) injected into 6-week old wildtype mice. ICV delivery minimizes systemic immune responses and side effects, while guaranteeing wide spread administration within the brain. Two weeks after administration of the viral injections, mice were transcardially perfused (4% paraformaldehyde) and brains were postfixed overnight, cryoprotected in 30% sucrose, and flash-frozen. Brain sections were mounted on microscope slides for processing and were imaged using a confocal microscope. FIG. 3A shows scAAV-GFP expression in the cortical and hippocampus region of the scAAV-GFP-injected wild-type mouse brain.

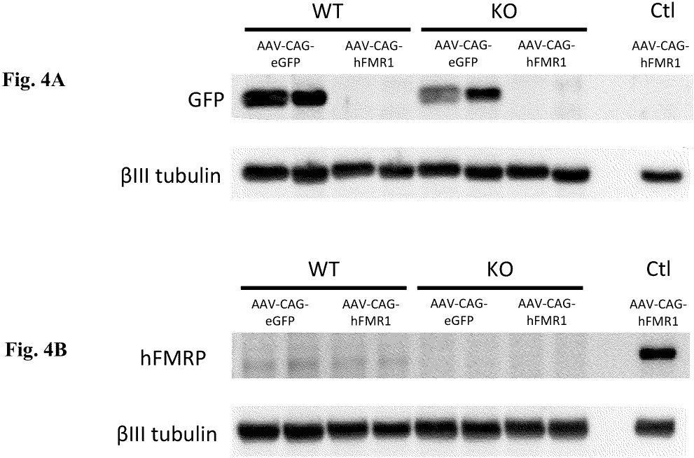

Additionally, ~20 million vg AAV-CAG-FMR1 were ICV injected into 6-week old wild-type mice. Two weeks after viral injections, mice were transcardially perfused (4% paraformaldehyde) and brains were postfixed overnight, cryoprotected in 30% sucrose, and flash-frozen. Brain sections were mounted on microscope slides for processing and fluorescent immunostainings were performed similar to the methods described in Gross et al., Cell Rep. 2015;11(5):681-688, the disclosure of which is incorporated herein in its entirety. FIGS. 3B and 3C show cortical and hippocampal neurons (marked with the immunofluorescent marker NeuN) with increased FMRP protein expression after two weeks. Total protein expression of AAV-CAG-FMR1 and AAV-CAG-GFP was also assessed in brain slices containing cortex, hippocampus, midbrain, and cerebellum harvested from ICV injected mice. Briefly, ~40 million vg of AAV-CAG-FMR1 or AAV-CAG-GFP were ICV injected into 6-7 week old wild-type mice and 6-7 week old Fmr1 knockout (KO) mice. Ten weeks after viral injections, brains were harvested and brain slides were collected and processed for western blot analysis. FIGS. 4 shows that GFP was clearly detectable by western blot in cortex and hippocampus, whereas FMRP was below the detection limit in mice injected with regular AAV containing FMRP or GFP under the CAG promoter. Western blotting and immunohistochemistry analyses of the brain sections also assess cell death and gliosis in the injected mice, aiding in the identification of a dose that leads to moderate FMRP expression (70-110%) with no signs of cell death or gliosis.

### Example 3: Optimization of Dosing, Timing and Delivery Route for FMRP-Expressing AAV Vectors

To further optimize the dosing, timing, and delivery route for FMRP-expressing AAV vectors, said vectors are administered to Fmr1 knockout (KO) mice, and functional and physiological outcomes are assessed.

Mice are administered vector(s), for example, CAGWPRE, CAGdelWPRE, or hPGK, at P21 via intravenous (IV) or combined (IV+ICV) administration routes. Control groups comprise WT and KO mice receiving vehicle via combo administration. Mice in the experimental group receive either a low dose (e.g., 1E13-5E13vg/kg) or a high dose (e.g.,8E13-2E14vg/kg) of the administered vector. Mice in all groups undergo behavioral testing 60 days post dose. Behavioral testing includes assessment of nesting behavior, evaluation of performance in the Morris Water Maze Task, and functional neurophysiological assessments using electroencephalography (EEG). Mice in all groups are subjected to terminal assessments of biodistribution. Each experimental and control group consists of approximately 10 mice.

In another study, mice are administered vector(s), for example, CAGWPRE, CAGdelWPRE, or hPGK, at P21 (“pediatric”) or P42 (“older”) via intravenous (IV) or combined (IV+ICV) administration routes. Control groups comprise WT and KO mice receiving vehicle via IV or IV+ICV administration at either P21 or P42. Mice in the experimental groups receive one of a range of doses (e.g., 1E13-2E14 vg/kg) of the administered vector. Mice in all groups undergo behavioral testing 90 days post dose. Behavioral testing includes assessment of nesting behavior, evaluation of performance in the Morris Water Maze Task, and functional neurophysiological assessments using electroencephalography (EEG). Mice in all groups are subjected to terminal assessments of biodistribution. Each experimental and control group consists of approximately 10 mice.

The results of the above experiments are analyzed to determine which dosing regimen, timing, and administration route provide superior delivery of transgene to all parts of the brain and body, as well as superior rescue of functional and behavioral deficits in Fmr1 KO mice. Given that FMR1 gene is ubiquitously expressed in tissues, it is anticipated that the broad distribution of corrective transgene achievable by one or more of the tested administration conditions will be beneficial to the treatment of Fragile X Syndrome.

### Example 4. Behavioral Analyses in a Mouse Model of FXS Following Administration of FMRP-Expressing AAV Vectors

Fmr1 knockout (KO) mice do not express FMRP and replicate the human phenotypes associated with FXS including brain hyperexcitability and behavioral and cognitive deficits. This suggested that Fmr1 KO mice are not only an excellent model for FXS but that behavioral paradigms testing prefrontal cortical function in Fmr1 KO mice could be used to assess the potential of therapeutic strategies to rescue cognitive impairment in FXS by AAV gene therapy.

Fmr1 KO mice were generated in a similar manner as described in Gross et al., Cell Rep. 2015;11(5):681-688, the disclosure of which is incorporated herein in its entirety. In brief, Fmr1 KO mice were generated by crossing female Fmr1HET mice with male Pik3cb heterozygous mice and were genotyped by PCR. Knockout mouse lines were backcrossed into C57BL/6J background at least four times (Pik3cbHET) or more than ten times (Fmr1HET).

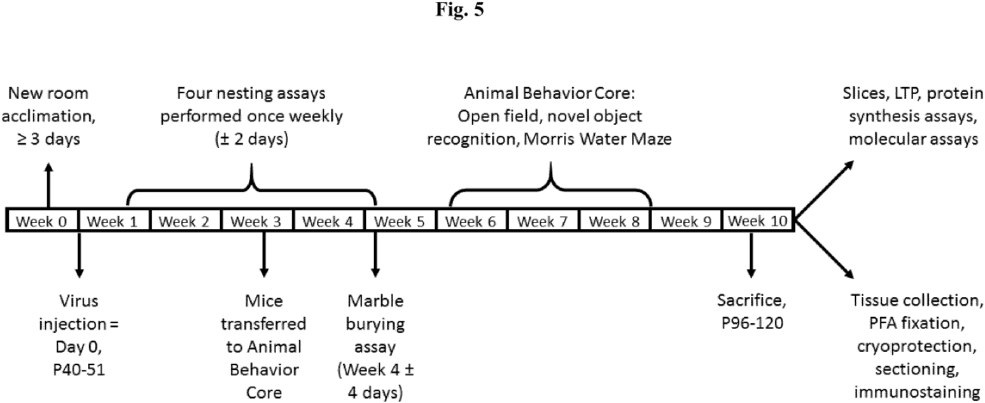

Fmr1 KO mice and wild-type control mice were subjected to behavioral and functional assessments following AAV administration according to the timeline shown in FIG. 5. In brief, Fmr1 KO mice and wild type mice were ICV injected with 40-60 million viral genomes per mouse of either AAV-CAG-FMR1 or AAV-CAG-GFP. Mice were between 6-7 weeks of age at the time of injection. Mice were kept alive for about 10 weeks after virus injection and were subjected to multiple behavioral assays during this time (nesting, marble burying, open field activity, novel object recognition and Morris water maze).

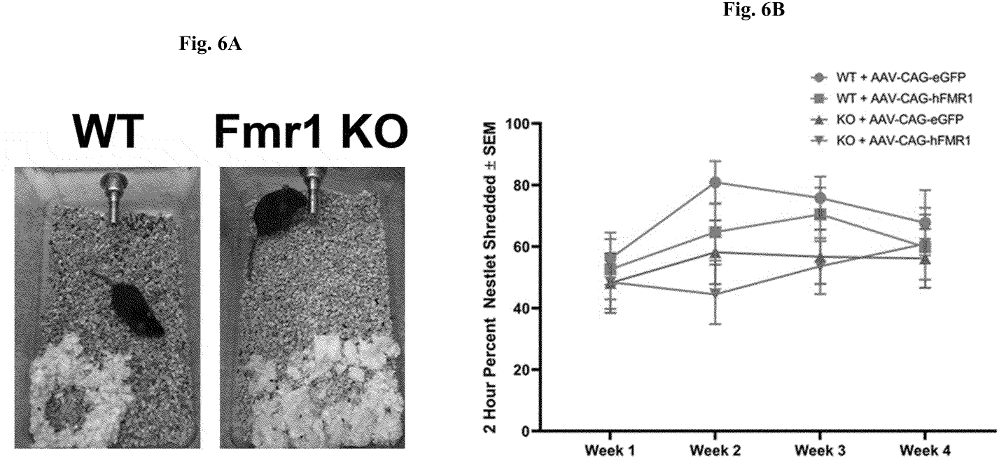

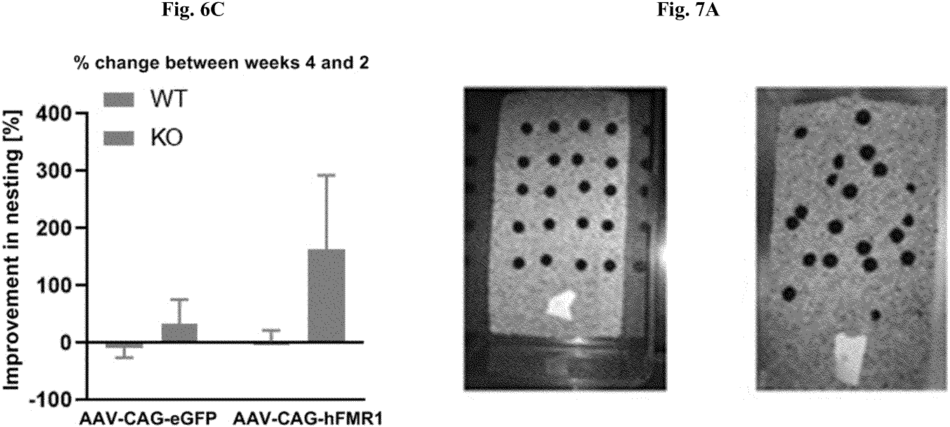

Nest building was assessed weekly for 4 weeks starting one week after virus injection. Briefly, mice were placed in a fresh cage with standard bedding supplemented with 3 grams of fresh nestlet at the start of the experiment. Nests were assessed 2 hours later using a scoring system as described in Gross et al., Cell Rep. 2015;11(5):681-688, the disclosure of which is incorporated herein in its entirety. FIG. 6A shows an example of a wild-type mouse and a Fmr1 KO mouse shredding nestlet materials 2 hours after receiving a fresh nestlet. More shredding is indicative of “home cage behavior,” which translates to “social behavior” in humans. FIG. 6B shows that overall, Fmr1 KO mice shredded less nestlet. Fmr1 KO mice injected with AAV-CAG-FMR1 shredded increasing amounts of nestlet between 2 and 4 weeks, whereas the AAV-CAG-GFP injected mice did not improve, suggesting positive effects of FMRP expression (FIG. 6C). These tests also showed that all mice engaged in nest building, confirming that their overall health was not affected by the viral vectors.

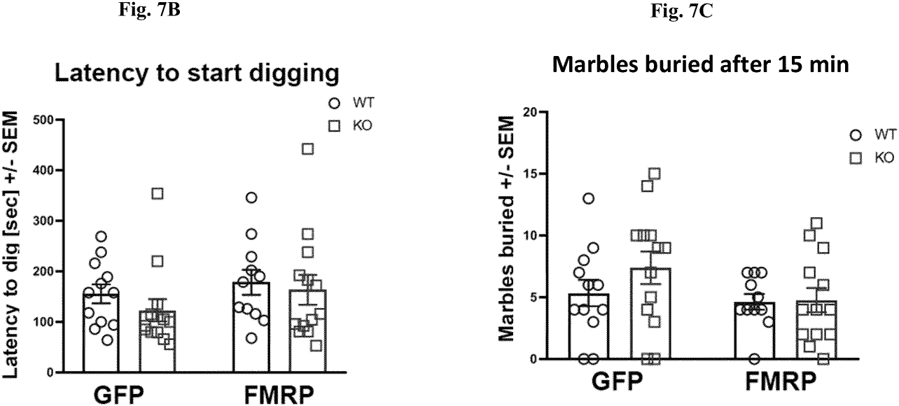

Excessive marble burying is suggestive of perseverative or anxiety-related behavior in mice and was altered in Fmr1 KO mice. Four weeks after AAV administration, the Fmr1 KO and wild-type mice were subjected to marble bury assays. Briefly, mice were placed in cages with twenty blue small glass beads arranged in a 5 × 4 grid on fresh bedding (ca. 8 cm deep). After 15 minutes, mice were removed and marbles covered 50% or more were scored as “buried.” Latency to start digging to bury marbles was also measured during the 15 minutes. Mice were tested between 12 PM and 3 PM, and were tested in nesting behavior prior to marble burying. FIG. 7A shows an example of marble burying behavior in mice. The left panel shows marble arrangement before mice were put in and the right panel shows marble positions after the mice were put it. GFP-injected Fmr1 KO mice (representing Fmr1 KO mice as GFP has no impact on Fmr1 KO mice) on average had a reduced latency to start burying and buried more marbles than wild type mice; injection of the FMRP-expressing AAV vector rescued the reduced latency (FIGS. 7B and 7C).

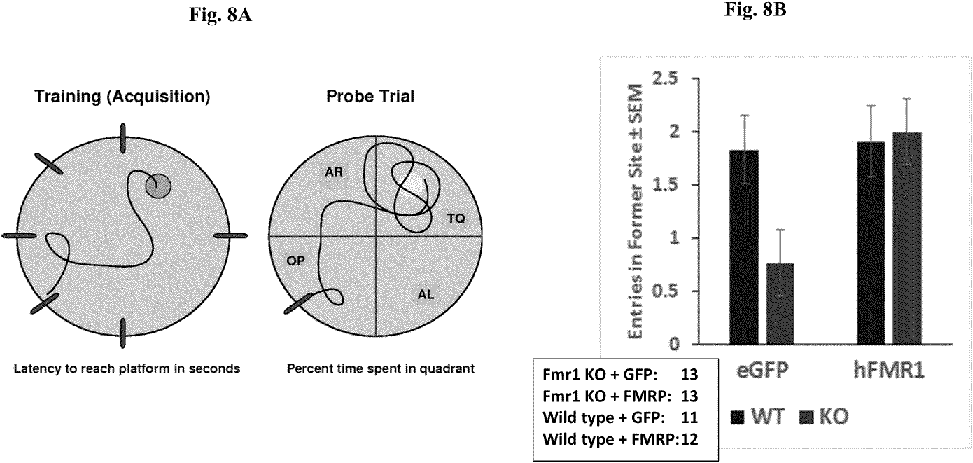

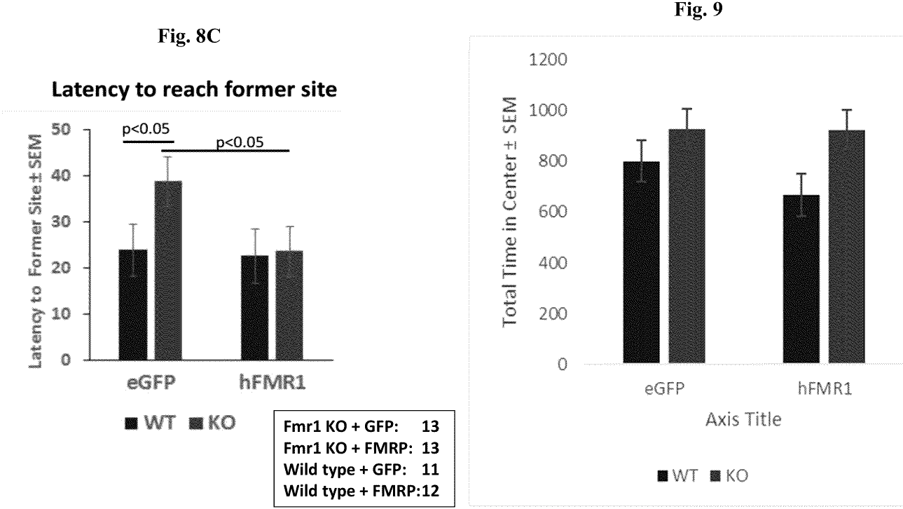

AAV-CAG-FMR1 or AAV-CAG-GFP injected Fmr1 KO and wild type mice were subjected to Morris Water Maze assays six-eight weeks after AAV injection. The Morris Water Maze is generally used to determine to what extent the hippocampus plays a role in spatial learning. FIG. 8A shows a diagram of the Morris Water Maze assays that were performed as disclosed herein. During the training (acquisition) trial of the Morris water maze, the mouse was placed in the water, facing the wall, at one of the six starting points, indicated by the brown marks in FIG. 8A. The mouse was allowed to swim for up to 60 seconds or until it found the platform. The time to reach the platform (latency) was measured in seconds. In the probe trial, the mouse was placed in the pool in the quadrant opposite (OP) of the platform, which had been removed. In the probe trial, the time spent in each quadrant and platform crosses was measured. Quadrant TQ was the target quadrant, the area of the pool in which the platform was located. OP was the opposite quadrant of the TQ. AR and AL were the adjacent right and left quadrants of the target quadrant when one was looking down on the pool. All groups acquired the task at a similar rate and were able to find the hidden platform at the end of training. In the reversal task, when the location of the hidden platform was moved to the opposite quadrant of the maze, GFP-injected Fmr1 KO mice had less entries into the former quadrant (FIG. 8B) and the latency to the former platform location was increased (FIG. 8C), suggesting that GFP-injected Fmr1 KO mice remembered the location of the platform less accurately, which is in line with a memory deficit. FMRP10 injected Fmr1 KO mice were indistinguishable from wild type mice (FIGS. 8B and 8C), suggesting this memory deficit was improved.

AAV-CAG-FMR1 or AAV-CAG-GFP injected Fmr1 KO and wild type mice were also subjected to open field activity assays six-eight weeks after AAV injection. Open field activity assays measure hyperactivity and/or anxiety. Briefly, mice were habituated to the experimental room for 30 minutes before the start of the test. Mice were placed into the center of a clear Plexiglas (40 × 40 × 30) cm open field arena and allowed to explore for 15 minutes. Illumination was provided by overhead lights (~800 lux) inside the arenas and experiments were done in the presence of white noise at 55 decibels (dB). Data were collected at 2 minute intervals controlled by a Digiscan optical animal activity system. Data were pooled for computer-designated peripheral and central sectors and expressed as an average per genotype. These studies showed that GFP-injected Fmr1 KO mice spent more time in the center of an open field arena (2-way ANOVA, effect of genotype p=0.02); however, no differences between GFP-injected wild type and Fmr1 KO mice were observed (FIG. 9). Overall, virally expressed hFMRP does not drastically affect altered open field activity.

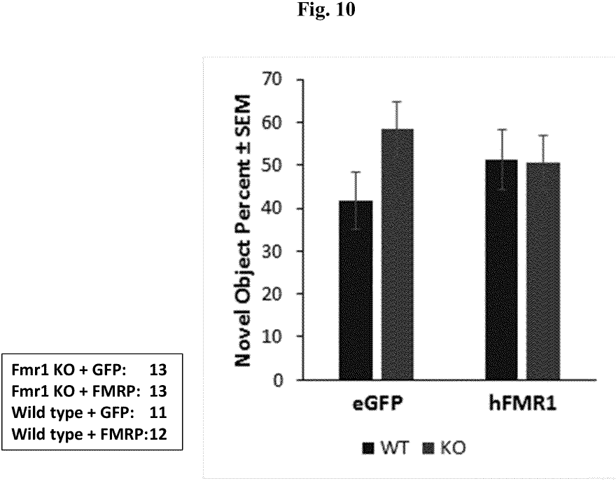

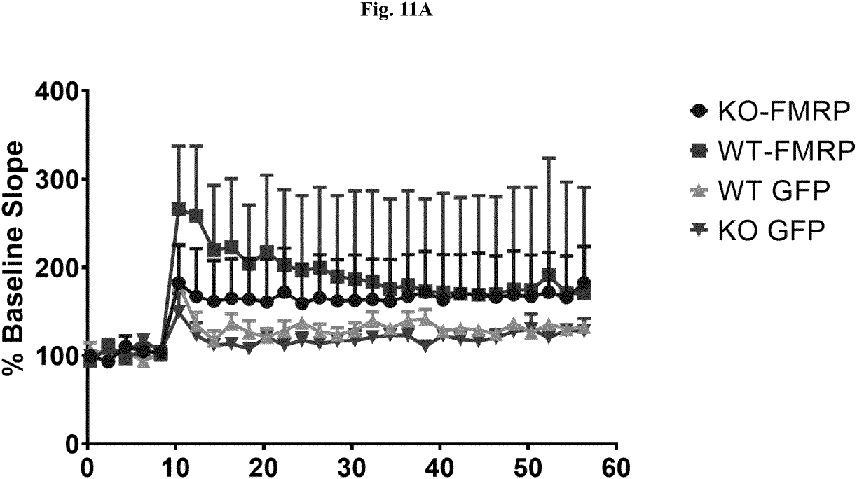

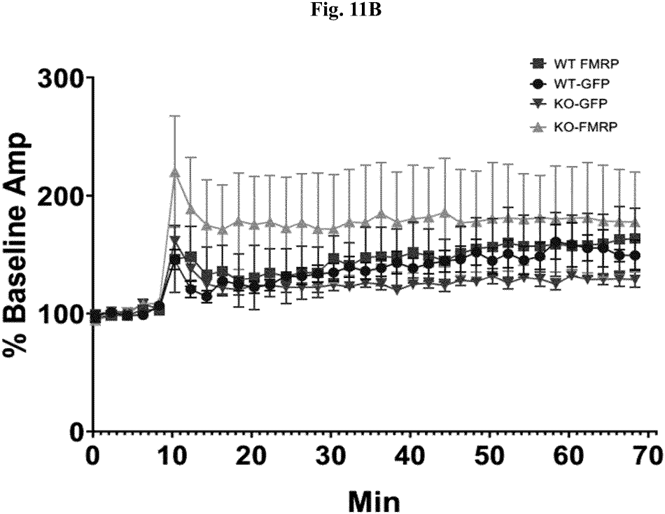

AAV-CAG-FMR1- or AAV-CAG-GFP-injected Fmr1 KO and wild type mice were subjected to novel object recognition assays six-eight weeks after AAV injection. The novel object recognition assay relies on the innate preference of mice to explore a novel versus a familiar object, which was speculated to be impaired in Fmr1 KO mice. Here, inanimate, wooden, and neutral colored objects were used in the novel object recognition tests disclosed in this Example. Objects were first tested for neutral preference strength using a naive cohort of separate wild-type mice, with objects that elicited either a strong attraction or an aversive response being discarded. On day one, mice were habituated to a round, white arena (30 cm diameter) for 30 minutes. The following day, mice were exposed to the arena with several equally spaced objects within it for 15 minutes. Interaction time with each object was calculated for each mouse and the two objects that evoked median responses were used as ‘familiar’ objects for the next two days of testing. On days three and four, mice were presented with familiar objects within specific areas (counter-balanced locations for presentation of objects) of the arena for 15 minutes. On day five, one of the ‘familiar’ objects was replaced with a fourth, ‘novel’ object and interaction behavior of the mice was tested for 15 minutes. The entire 15 minute interaction times were recorded where the mice were exposed to four objects (three familiar and one novel). Interaction parameters were defined as contact with the object (tail only excluded) or facing the object (distance <2 cm). The preference index (PI) was calculated by the time spent interacting with the novel object divided by the amount of time exploring both the novel and familiar objects. All experiments were recorded and then scored by two observers blind to the genotypes and treatment groups. As shown in FIG. 10, all mice showed a preference for the novel object, and there were no significant differences between groups.

Overall, most of the behavioral assays that were performed in this Example showed differences between GFP-injected Fmr1 KO mice and wild type mice which was indicative of the behavioral phenotype of FXS. FMR1-injected Fmr1 KO mice demonstrated behaviors more similar to wild-type mice, indicating that, surprisingly, even small amounts of FMRP reintroduced into cortex and hippocampus of adult mice improved behavior. The results suggested that virally expressed FMRP has the potential to improve at least home cage/social behavior (nesting), anxiety-related and perseverative behaviors (marble burying) and learning and memory (Morris water maze).

### Example 5. Functional Analyses in Brain Slices Harvested From a Mouse Model of FXS Following Administration of FMRP-Expressing AAV Vectors

1mrl KO mice and wild type mice were ICV injected with 40-60 million viral genomes per mouse of either AAV-CAG-FMR1 or AAV-CAG-GFP. Mice were between 6-7 weeks of age at the time of injection. As reflected in the timeline shown in FIG. 5, mice were kept alive for about 10 weeks after virus injection and were subjected to multiple behavioral assays during this time. After at least 5 days after the last behavioral assays (~10 weeks after surgery), brain tissue was collected from all mice and used for functional assays in slices (e.g., using multielectrode array (MEA)) to measure long-term potentiation (LTP) and protein synthesis assays) as well as expression analyses (immunohistochemistry and western blotting).

### (I) Long-Term Potentiation (LTP)

Long-term potentiation, an enduring form of enhancement of synaptic connections following a stimulus, is a cellular correlate of learning and memory. Briefly, transverse hippocampal slices (300 µm) through the mid-septotemporal hippocampus were prepared with a vibratome in ice-cold artificial CSF (ACSF) (in mm: 124 NaCl, 3 KCl, 1.25 KH2PO4, 3.4 CaCl2, 2.5 MgSO4, 26 NaHCO3, and 10 dextrose, pH 7.35). Slices from both genotypes and treatment groups were run simultaneously. Slices were maintained at 31 ± 1° C. in an interface recording chamber with the slice surface exposed to warm, humidified 95% O2/5% CO2 and continuous ACSF perfused at a rate of 60-70 ml/h. Slices equilibrated to the chamber for at least 1 hour before recordings were initiated. After incubation, one slice was selected and positioned on the MED64 probe in such a way that the whole HF was entirely covered by the 8 × 8 array. Once the slice settled, a netting ballast (U-shaped platinum wire with regularly spaced hair pieces) was carefully disposed on the slice to immobilize it. For the electrophysiological recordings, the probes with immobilized slices were connected to the stimulation/recording component of MED64. The slice was continuously perfused with oxygenated, fresh ACSF at the rate of 2-3 ml/min with the aid of a peristaltic pump. After a 20 minute recovery of the slice, one of the 64 available planar microelectrodes was selected from the 64-switch box for stimulation following visual observation through a charge coupled device camera connected to an inverted microscope. When not specified, monopolar, biphasic constant current pulses (30-199 µA, 0.1 ms duration) generated by the data acquisition software were applied to the PP at 0.1 Hz. Field potentials evoked at the remaining sites were amplified by the 64-channel main amplifier and then digitized at a 20 kHz sampling rate. The digitized data were displayed on the monitor screen and stored on the hard disk of a microcomputer.

Five successive responses were averaged automatically in real time by the recording system. The viability of the slices was kept constant across different sets of recording sessions by measuring the threshold for evoking fEPSP of adequate amplitude. For LTP induction, the TBS protocol was used, which consisted of 10 bursts, each containing 4 pulses at 100 Hz with an inter-burst interval of 200 ms. It is widely accepted that such a protocol resembles in vivo conditions and has been suggested as a method to establish a link between artificial and natural synaptic activity. In addition, LTP induced by such stimulation appears to be more robust and stable than that induced by other means. To standardize tetanization strength in different experiments, the TBS strength was set at an intensity evoking almost half of the maximal magnitude of fEPSP. After TBS, the test stimulus was repeatedly delivered (at the identical intensity as baseline) once every 10 minutes for more than 2 hours to allow for the observation of any changes in LTP magnitude and duration.

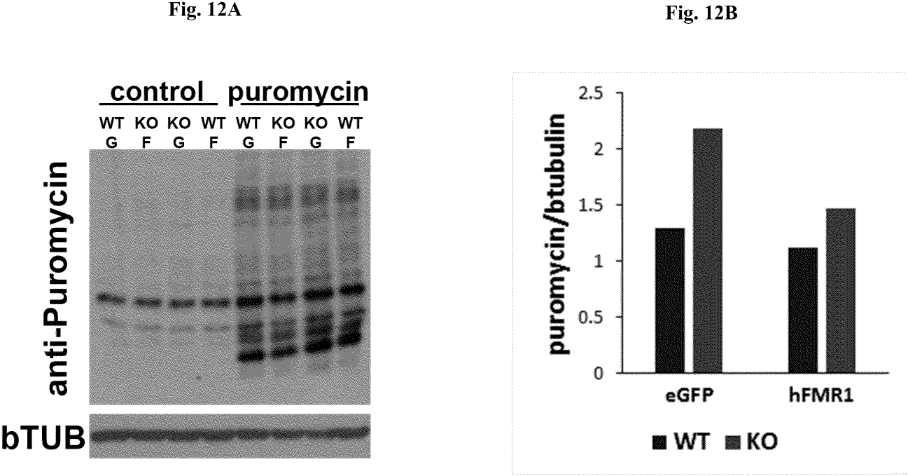

TBS-LTP was shown to be impaired in Fmr1 KO hippocampus. Here, LTP was recorded from f5 Fmr1 KO mice injected with FMRP-expressing AAV, 7 Fmr1 KO mice with GFP-expressing AAV, 6 wild type mice injected with FMRP-expressing AAV, and 5 wild type mice with GFP-expressing AAV. Data analyses with 2-3 mice in each group suggested a slight deficit in GFP-injected Fmr1 KO slices compared to GFP-injected wild type slices, as reported, and an overall increase of LTP in both genotypes after FMRP injection (FIG. 11A). The assay was repeated under the same conditions except measurements were collected for 70 minutes to assess late phase of LTP. FIG. 11B shows that the late phase of LTP (min 30-70, purple triangles) was impaired in GFP-injected Fmr1 KO mice. Additionally, FMRP injection enhanced LTP in the Fmr1 KO mice, but not in the FMRP injected wild type mice (FIG. 11B). These functional analyses support the data disclosed in Example 3 which showed improvement in the hippocampus-dependent Morris Water Maze learning assay (FIGS. 8B-8C).

### (II) Protein Synthesis

Long-term synaptic plasticity, such as learning and memory, depend on the neurons’ capability to synthesize new proteins in response to a stimulus. Protein synthesis rates in FXS mouse models and cells from patients with FXS have been shown to be increased and stimulus-insensitive, i.e. not enhanced after a plasticity-inducing stimulus. In addition, enhanced and dysregulated protein synthesis rates are a pivotal characteristic of FXS (and general autism) and believed to underlie deficits in behavior and cognition. Accordingly, a treatment strategy for FXS can be “therapeutic” if it rescues protein synthesis defects in FXS. To assess protein synthesis rates in wild-type and Fmr1 KO mice injected with either GFP- or FMRP-expressing AAV, the cortical and hippocampal slices prepared for LTP electrophysiology were then used for protein synthesis assays using puromycin incorporation into nascent peptide chains followed by western blot analysis, a method that consistently showed increased protein synthesis rates in Fmr1 KO brains. Puromycinylation assays were performed in 2 Fmr1 KO mice injected with FMRP-expressing AAV, 5 Fmr1 KO mice with GFP-expressing AAV, 5 wild type mice injected with FMRP-expressing AAV, and 4 wild type mice with GFP-expressing AAV. FIGS. 12A and 12B show cortical slices with increased protein synthesis 5 rates in the GFP-injected Fmr1 KO slice compared to GFP-injected wild type slices. Additionally, FIGS. 12A and 12B show reduced protein synthesis rates in the FMRP-injected Fmr1 KO slice. These results suggested that FMRP re-expression normalized protein synthesis rates in Fmr1 KO mice, a molecular defect believed to underlie alterations in synaptic plasticity, learning and memory. Overall, the cellular and molecular functional assays performed herein suggested a beneficial effect of low FMRP re-expression in adult Fmr1 KO mice.

### (III) Quantitative Electroencephalograph (EGG)

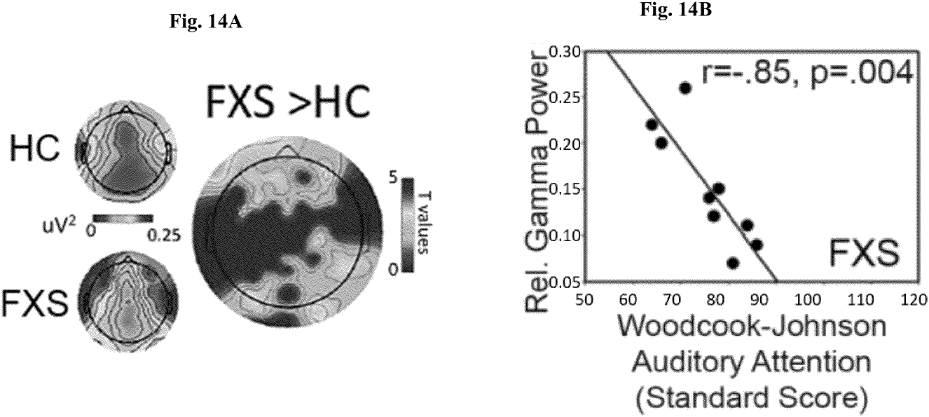

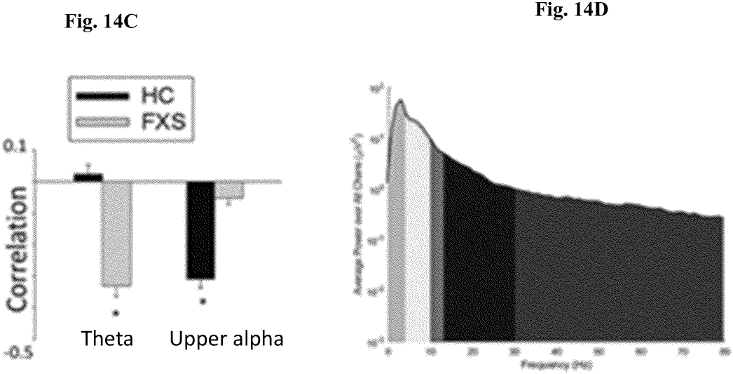

Data provided herein show that quantitative electroencephalography (EEG) can be used as biomarkers of FXS disease severity and treatment response (resting state and auditory event related potentials). FIG. 14A shows a topographical plot of relative gamma power in humans, including significant group differences (p < 0.05 corrected), demonstrating the excessive gamma power observed in FXS patients. Auditory cortex gamma power was highly correlated with behavioral function where higher gamma power was associated with lower performance on auditory attention task in FXS patients (FIG. 14B). The gamma relationships observed with Theta and Alpha power highly discriminate between FXS and healthy human subjects (FIG. 14C). Overall, elevated resting gamma power was found to be a robust quantifiable biomarker of cortical hyperexcitability in humans.

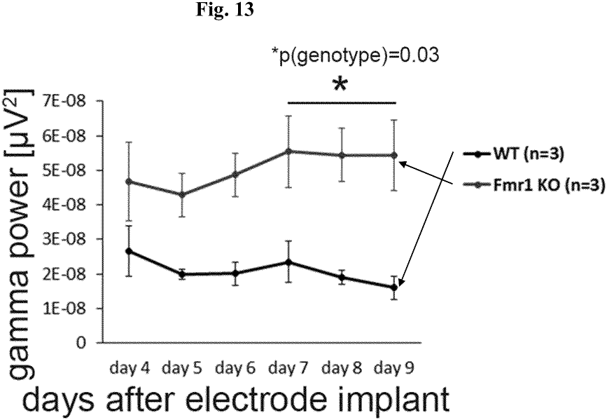

Identification of comparable EEG biomarkers in mouse models of FXS could facilitate the pre-clinical to clinical therapeutic pipeline. To determine if Fmr1 KO mice also display elevated resting gamma power, a 30-channel mouse multielectrode array (MEA) system was used to record and analyze resting and stimulus-evoked EEG signals in wild-type and Fmr1 KO mice. Using this system, robust MEA-derived phenotypes were observed including higher resting EEG power, altered event-related potentials (ERPs) and reduced inter-trial phase coherence to auditory chirp stimuli in Fmr1 KO mice that are remarkably similar to those reported in humans with FXS. FIG. 13 shows increased gamma power in Fmrl KO compared to WT mice where gamma power measured by continuous EEG was calculated for 5-minute periods over 6 days (n=3, RM 2-way ANOVA, *p<0.05). Accordingly, the EEG biomarker of increased resting gamma power found in humans was replicated in Fmr1 KO mice using cortical EEG recordings (FIG. 13).

To correlate changes in mouse EEG biomarkers to human EEG biomarkers, a Matlab based analysis approach was used to parallel mouse data to human data. FIG. 14D shows a gamma power analysis performed and automated using the Matlab-based analysis approach related to abnormalities in FXS using human data. Additional analysis of murine EEG data can assess frequency band-specific EEG power as well as gamma/theta coupling in mice to enable direct comparison of human and murine phenotypes and establish quantitative and translational EEG biomarkers in FXS. Such data may suggest that human EEG biomarkers of FXS could be used as objective measurements in the development and optimization of FXS treatments.

### Example 6. Evaluation of Expression and Biodistribution in FXS Mice in Vivo Following Administration of FMRP-Expressing AAV Vectors

This examples report a pilot preclinical study on FMR1 gene therapy in Fmr1^{KO} or Fmr1^{WT} mice, using AAV-CAGFMR1 (a.k.a., AAV-CB-FMR1) described in Example 1 above or AAV-GFP (as a control). See also FIG. 2A. Male mice, 9.5-11 weeks old, were used in this study. 5×10¹³ vg/kg of the viral particles were injected via tail vein to each mouse. 30 mins to 6 hours later, the mouse was subject to bilateral intracerebroventricular (ICV) surgery and 5×10¹⁰ vg viral particles were delivered to each hemisphere. 12-14 days later, the mouse was sacrificed; blood samples and tissue samples (e.g., brain, muscle, heart, lung, kidney, liver, and spinal cord samples) were collected. Half of the brain samples were analyzed by immunostaining (paraformaldehyde post-fixed). The other half of the brain samples were dissected into hippocampus, cortex, midbrain, and cerebellum (flash-frozen). All brain samples were analyzed by immunostaining for evaluating FMRP expression and distribution. Two sets of other tissue samples (e.g., liver samples) were sectioned, one for detection of GFP expression (imaged directly after cutting without staining to confirm GFP expression), and the other for FMRP expression via immunostaining. The anti-FMRP antibody used in the immunostaining assay is specific to human FMRP with low specific staining in WT mice. Results from this study show neuronal expression of human FMRP, mostly in the cortex.

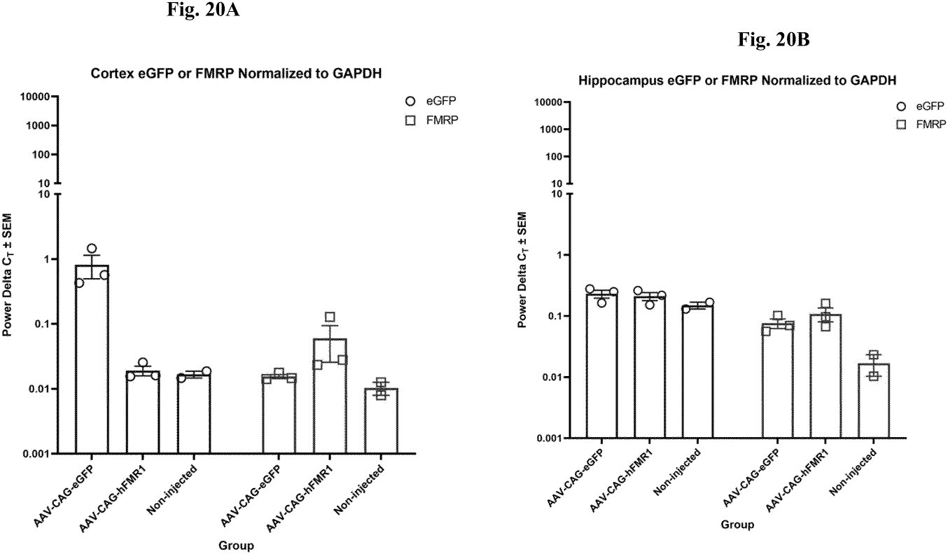

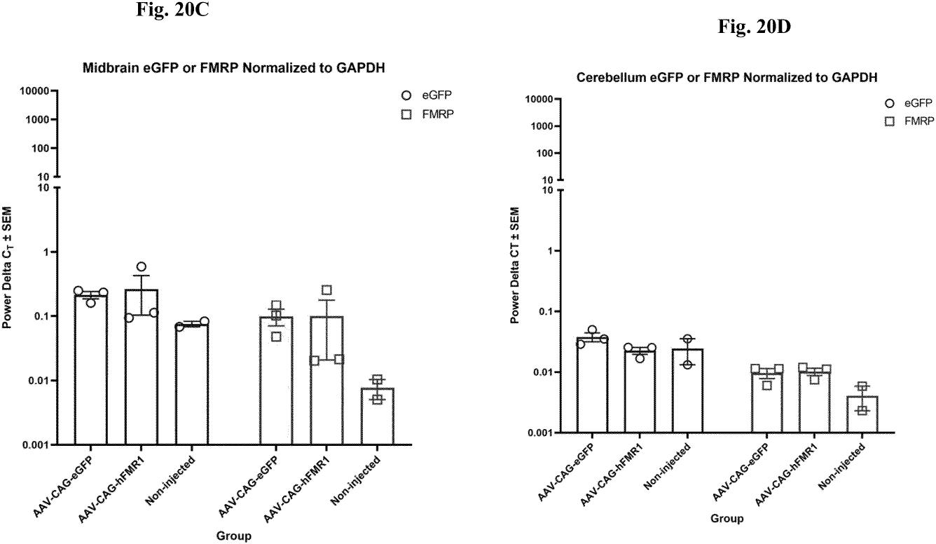

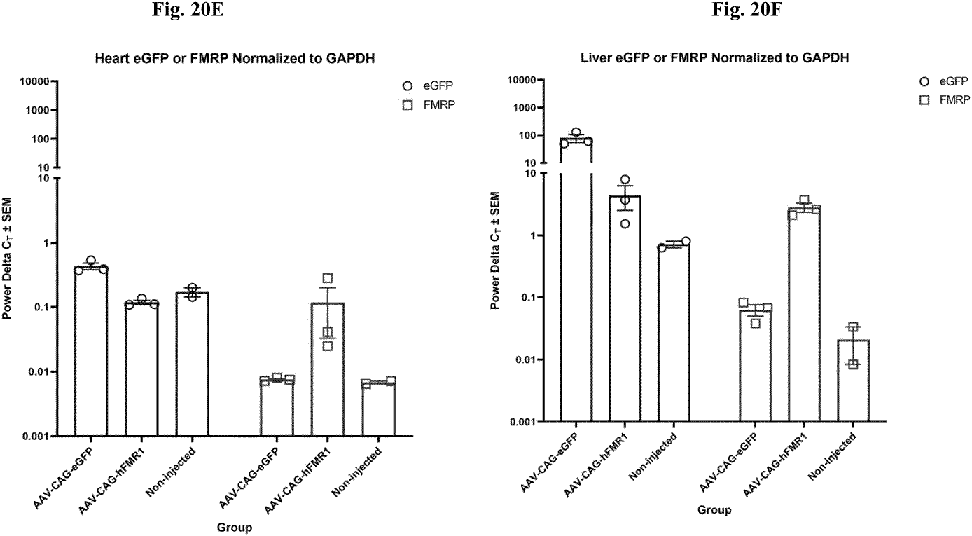

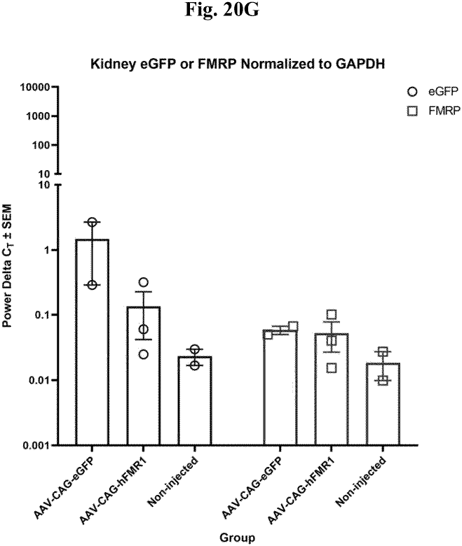

Further, RT-PCR was performed on the brain and tissue samples to detect the level of hFMR1 transcripts in different tissue samples. eGFP was used as a control. The results were normalized to GAPDH and provided in FIGS. 20A-20G. Expression of hFMR1 was detected in various areas in the brain (e.g., cortex) and also in various organs (e.g., heart and liver).

### Example 7. Evaluation of Expression and Biodistribution in FXS Mice in Vivo Following Administration of FMRP-Expressing AAV Vectors

The objective of this study is to further test the distribution and expression of three different viral vectors containing cDNA coding for human FMRP (hPGK, CAGWPRE, and CAGdelWPRE) in Fmr1 knockout (KO) mice. Details of these three vectors are provided in Example 1 above. Viral vector is delivered either intracerebroventricularly (ICV) or intravenously (IV, tail vein) to 5-7 week old mice. After 4 weeks (+/-3 days) blood and organs are harvested and tested for Fmr1 RNA expression by RT-qPCR, and FMRP expression by Western Blot and/or immunohistochemistry (IHC). During the incubation time, mice are monitored for overall health and any adverse reactions.

Brain tissue harvested from mice is analyzed for Fmr1 RNA expression by RT-qPCR, and for FMRP expression by IHC and Western Blot. Other tissues are analyzed for Fmr1 RNA expression by RT-qPCR, and for FMRP expression by Western Blot. Other tissues include dorsal route ganglia (DRG), liver, lung, heart, spinal cord, kidney, gonads, and calf muscle.

The results of the above experiments are analyzed to determine which vector(s) provide superior expression and delivery of transgene to all parts of the brain and body. Given that FMR1 gene is ubiquitously expressed in tissues, it is anticipated that the broad distribution of corrective transgene achievable by one or more of the tested vectors will be beneficial to the treatment of Fragile X Syndrome.

### Example 8. Functional Analyses in FXS Model Mice Following ICV Administration of FMRP-Expressing AAV Vectors: Seizure Susceptibility

The objective of this study is to further assess the effectiveness of three different viral vectors in the reduction of seizure susceptibility in Fragile X Syndrome model mice following treatment with FMRP-expressing AAV vectors administered via ICV administration. FMRP-expressing AAV vectors include CAGWPRE, CAGdelWPRE, and hPGK vectors.

Fmr1 knockout (KO) mice are administered an FMRP-expressing AAV vector via ICV administration at 1-3 days old (P1-3) at a dose of 6e9 vg/ventricle. Control Fmr1 KO mice are administered vehicle at the same age. At age 20-23 days (P20-23), mice undergo Audiogenic Seizure (AGS) testing. P20-P23 mice are placed in a cage with regular bedding without food hopper in groups of two. A personal alarm (120 dB) connected to an A/C power cable is attached to the inside of the cage lid. Sound is played for exactly 2 minutes, followed by 1 minute of silence and another 2 minutes of sound. Mice are observed over the entire duration of the test. Behavior and seizures are scored during both sound exposures. Behavior is scored on a scale of 0-4 as described below:
0 = No change
1 = Wild running
2 = Clonic seizure
3 = Tonic seizure
4 = Death

Mice that survive are put in cages of up to 4, separated by sex. At eight weeks of age, mice that survived AGS testing are euthanized either with CO₂ or pentobarbital. Blood is collected into an EDTA-containing tube through retroorbital bleeding. Mice are then transcardially perfused with sterile PBS. Various organs and tissue are harvested from the mice and subjected to biodistribution analyses. Brain tissue is subjected to RT-qPCR to determine Fmr1 RNA expression, and IHC to probe FMRP expression. Additionally, dorsal root ganglia (DRG), liver, lung, heart, kidney, gonads, and calf muscle tissue are processed and subjected to RT-qPCR to assay Fmr1 RNA expression level.

The results of the above experiments are analyzed to determine which vector(s) provide superior delivery and expression of the transgene, as well as superior rescue of high seizure susceptibility in Fmr1 KO mice. Given that FMR1 gene is ubiquitously expressed in tissues, it is anticipated that the broad distribution of corrective transgene achievable by one or more of the tested vectors will be beneficial to the treatment of Fragile X Syndrome.

### Example 9. Functional Analyses in FXS Model Mice Following ICV, IV, and Combined (IV+ICV) Administration of FMRP-Expressing AAV Vectors

The objective of this study is to further assess rescue of functional neurophysiological deficits in Fragile X Syndrome Model Mice following treatment with FMRP-expressing AAV vectors. FMRP-expressing AAV vectors include CAGWPRE, CAGdelWPRE, and hPGK vectors. The study is performed in two stages (2 cohorts). See Table 3 for Cohort distribution.

TABLE 3

Treatment Groups

| | | | Dosing Cohort (n per group) | |
|---|---|---|---|---|
| Group # | Treatment | Pilot | Cohort 1 | Cohort 2 |
| 1 | KO/neg control (vehicle) | 4 | 6 | 6 |
| 2 | KO/Candidate 1 (pGK) | | 6 | 6 |
| 3 | KO/Candidate 1 (pGK) | | 6 | 6 |
| 4 | KO/Candidate 1 (pGK) | | 6 | 6 |
| 5 | KO/Candidate 2 (CCHMC) | | 6 | 6 |
| 6 | KO/Candidate 3 (CCHMC_delWPRE) | | 6 | 6 |
| 7 | KO/Saline Seizure Check | | 4 | 4 |
| | | | 40 KO, 0 WT | 40 KO, 0 WT |

Mice in groups 1 through 6 receive injections of test AAV vector candidates at 5 weeks of age. Different routes of administration (IV, ICV, and combined IV+ICV) are tested and compared. Mice in all treatment groups are tested for locomotor activity and audiogenic seizure susceptibility (AGS) at 9 weeks of age.

For each cohort, there are seven test groups consisting of 40 mice (see Table 1) which are tested over two consecutive days (AGS testing hours 12:00-4:45). Additionally, the cage changing schedule for each test group is standardized and staggered. Specifically, each test group has their cages changed the day prior to testing.

On test day, mice in groups 1 through 7 are administered saline (IP) 15 min prior to evaluation in the open-field chambers in a locomotor activity (LMA) test. Immediately after the 30 min LMA test, mice are subjected to the AGS test. Mice are then transferred to a clean cage and carried to the AGS testing room individually.

### (I) Locomotor Activity (LMA) Test

Mice are dosed with saline (IP, 10 mL/kg) 15 min prior to being placed in the LMA chambers. Mice are assessed in a 30 minute Open Field Analysis (OFA) using an automated activity monitoring system (MedAssociates). Mice are acclimated to the room 30 min before the start of LMA testing. The following parameters are captured:
Horizontal distance travelled, overall ambulatory time, and ambulatory counts
Vertical activity (time and counts)

### (II) Audiogenic Seizure (AGS) Test

After the LMA procedure, mice are acclimated to the AGS test room for 1 minute. Mice are then placed in a sound-absorptive chamber with a speaker that emits a high intensity tone. Mice are placed (1 at a time) in a clear cylindrical Plexiglas chamber which is placed inside a sound absorptive chamber. The alarm is mounted to the top of the Plexiglas chamber. Behavior of the mice is scored in real-time (see scoring below) by an experimenter who is blinded to genotype status and drug treatments, as well as videotaped for further analysis.

Seizure induction is conducted as follows:

After LMA test, mice are placed into test chamber with attached alarm. After 1 min acclimation, the alarm is started and animal behavior is recorded during a 2 min alarm challenge. The animals are scored based on their behavior. The scoring is as follows:
0 = No response
1 = Wild running
2 = Clonic seizure (lying on side, twitching)
3 = Tonic seizure (lying on side, still)
4 = Respiratory arrest/death.

At t = 3 min. the alarm is turned off and animals are allowed to recover for 1 min. After this recovery, the alarm is restarted and mice are recorded and scored as described above for an additional 2 minutes (from t=4 to t=6 min). After recording, mice are immediately removed from the chamber.

Data are expressed as the magnitude of the seizure event according to the scale described above. Seizure severity score - the average of the highest seizure score for each mouse per group are calculated and analyzed. Also, the percent mice that seize with seizure defined as a seizure score of 2 or more within the 2-minute periods are calculated (seizure incidence).

Immediately following the AGS assay, animals are anesthetized with isoflurane and blood is collected into K2EDTA-coated tubes. Plasma samples are prepared by spinning blood in a refrigerated centrifuge (13,000 rpm and for 3 min at 4° C.). Immediately after blood collection, if applicable, brains are removed and various regions are dissected (e.g., frontal cortex, striatum, hippocampus, cerebellum, brain stem). Plasma is transferred to separate 1.5 mL Eppendorf tubes, frozen, and subjected to bioanalysis. Brains may be flash frozen or immersion fixed in fixative. Animals may also be perfused with saline and fixative prior to brain removal for an additional charge. Optionally, additional organs (e.g., heart, liver, gonads, etc.) may be collected and flash frozen for analysis.

The results of the above experiments are analyzed to determine which administration route provides superior delivery of transgene to all parts of the brain and body, as well as superior rescue of behavioral deficits and high seizure susceptibility in Fmr1 KO mice. Given that FMR1 gene is ubiquitously expressed in tissues, it is anticipated that the broad distribution of corrective transgene achievable by one or more of administration will be beneficial to the treatment of the disease.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ± 20%, preferably up to ± 10%, more preferably up to ± 5%, and more preferably still up to ± 1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating Fragile X Syndrome (FXS), comprising administering to a human patient having FXS an effective amount of a plurality of adeno-associated viral (AAV) 9 viral particles, wherein the AAV9 viral particles comprise a single-stranded AAV DNA vector, which comprises a nucleotide sequence encoding a wild-type human fragile X mental retardation 1 (FMR1) protein (human FMRP), wherein the nucleotide sequence is in operable linkage to a promoter, and wherein the AAV DNA vector expresses the wild-type human FMR1 in the brain of the human patient after infection by the AAV9 viral particles.

2. The method of claim 1, wherein the AAV DNA vector is a self-complementary AAV vector.

3. The method of claim 1, wherein the AAV DNA vector is a standard AAV vector.

4. The method of claim 1, wherein the promoter is a hybrid of a chicken β-actin promoter and a CMV promoter.

5. The method of claim 1, wherein the promoter is a human phosphoglycerate kinase (hPGK) promoter.

6. The method of claim 1, wherein the AAV DNA vector further comprises one or more regulatory elements regulating expression of human FMRP.

7. The method of claim 6, wherein the one or more regulatory elements comprises a human β-globin intron sequence, one or more polyA signaling sequences, a woodchuck hepatitis virus post-transcriptional regulatory element (WPRE), or a combination thereof.

8. The method of claim 7, wherein the one or more polyA signaling sequences comprise a human β-globin polyA signaling sequence, an SV40 polyA signaling sequence, or a combination thereof.

9. The method of claim 4, wherein the AAV DNA vector does not contain a WPRE.

10. The method of claim 1, wherein the AAV DNA vector is a standard AAV vector comprising a hybrid of a chicken β-actin promoter and a CMV promoter in operable linkage to the nucleotide sequence encoding the human FMRP, a WPRE and an SV40 polyA signaling sequence downstream to the nucleotide sequence encoding the human FMRP.

11. The method of claim 1, wherein the AAV DNA vector is a standard AAV vector comprising a hybrid of a chicken β-actin promoter and a CMV promoter in operable linkage to the nucleotide sequence encoding the human FMRP, and an SV40 polyA signaling sequence downstream to the nucleotide sequence encoding the human FMRP, and wherein the AAV DNA vector does not contain a WPRE.

12. The method of claim 1, wherein the AAV DNA vector is a standard AAV vector comprising is a human phosphoglycerate kinase (hPGK) promoter in operable linkage to the nucleotide sequence encoding the human FMRP, a human β-globin intron sequence upstream to the nucleotide sequence encoding the human FMRP, and SV40 polyA signaling and human β-globin polyA signaling sequences downstream to the nucleotide sequence encoding the human FMRP, and wherein the AAV DNA vector does not contain a WPRE.

13. The method of claim 1, wherein the AAV DNA vector further comprises one or more microRNA-target sites (MTSs) specific to one or more tissue-selective microRNAs to suppress expression of the wild-type FMRP in non-brain tissues.

14. The method of claim 13, wherein the one or more MTSs comprise MTS of miR-122, MTS of miR-208a, MTS of miR-208b-3p, MTS of miR-499a-3p, or a combination thereof.

15. The method of claim 1, wherein the wild-type human FMRP is human FMRP isoform 1.

16. The method of claim 1, wherein the human FMRP is a fragment of a wild-type human FMRP comprising the N-terminus 1-297 amino acid residues.

17. The method of claim 1, wherein the AAV9 viral particles are administered to the human patient by intravenous injection, intracerebroventricular injection, intra-cisterna magna injection, intra-parenchymal injection, or a combination thereof.

18. The method of claim 1, wherein the AAV9 viral particles are administered to the human patient via at least two administration routes.

19. The method of claim 18, wherein the at least two administration routes are selected from the group consisting of:
(a) intracerebroventricular injection and intravenous injection;
(b) intrathecal injection and intravenous injection;
(c) intra-cisterna magna injection and intravenous injection; and
(d) intra-parenchymal injection and intravenous injection.

20. The method of claim 1, wherein prior to the administration, the human patient is subject to electroencephalogram (EEG), behavioral and/or cognitive neurorehabilitation assessment, or a combination thereof for determining phenotypic severity of the disease.

21. The method of claim 20, wherein the method further comprises, prior to the administering step, subjecting the human patient to electroencephalogram (EEG), behavioral and/or cognitive neurorehabilitation assessment, or a combination thereof.

22. The method of claim 21, wherein the method further comprises determining dosage of the AAV9 viral particles and/or delivery routes based on the EEG analysis, the behavioral and/or cognitive assessment, or the combination thereof.

23. The method of claim 1, wherein the human patient has been undergoing or is undergoing a treatment comprising a GABA receptor agonist, a PI3K isoform-selective inhibitor, a MMP9 antagonist, or a combination thereof.

24. The method of claim 1, further comprising administering to the human patient an effective amount of a GABA receptor agonist, a PI3K isoform-selective inhibitor, a MMP9 antagonist, or a combination thereof.

25. The method of claim 1, further comprising subjecting the human patient to EEG after administration of the AAV9 viral particles to monitor treatment efficacy.

26. The method of claim 1, further comprising subjecting the human patient to behavioral and/or cognitive neurorehabilitation.

27. The method of claim 26, wherein the neurorehabilitation is performed after administration of the AAV9 viral particles.

28. The method of claim 1, wherein the human patient is a human child.

29. An adeno-associated viral (AAV) vector, comprising:
(i) an AAV backbone, which comprises a 5′ inverted terminal repeats (ITR) and a 3′ ITR;
(ii) a nucleotide sequence encoding a wild-type human fragile X mental retardation 1 (FMR1) protein;
(iii) a promoter in operable linkage to (ii); and
(iv) one or more microRNA-target sites (MTSs) specific to one or more tissue-selective microRNAs to suppress expression of the wild-type FMRP in non-brain tissues.

30. The AAV vector of claim 29, which is a self-complementary AAV vector.

31. The AAV vector of claim 29, wherein the promoter is a hybrid of a chicken β-actin promoter and a CMV promoter.

32. The AAV vector of claim 29, wherein the one or more MTSs comprise MTS of miR-122, MTS of miR-208a, MTS of miR-208b-3p, MTS of miR-499a-3p, or a combination thereof.

33. The AAV vector of claim 29, wherein the wild-type human FMRP is human FMRP isoform 1.

34. A self-complementary adeno-associated viral (AAV) vector, comprising:
(i) an AAV backbone, which comprises a 5′ inverted terminal repeats (ITR) and a truncated 3′ ITR, either one of which or both of which are truncated;
(ii) a nucleotide sequence encoding a wild-type human fragile X mental retardation 1 (FMR1) protein (human FMRP), wherein the wild-type FMRP is FMRP isoform 1; and
(iii) a promoter in operable linkage to (ii).

35. The self-complementary AAV vector of claim 34, further comprising one or more microRNA-target sites (MTSs) specific to one or more tissue-selective microRNAs to suppress expression of the wild-type FMRP in non-brain tissues.

36. The self-complementary AAV vector of claim 34, wherein the promoter is a hybrid of a chicken b-actin promoter and a CMV promoter.

37. The self-complementary AAV vector of claim 34, wherein the one or more MTSs comprise MTS of miR-122, MTS of miR-208a, MTS of miR-208b-3p, MTS of miR-499a-3p, or a combination thereof.

38. A standard adeno-associated viral (AAV) vector, comprising:
(i) an AAV backbone, which comprises a 5′ inverted terminal repeats (ITR) and a 3′ ITR;
(ii) a nucleotide sequence encoding a wild-type human fragile X mental retardation 1 (FMR1) protein;
(iii) a promoter in operable linkage to (ii); and
(iv) one or more regulatory elements regulating expression of the FMRP.

39. The AAV vector of claim 38, wherein the promoter is a hybrid of a chicken β-actin promoter and a CMV promoter or a human phosphoglycerate kinase (hPGK) promoter.

40. The AAV vector of claim 38, wherein the one or more regulatory elements comprises a human β-globin intron sequence, one or more polyA signaling sequences, a woodchuck hepatitis virus post-transcriptional regulatory element (WPRE), or a combination thereof.

41. The AAV vector of claim 40, wherein the one or more polyA signaling sequences comprise a human β-globin polyA signaling sequence, an SV40 polyA signaling sequence, or a combination thereof.

42. The AAV vector of claim 38, wherein the AAV DNA vector does not contain a WPRE.

43. The AAV vector of claim 38, wherein the AAV vector comprises a hybrid of a chicken β-actin promoter and a CMV promoter in operable linkage to the nucleotide sequence encoding the human FMRP, a WPRE and an SV40 polyA signaling sequence downstream to the nucleotide sequence encoding the human FMRP.

44. The AAV vector of claim 38, wherein the AAV vector comprises a hybrid of a chicken β-actin promoter and a CMV promoter in operable linkage to the nucleotide sequence encoding the human FMRP, and an SV40 polyA signaling sequence downstream to the nucleotide sequence encoding the human FMRP, and wherein the AAV DNA vector does not contain a WPRE.

45. The AAV vector of claim 38, wherein the AAV vector comprises a human phosphoglycerate kinase (hPGK) promoter in operable linkage to the nucleotide sequence encoding the human FMRP, a human β-globin intron sequence upstream to the nucleotide sequence encoding the human FMRP, and SV40 polyA signaling and human β-globin polyA signaling sequences downstream to the nucleotide sequence encoding the human FMRP, and wherein the AAV DNA vector does not contain a WPRE.

46. An adeno-associated viral (AAV) 9 viral particle, comprising an AAV9 capsid encapsulating a single-stranded AAV DNA vector, wherein the AAV DNA vector is set forth in claim 29.

47. A pharmaceutical composition, comprising the AAV9 viral particle of claim 46 and a pharmaceutically acceptable carrier.


SEQUENCE LISTING (6 sequences)

SEQ ID NO: 1 - protein, 632 aa, Artificial Sequence [sequence omitted]

SEQ ID NO: 2 - DNA, 899 bp, Artificial Sequence
  Synthetic
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 3 - DNA, 511 bp, Artificial Sequence
  Synthetic
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 4 - DNA, 9239 bp, Artificial Sequence
  Synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatgtcgacaccggtggccggcccagccctgactgctatggacttttagtgcttgtgcactgctaccagccatgccctgagtgcacagggttaccctggctggggcctggaagcatataaaagaagctcagagtcacatccaggtacacctttcacctgggccagcttcattcagaaggccaggagggggggattttgccatgtctgaagttccagttataggcacacagttgcatcttactaggtctggtctagggatcaccagtgacttccagcctctaattccacactgttcagcaggcacctgttgtggcaggctggtcttagttagctagggcagggtttcttcactgggagtcctggcattcagaatgattaagggctccctctgcctacctatccccagtcaaccagccccaggtgtgtagctctagtctgagactcatatatccagggtcacctcctgggatcaagtgcatgagcccaagctgggatctttccccagttacatatttctggacagtagggctcagagtctccagtgccagtcctgtttcagagtatggaaaaagtaaccattgttacagccacagtccttgggttagccctaactcagctaatccagcaggcccaaggacacctgaatatccaaggtatgagtgtgaggccctgtaaggtaattctagccccttatgcctaacttgattatcaagaccaagctatatcagggatgaaccagaggctccatgacaccccagccacctagctaaacttgggggttgggtacaagttagcccagaacataccttatagaattgcctctctagggtgagcaagggccaacctgcctatttgccctctcacccccattgcaatagctttggctcccagtacctcttccctggcttcattagcagatggcacccagcagataaaggtctatacccctgacaagggaaacatggaaagtatcaggaccaacatggtccacagcagaagtgtctggagtccatcctgcatggccttgagtccaggcacaggagtctccagtgagggtaaaccccaatcattgtccatccaggttttgccataagacttgggccagggtagctaaagcagatttacccctgcaaggagacacctcattggaaactgaaagagactccccaccagcttgaaaggccagtcatgcttttgcctgactcctgctctctatgcagtggcaatctaagtgggaggtctgttcttcccaagagaggaccaagtttctgtcccaaggcaataatcctgttatcattggctcctagctgccattgttctgattgagggtttaaactccggaatttaaatcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcaggcctcctaggcttgcatgcagtactattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccccgggtaccggtgccaccatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctgataagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttagatctgtgtgttggttttttcttaaggtgtgattaatgagctaccaggtctcgaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggttataacctgcaggttaattaagccttgtagtctagccagggtgtataacccctccagcccatgttcaaagagctgtcttgcctagccagttgcacatacaaagtgataaatgggagctggcattgtgcccttggaggcactctgaaggatcaccagtgaacccagcagcaaagatacataggctcttaattcatgccaggatctcagagaggcaatggcttgcacaatcaggggttattctgacagcatgaactctggtgccagacaattttatgtatcaggcaatgtgcattacttgaggtggattacagacccagtgagtaacccaggaccaggagtaaacaggccctaatcccagcttgattgacaccaggcttgaagatccttaccattatgaaaacagcacatggtctgttgacaattacttgtagcataggtagcccaggcagagtggcagacagggtactgatagtagttagagaacctcccagataagctacctgaccttctctaatcttgagttctgtgggcaaccagcccagtgagcatcttgggttcttggaaatccagaccctactcacctgagtataaatggggcagccacccacccagactgatccatcctacctttgaggctacccatgaggtaattaggccagaatgtgaaagggaggagcccagagcattgttccttgtattaccattgggaaccttgttagatggggaggaatgcacttcagccaccctagaggagttgagtcattagaagaagaaggctgcttccccatcaggagaagatccagccagtaagtatataggttacaactgccaggtactatgggcttctccagacccttcctacccaggaactagaaggttggagcctaaagtcccctacccatgtgctgactgatccagagttacactccctcagactcatcctccagacagggttcccagttattaggattgcaataaccatccagttcccaaactattccagcttcctatccagtaataagcccttattctttaacctctgaagaaaccctgagtgaacaggctgtgcagggctcctgtcacttctgtcagcccagataggtatgaaatctcttcaaatgtattgccaatgacattgccaggcacagattctcccagttacccaaccaggaacaccagctagtgtcaaacagtcaatgcccttctaccagccatttgagacactaccaggcaggcaaagccaagtggcttcactccttatttatattagctcagaggagccatctaacagctcttactcaactagactagctgcatgaacagtgtacagctagctgcgcaatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 5 - DNA, 8650 bp, Artificial Sequence
  Synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatgtcgacaccggtggccggcccagccctgactgctatggacttttagtgcttgtgcactgctaccagccatgccctgagtgcacagggttaccctggctggggcctggaagcatataaaagaagctcagagtcacatccaggtacacctttcacctgggccagcttcattcagaaggccaggagggggggattttgccatgtctgaagttccagttataggcacacagttgcatcttactaggtctggtctagggatcaccagtgacttccagcctctaattccacactgttcagcaggcacctgttgtggcaggctggtcttagttagctagggcagggtttcttcactgggagtcctggcattcagaatgattaagggctccctctgcctacctatccccagtcaaccagccccaggtgtgtagctctagtctgagactcatatatccagggtcacctcctgggatcaagtgcatgagcccaagctgggatctttccccagttacatatttctggacagtagggctcagagtctccagtgccagtcctgtttcagagtatggaaaaagtaaccattgttacagccacagtccttgggttagccctaactcagctaatccagcaggcccaaggacacctgaatatccaaggtatgagtgtgaggccctgtaaggtaattctagccccttatgcctaacttgattatcaagaccaagctatatcagggatgaaccagaggctccatgacaccccagccacctagctaaacttgggggttgggtacaagttagcccagaacataccttatagaattgcctctctagggtgagcaagggccaacctgcctatttgccctctcacccccattgcaatagctttggctcccagtacctcttccctggcttcattagcagatggcacccagcagataaaggtctatacccctgacaagggaaacatggaaagtatcaggaccaacatggtccacagcagaagtgtctggagtccatcctgcatggccttgagtccaggcacaggagtctccagtgagggtaaaccccaatcattgtccatccaggttttgccataagacttgggccagggtagctaaagcagatttacccctgcaaggagacacctcattggaaactgaaagagactccccaccagcttgaaaggccagtcatgcttttgcctgactcctgctctctatgcagtggcaatctaagtgggaggtctgttcttcccaagagaggaccaagtttctgtcccaaggcaataatcctgttatcattggctcctagctgccattgttctgattgagggtttaaactccggaatttaaatcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcaggcctcctaggcttgcatgcagtactattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccccgggtaccggtgccaccatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctgataagaattcgatatcaagcttatcgatatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttagatctgtgtgttggttttttcttaaggtgtgattaatgagctaccaggtctcgaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggttataacctgcaggttaattaagccttgtagtctagccagggtgtataacccctccagcccatgttcaaagagctgtcttgcctagccagttgcacatacaaagtgataaatgggagctggcattgtgcccttggaggcactctgaaggatcaccagtgaacccagcagcaaagatacataggctcttaattcatgccaggatctcagagaggcaatggcttgcacaatcaggggttattctgacagcatgaactctggtgccagacaattttatgtatcaggcaatgtgcattacttgaggtggattacagacccagtgagtaacccaggaccaggagtaaacaggccctaatcccagcttgattgacaccaggcttgaagatccttaccattatgaaaacagcacatggtctgttgacaattacttgtagcataggtagcccaggcagagtggcagacagggtactgatagtagttagagaacctcccagataagctacctgaccttctctaatcttgagttctgtgggcaaccagcccagtgagcatcttgggttcttggaaatccagaccctactcacctgagtataaatggggcagccacccacccagactgatccatcctacctttgaggctacccatgaggtaattaggccagaatgtgaaagggaggagcccagagcattgttccttgtattaccattgggaaccttgttagatggggaggaatgcacttcagccaccctagaggagttgagtcattagaagaagaaggctgcttccccatcaggagaagatccagccagtaagtatataggttacaactgccaggtactatgggcttctccagacccttcctacccaggaactagaaggttggagcctaaagtcccctacccatgtgctgactgatccagagttacactccctcagactcatcctccagacagggttcccagttattaggattgcaataaccatccagttcccaaactattccagcttcctatccagtaataagcccttattctttaacctctgaagaaaccctgagtgaacaggctgtgcagggctcctgtcacttctgtcagcccagataggtatgaaatctcttcaaatgtattgccaatgacattgccaggcacagattctcccagttacccaaccaggaacaccagctagtgtcaaacagtcaatgcccttctaccagccatttgagacactaccaggcaggcaaagccaagtggcttcactccttatttatattagctcagaggagccatctaacagctcttactcaactagactagctgcatgaacagtgtacagctagctgcgcaatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 6 - DNA, 9277 bp, Artificial Sequence
  Synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatgtcgacaccggtggccggcccagccctgactgctatggacttttagtgcttgtgcactgctaccagccatgccctgagtgcacagggttaccctggctggggcctggaagcatataaaagaagctcagagtcacatccaggtacacctttcacctgggccagcttcattcagaaggccaggagggggggattttgccatgtctgaagttccagttataggcacacagttgcatcttactaggtctggtctagggatcaccagtgacttccagcctctaattccacactgttcagcaggcacctgttgtggcaggctggtcttagttagctagggcagggtttcttcactgggagtcctggcattcagaatgattaagggctccctctgcctacctatccccagtcaaccagccccaggtgtgtagctctagtctgagactcatatatccagggtcacctcctgggatcaagtgcatgagcccaagctgggatctttccccagttacatatttctggacagtagggctcagagtctccagtgccagtcctgtttcagagtatggaaaaagtaaccattgttacagccacagtccttgggttagccctaactcagctaatccagcaggcccaaggacacctgaatatccaaggtatgagtgtgaggccctgtaaggtaattctagccccttatgcctaacttgattatcaagaccaagctatatcagggatgaaccagaggctccatgacaccccagccacctagctaaacttgggggttgggtacaagttagcccagaacataccttatagaattgcctctctagggtgagcaagggccaacctgcctatttgccctctcacccccattgcaatagctttggctcccagtacctcttccctggcttcattagcagatggcacccagcagataaaggtctatacccctgacaagggaaacatggaaagtatcaggaccaacatggtccacagcagaagtgtctggagtccatcctgcatggccttgagtccaggcacaggagtctccagtgagggtaaaccccaatcattgtccatccaggttttgccataagacttgggccagggtagctaaagcagatttacccctgcaaggagacacctcattggaaactgaaagagactccccaccagcttgaaaggccagtcatgcttttgcctgactcctgctctctatgcagtggcaatctaagtgggaggtctgttcttcccaagagaggaccaagtttctgtcccaaggcaataatcctgttatcattggctcctagctgccattgttctgattgagggtttaaactccggaatttaaatcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcaggcctcctaggcttgcatgcagtactggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggaattcaggtacctgagctctgaggatccttatcagccaccatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaatgacacattgtgtgatatctctaggatgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttaagatctgtgttaagatacattgatgagtttggacaaaccacaactagaatgcagtgaaaaaaatgctttatttgtgaaatttgtgatgctattgctttatttgtaaccattataagctgcaataaacaagttatgcatggacatgtttgatcatggttgtaagtatcaaggttacaagacaggtttaaggagaccaatagaaactgggcttgtcgagacagagaagactcttgcgtttctgataggcacctattggtcttactgacatccactttgcctttctctccacagagatctgtgtgttggttttttcttaaggtgtgattaatgagctaccaggtctcgaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggttataacctgcaggttaattaagccttgtagtctagccagggtgtataacccctccagcccatgttcaaagagctgtcttgcctagccagttgcacatacaaagtgataaatgggagctggcattgtgcccttggaggcactctgaaggatcaccagtgaacccagcagcaaagatacataggctcttaattcatgccaggatctcagagaggcaatggcttgcacaatcaggggttattctgacagcatgaactctggtgccagacaattttatgtatcaggcaatgtgcattacttgaggtggattacagacccagtgagtaacccaggaccaggagtaaacaggccctaatcccagcttgattgacaccaggcttgaagatccttaccattatgaaaacagcacatggtctgttgacaattacttgtagcataggtagcccaggcagagtggcagacagggtactgatagtagttagagaacctcccagataagctacctgaccttctctaatcttgagttctgtgggcaaccagcccagtgagcatcttgggttcttggaaatccagaccctactcacctgagtataaatggggcagccacccacccagactgatccatcctacctttgaggctacccatgaggtaattaggccagaatgtgaaagggaggagcccagagcattgttccttgtattaccattgggaaccttgttagatggggaggaatgcacttcagccaccctagaggagttgagtcattagaagaagaaggctgcttccccatcaggagaagatccagccagtaagtatataggttacaactgccaggtactatgggcttctccagacccttcctacccaggaactagaaggttggagcctaaagtcccctacccatgtgctgactgatccagagttacactccctcagactcatcctccagacagggttcccagttattaggattgcaataaccatccagttcccaaactattccagcttcctatccagtaataagcccttattctttaacctctgaagaaaccctgagtgaacaggctgtgcagggctcctgtcacttctgtcagcccagataggtatgaaatctcttcaaatgtattgccaatgacattgccaggcacagattctcccagttacccaaccaggaacaccagctagtgtcaaacagtcaatgcccttctaccagccatttgagacactaccaggcaggcaaagccaagtggcttcactccttatttatattagctcagaggagccatctaacagctcttactcaactagactagctgcatgaacagtgtacagctagctgcgcaatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca